(19) United States
(12) Patent Application Publication
(10) Pub. No.: US 2016/0211456 A1
(43) Pub. Date: Jul. 21, 2016

(54) PHENANTHRO[9,10-b]TETRAPHENYLENE DERIVATIVE AND USE THEREOF

(71) Applicant: LUMINESCENE TECHNOLOGY CORP., Hsin-Chu (TW)

(72) Inventors: Feng-Wen Yen, Taipei (TW); Cheng-Hao Chang, Miao-Li (TW)

(73) Assignee: Luminescence Technology Corp.

(21) Appl. No.: 14/599,566

(22) Filed: Jan. 19, 2015

(51) Int. Cl. H01L 51/00; C09K 11/06; C07C 211/61; C07D 471/04; C07D 251/24; C07D 239/26; C07D 219/02; C07D 209/86; C07D 403/10; C07D 279/22; C07D 333/76; C07D 265/38; C07D 403/14; C07D 403/04; H01L 51/50; C07D 307/91

(52) U.S. Cl. CPC H01L 51/0056; C07D 307/91; C09K 11/06; H01L 51/0073; C07C 211/61; H01L 51/006; C07D 471/04; H01L 51/0072; C07D 251/24; H01L 51/0067; C07D 239/26; C07D 219/02; C07D 209/86; C07D 403/10; C07D 279/22; H01L 51/0071; C07D 333/76; H01L 51/0074; C07D 265/38; C07D 403/14; C07D 403/04; H01L 51/5016; C09K 2211/1018; C09K 2211/1014; C09K 2211/1011; C09K 2211/1007; H01L 51/5206

(57) ABSTRACT

The present invention discloses a novel phenanthro[9,10-b]tetraphenylene derivative is represented by the following formula(I), the organic EL device employing the phenanthro[9,10-b]tetraphenylene derivative as electron blocking material, hole blocking material, electron transport material, phosphorescent host material, or fluorescent host and dopant material can display good performance.

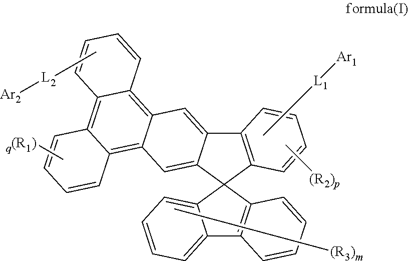

wherein L₁, L₂, Ar₁, Ar₂, m, p, q and R₁ to R₃ are the same definition as described in the present invention.

### FIELD OF INVENTION

The present invention generally relates to a novel phenanthro [9,10-b]tetraphenylene derivative and organic electroluminescent (herein referred to as organic EL) device using the phenanthro[9, 10-b]tetraphenylene derivative. More specifically, the present invention relates to the phenanthro [9,10-b]tetraphenylene derivative having general formula(I), an organic EL device employing the phenanthro[9, 10-b]tetraphenylene derivative as emitting host or dopant, hole blocking layer(HBL), electron blocking layer(EBL), electron transport layer(ETL) and hole transport layer(HTL).

### BACKGROUND OF THE INVENTION

Organic electroluminescent(organic EL) is a light-emitting diode (LED) in which the emissive layer is a film made by organic compounds which emits light in response to an electric current. The emissive layer of organic compound is sandwiched between two electrodes. Organic EL is applied in flat panel displays due to their high illumination, low weight, ultra-thin profile, self-illumination without back light, low power consumption, wide viewing angle, high contrast, simple fabrication methods and rapid response time.

The first observation of electroluminescence in organic materials were in the early 1950s by Andre Bernanose and co-workers at the Nancy-University in France. Martin Pope and his co-workers at New York University first observed direct current(DC) electroluminescence on a single pure crystal of anthracene and on anthracene crystals doped with tetracene under vacuum in 1963.

The first diode device was reported by Ching W. Tang and Steven Van Slyke at Eastman Kodak in 1987. The device used a two-layer structure with separate hole transporting and electron transporting layers resulted in reduction in operating voltage and improvement of the efficiency, that led to the current era of organic EL research and device production.

Typically organic EL device is composed of layers of organic materials situated between two electrodes, which include a hole transporting layer(HTL), an emitting layer(EML), an electron transporting layer(ETL). The basic mechanism of organic EL involves the injection of the carrier, transport, recombination of carriers and exciton formed to emit light. When an external voltage is applied to an organic EL device, electrons and holes are injected from a cathode and an anode, respectively, electrons will be injected from a cathode into a LUMO(lowest unoccupied molecular orbital) and holes will be injected from an anode into a HOMO(highest occupied molecular orbital). When the electrons recombine with holes in the emitting layer, excitons are formed and then emit light. When luminescent molecules absorb energy to achieve an excited state, an exciton may either be in a singlet state or a triplet state depending on how the spins of the electron and hole have been combined. 75% of the excitons form by recombination of electrons and holes to achieve a triplet excited state. Decay from triplet states is spin forbidden, Thus, a fluorescence electroluminescent device has only 25% internal quantum efficiency. In contrast to fluorescence electroluminescent device, phosphorescent organic EL device make use of spin-orbit interactions to facilitate intersystem crossing between singlet and triplet states, thus obtaining emission from both singlet and triplet states and the internal quantum efficiency of electroluminescent devices from 25% to 100%.

Recently, a new type of fluorescent organic EL device incorporating mechanism of thermally activated delayed fluorescence(TADF) has been developed by Adachi and coworkers is a promising way to obtain a high efficiency of exciton formation by converting spin-forbidden triplet excitons up to the siglet level by the mechanism of reverse intersystem crossing (RISC).

The phosphorescent organic EL utilizes both triplet and singlet excitons. Cause of longer lifetime and the diffusion length of triplet excitons compared to those of singlet excitons, the phosphorescent organic EL generally need an additional hole-blocking layer(HBL) between the emitting layer(EML) and the electron transporting layer(ETL) or the electron transporting layer with hole blocking ability instead of typical ETL. The purpose of the use of HBL or HBETL is to confine the recombination of injected holes and electrons and the relaxation of created excitons within the EML, hence the device's efficiency can be improved. To meet such roles, the hole blocking materials must have HOMO(highest occupied molecular orbital) and LUMO(lowest unoccupied molecular orbital) energy levels suitable to block hole transport from the EML to the ETL and to pass electrons from the ETL to the EML, in addition, the good thermal and electrochemical stability of the materials are also needed.

There continues to be a need for organic EL materials which is able to efficiently transport electrons or holes and block holes, with good thermal stability and more efficient EML materials for high emitting efficiency. According to the reasons described above, the present invention has the objective of resolving such problems of the prior-art and offering a light emitting device which is excellent in its thermal stability, high luminance efficiency, high luminance and long half-life time. The present invention disclose a novel phenanthro[9,10-b]tetraphenylene derivative having general formula(I), used as emitting host or dopant, hole blocking layer(HBL), electron blocking layer(EBL), electron transport layer(ETL) and hole transport layer(HTL) have good charge carrier mobility and excellent operational durability can lower driving voltage and power consumption, increasing efficiency and half-life time of organic EL device.

### SUMMARY OF THE INVENTION

A novel phenanthro[9,10-b]tetraphenylene derivative can use as emitting host or dopant, hole blocking layer(HBL), electron blocking layer(EBL), electron transport layer(ETL) and hole transport layer(HTL) for organic EL and their use for organic EL device are provided. The phenanthro[9,10-b]tetraphenylene derivative can overcome the drawbacks of the conventional materials like as shorter half-life time, lower efficiency and higher power consumption.

An object of the present invention is to provide the phenanthro[9,10-b]tetraphenylene derivative which can be used as hole blocking layer(HBL) material, electron blocking layer(EBL) material for organic EL device and can efficiently confine excitons to transfer to electron transport layer or hole transport layer.

An object of the present invention is to provide the phenanthro[9,10-b]tetraphenylene derivative which can be used as phosphorescent host material, fluorescenct host material or fluorescenct dopant of emitting layer for organic EL device and increase the efficiency.

Another object of the present invention is to provide the phenanthro[9,10-b]tetraphenylene derivative which can be used as hole transport layer(HTL) material, electron transport layer(ETL) material for organic EL device and improve the half-life time, lower driving voltage and lower power consumption.

The present invention has the economic advantages for industrial practice. Accordingly the present invention, the phenanthro[9, 10-b]tetraphenylene derivative which can be used for organic EL device is disclosed. The mentioned the phenanthro[9,10-b]tetraphenylene derivative is represented by the following formula(I):

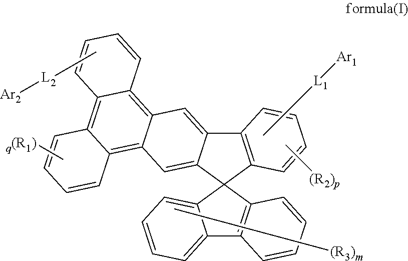

Wherein L₁, L₂ represent a single bond, a substituted or unsubstituted arylene group having 6 to 30 ring carbon atoms, or a substituted or unsubstituted heterarylene group having 3 to 30 ring carbon atoms. m represent an integer of 0 to 8. p represent an integer of 0 to 3, q represent an integer of 0 to 9. R₁ to R₃ independently selected from the group consisting of a hydrogen atom, a halide, alkyl group having 1 to 20 carbon atoms, a substituted or unsubstituted aryl group having 6 to 30 carbon atoms, a substituted or unsubstituted aralkyl group having 6 to 30 carbon atoms, a substituted or unsubstituted heteroaryl group having 3 to 30 carbon atoms. Ar₁ and Ar₂ independently represent a substituted or unsubstituted arylamine, a substituted or unsubstituted heteroarylamine, a substituted or unsubstituted aryl group having 6 to 50 carbon atoms, a substituted or unsubstituted heteroaryl group having 3 to 50 carbon atoms. wherein at least one of Ar₁ and Ar₂ represent a substituted or unsubstituted diphenylamine group, a substituted or unsubstituted N-phenylnaphthalene-2-amine group, a substituted or unsubstituted dibiphenyl-4-ylamine group, a substituted or unsubstituted N-phenyldibenzo[b,d]furan-4-amine group, a substituted or unsubstituted phenyl group, a substituted or unsubstituted naphthyl group, a substituted or unsubstituted anthracenyl group, a substituted or unsubstituted phenanthrenyl group, a substituted or unsubstituted pyrenyl group, a substituted or unsubstituted chrysenyl group, a substituted or unsubstituted triphenylenyl group, a substituted or unsubstituted perylenyl group, a substituted or unsubstituted carbazolyl group, a substituted or unsubstituted biscarbazolyl group, a substituted or unsubstituted dibenzofuranyl group, a substituted or unsubstituted dibenzothiophenyl group, a substituted or unsubstituted triazinyl group, a substituted or unsubstituted diazinyl group, a substituted or unsubstituted phenanthroline group, a substituted or unsubstituted dihydroacridine group, a substituted or unsubstituted phenothiazine group, a substituted or unsubstituted phenoxazine group, a substituted or unsubstituted dihydrophenazine group and the substituent for Ar₁, Ar₂ each have the same definition as R₁.

### BRIEF DESCRIPTION OF THE DRAWINGS

FIG. 1 show one example of organic EL device in the present invention. 6 is transparent electrode, 13 is metal electrode, 7 is hole injection layer which is deposited onto 6, 8 is hole transport layer which is deposited onto 7, 9 is fluorescent or phosphorescent emitting layer which is deposited onto 8, 10 is hole blocking layer which is deposited onto 9, 11 is electron transport layer which is deposited onto 10, 12 is electron injection layer which is deposited on to 11.

FIG. 2 show one example of organic EL device in the present invention. 6 is transparent electrode, 13 is metal electrode, 7 is hole injection layer which is deposited onto 6, 8 is hole transport layer which is deposited onto 7, 9 is electron blocking layer which is deposited onto 8, 10 is fluorescent or phosphorescent emitting layer which is deposited onto 9, 11 is electron transport layer which is deposited onto 10, 12 is electron injection layer which is deposited on to 11.

FIG. 3 show one example of organic EL device in the present invention. 6 is transparent electrode, 14 is metal electrode, 7 is hole injection layer which is deposited onto 6, 8 is hole transport layer which is deposited onto 7, 9 is electron blocking layer which is deposited onto 8, 10 is fluorescent or phosphorescent emitting layer which is deposited onto 9, 11 is hole blocking layer which is deposited onto 10, 12 is electron transport layer which is deposited onto 11, 13 is electron injection layer which is deposited on to 12.

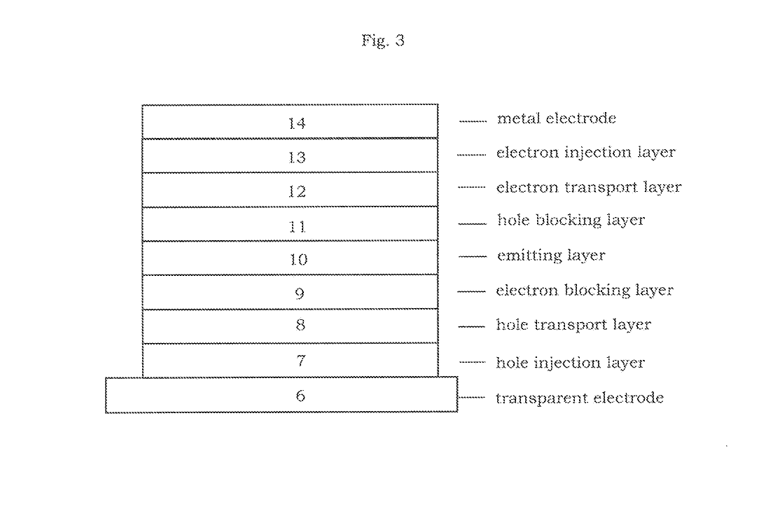

FIG. 4 show the ¹HNMR of 12-bromophenanthro[9,10-b]tetraphenylene which is important synthetic intermediate of phenanthro [9,10-b]tetraphenylene skeleton for the present invention formula(I).

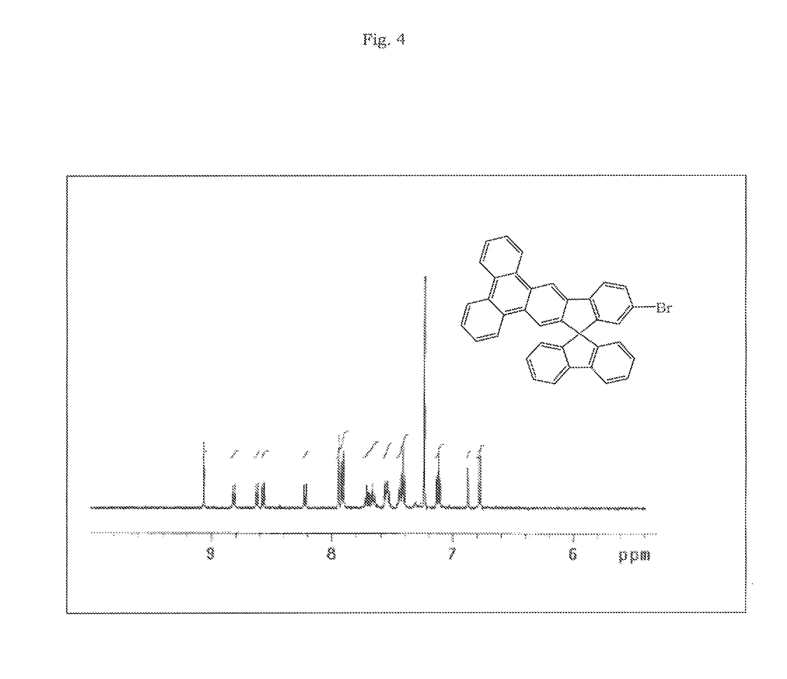

FIG. 5 show the ¹HNMR of 3-methoxyphenanthro[9,10-b]tetra phenylene which is important synthetic intermediate of phenanthro [9, 10-b]tetraphenylene skeleton for the present invention formula(I).

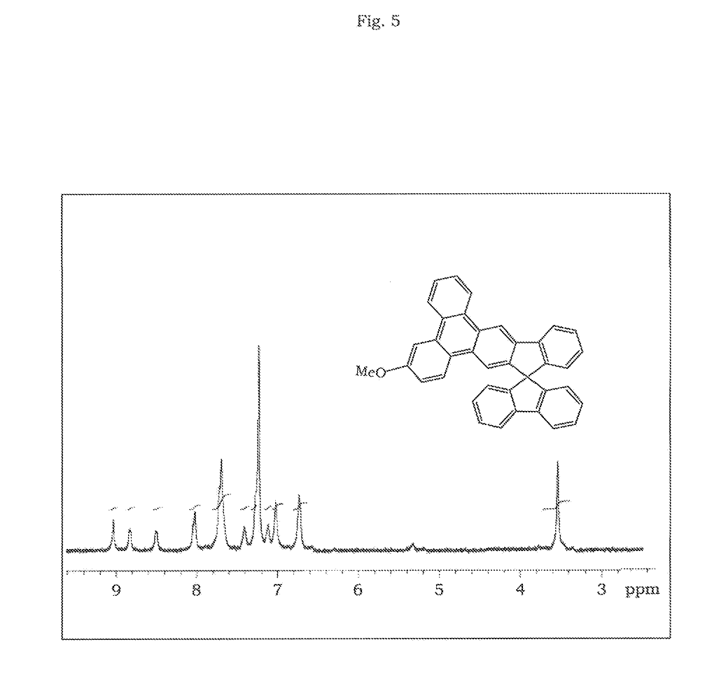

### DESCRIPTION OF THE PREFERRED EMBODIMENTS

What probed into the invention is the phenanthro[9,10-b]tetraphenylene derivative and organic EL device using the phenanthro[9,10-b]tetraphenylene derivative. Detailed descriptions of the production, structure and elements will be provided in the following to make the invention thoroughly understood. Obviously, the application of the invention is not confined to specific details familiar to those who are skilled in the art. On the other hand, the common elements and procedures that are known to everyone are not described in details to avoid unnecessary limits of the invention. Some preferred embodiments of the present invention will now be described in greater detail in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

In a first embodiment of the present invention, the phenanthro[9,10-b]tetraphenylene derivative which can be used as emitting host or dopant, hole blocking layer(HBL), electron blocking layer(EBL), electron transport layer(ETL) and hole transport layer(HTL) for organic EL device are disclosed. The mentioned material are represented by the following formula(I):

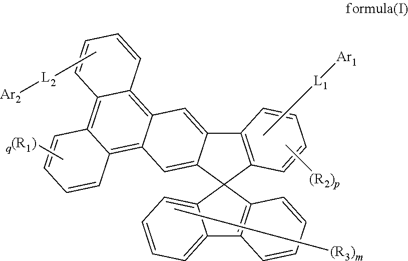

Wherein L₁, L₂ represent a single bond, a substituted or unsubstituted arylene group having 6 to 30 ring carbon atoms, or a substituted or unsubstituted heterarylene group having 3 to 30 ring carbon atoms. m represent an integer of 0 to 8. p represent an integer of 0 to 3, q represent an integer of 0 to 9. R₁ to R₃ independently selected from the group consisting of a hydrogen atom, a halide, alkyl group having 1 to 20 carbon atoms, a substituted or unsubstituted aryl group having 6 to 30 carbon atoms, a substituted or unsubstituted aralkyl group having 6 to 30 carbon atoms, a substituted or unsubstituted heteroaryl group having 3 to 30 carbon atoms. Ar₁ and Ar₂ independently represent a substituted or unsubstituted arylamine, a substituted or unsubstituted heteroarylamine, a substituted or unsubstituted aryl group having 6 to 50 carbon atoms, a substituted or unsubstituted heteroaryl group having 3 to 50 carbon atoms. wherein at least one of Ar₁ and Ar₂ represent a substituted or unsubstituted diphenylamine group, a substituted or unsubstituted N-phenylnaphthalene-2-amine group, a substituted or unsubstituted dibiphenyl-4-ylamine group, a substituted or unsubstituted N-phenyldibenzo[b,d]furan-4-amine group, a substituted or unsubstituted phenyl group, a substituted or unsubstituted naphthyl group, a substituted or unsubstituted anthracenyl group, a substituted or unsubstituted phenanthrenyl group, a substituted or unsubstituted pyrenyl group, a substituted or unsubstituted chrysenyl group, a substituted or unsubstituted triphenylenyl group, a substituted or unsubstituted perylenyl group, a substituted or unsubstituted carbazolyl group, a substituted or unsubstituted biscarbazolyl group, a substituted or unsubstituted dibenzofuranyl group, a substituted or unsubstituted dibenzothiophenyl group, a substituted or unsubstituted triazinyl group, a substituted or unsubstituted diazinyl group, a substituted or unsubstituted phenanthroline group, a substituted or unsubstituted dihydroacridine group, a substituted or unsubstituted phenothiazine group, a substituted or unsubstituted phenoxazine group, a substituted or unsubstituted dihydrophenazine group and the substituent for Ar₁, Ar₂ each have the same definition as R₁.

Some preferably examples for Ar₁ and Ar₂ are consisting of group represent as following:

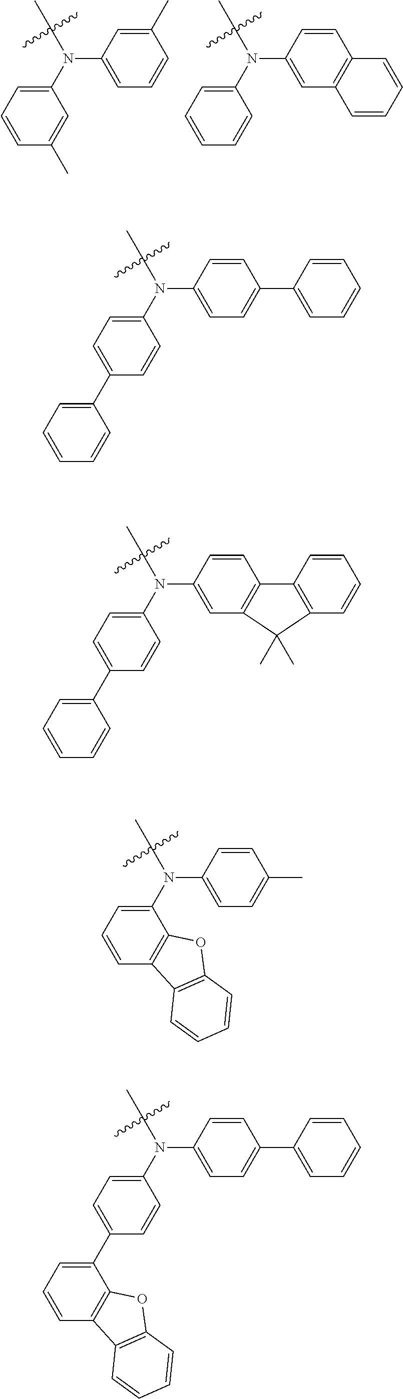

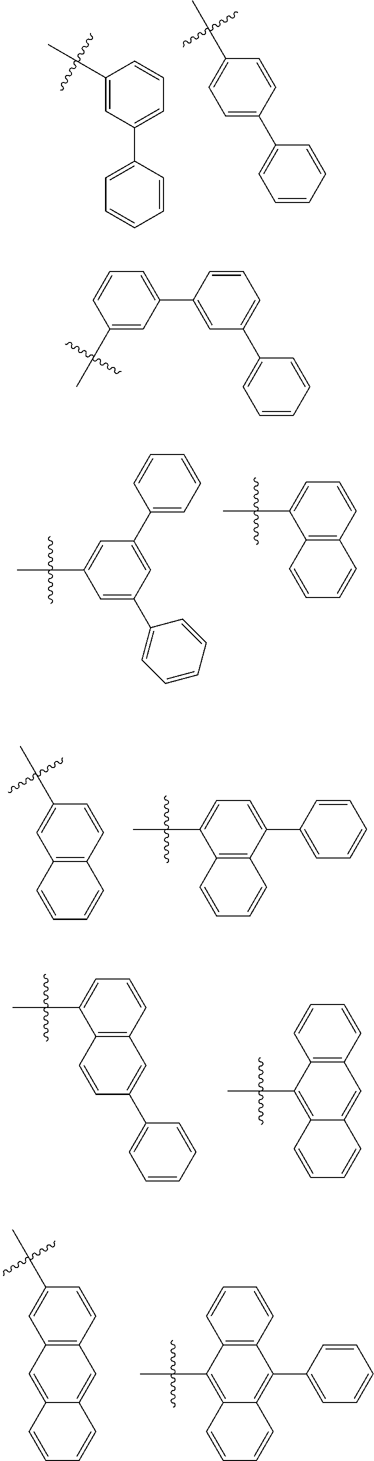

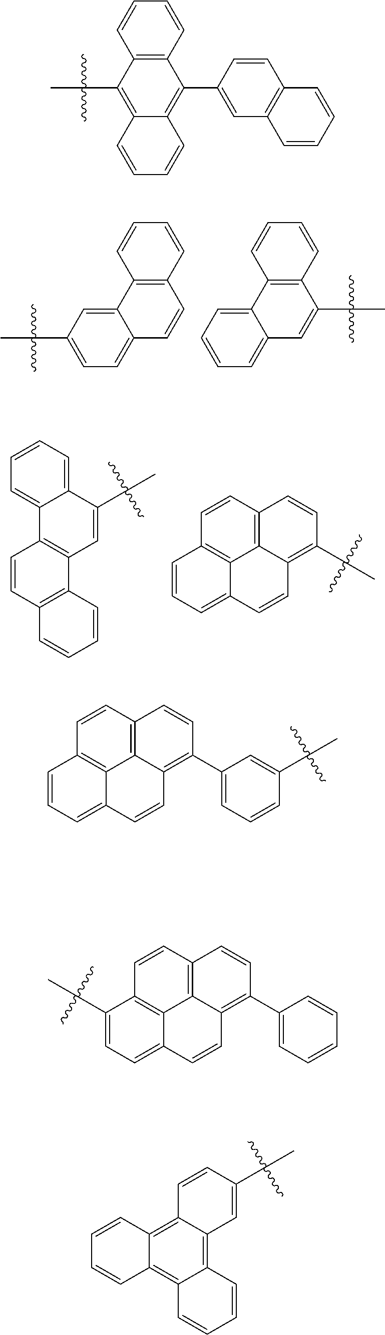

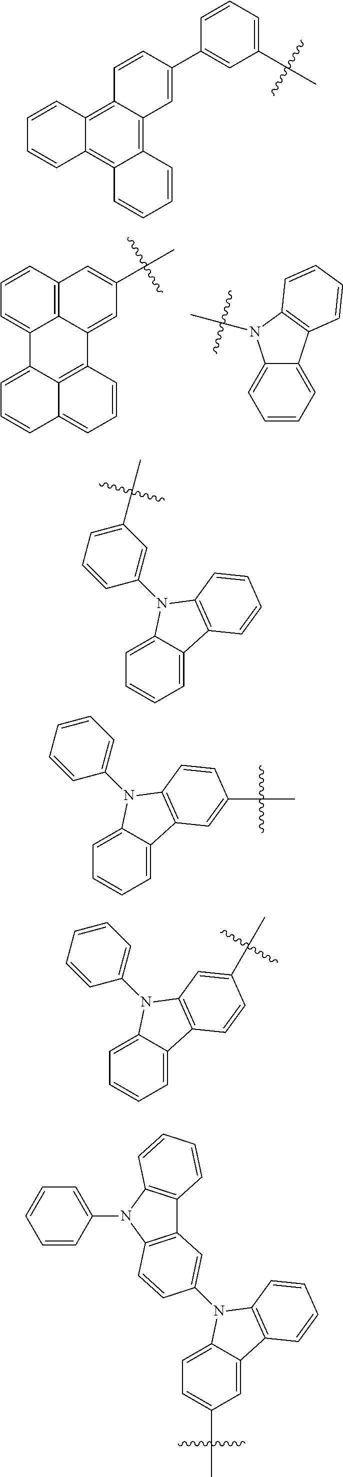

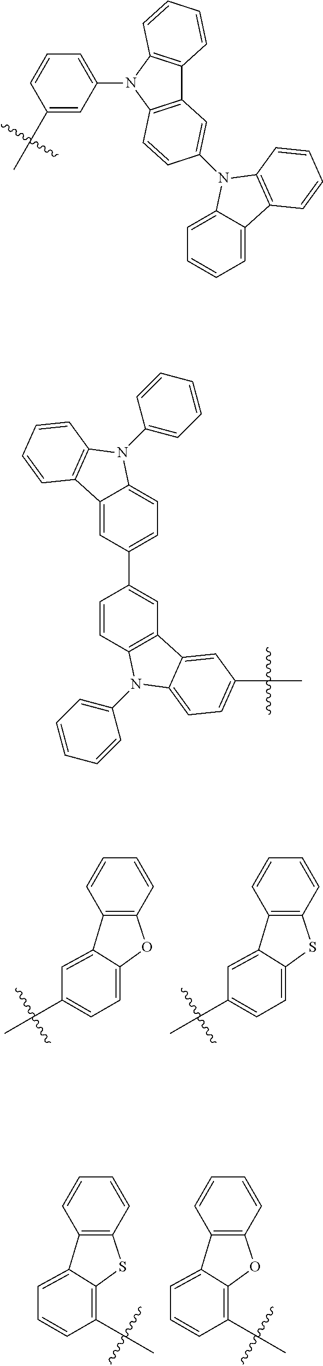 [SMILES: *c1cccc(-n2c3ccccc3c3cc(-n4c5ccccc5c5ccccc54)ccc32)c1 ; *c1ccc2c(c1)c1cc(-c3ccc4c(c3)c3ccccc3n4-c3ccccc3)ccc1n2-c1ccccc1 ; *c1ccc2oc3ccccc3c2c1 ; *c1ccc2sc3ccccc3c2c1 ; *c1cccc2c1sc1ccccc12 ; *c1cccc2c1oc1ccccc12]

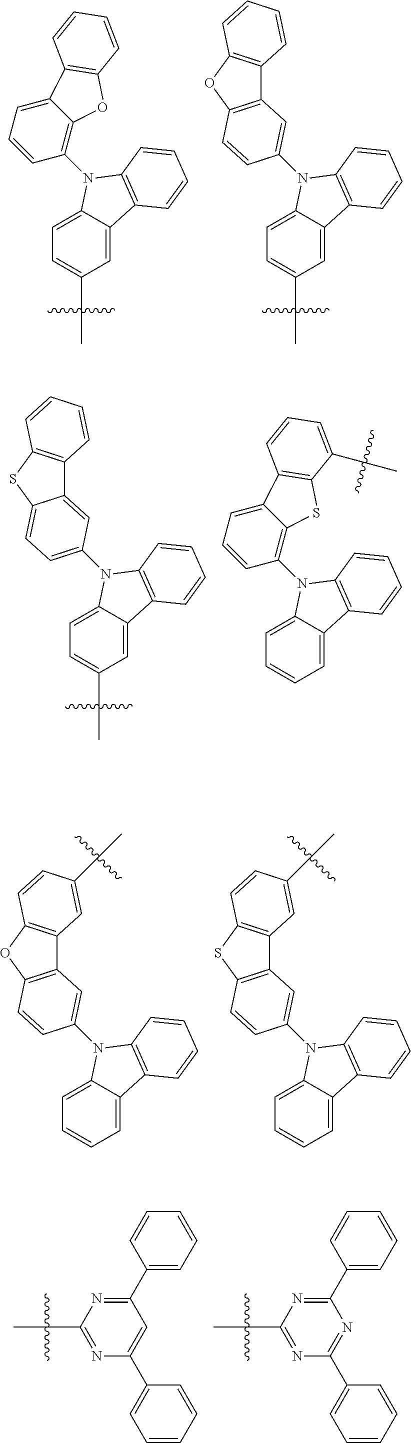

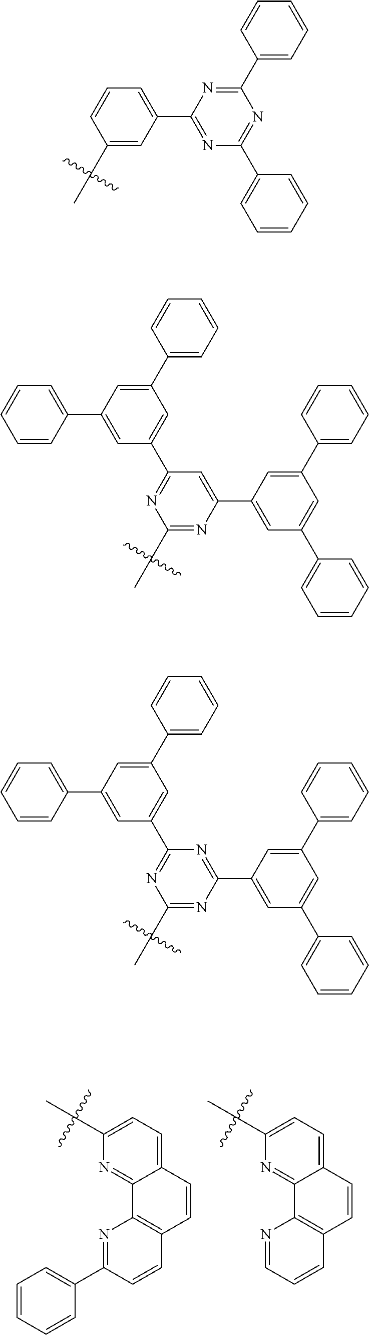 [SMILES: *c1cccc(-c2nc(-c3ccccc3)nc(-c3ccccc3)n2)c1 ; *c1nc(-c2cc(-c3ccccc3)cc(-c3ccccc3)c2)cc(-c2cc(-c3ccccc3)cc(-c3ccccc3)c2)n1 ; *c1nc(-c2cc(-c3ccccc3)cc(-c3ccccc3)c2)nc(-c2cc(-c3ccccc3)cc(-c3ccccc3)c2)n1 ; *c1ccc2ccc3ccc(-c4ccccc4)nc3c2n1 ; *c1ccc2ccc3cccnc3c2n1]

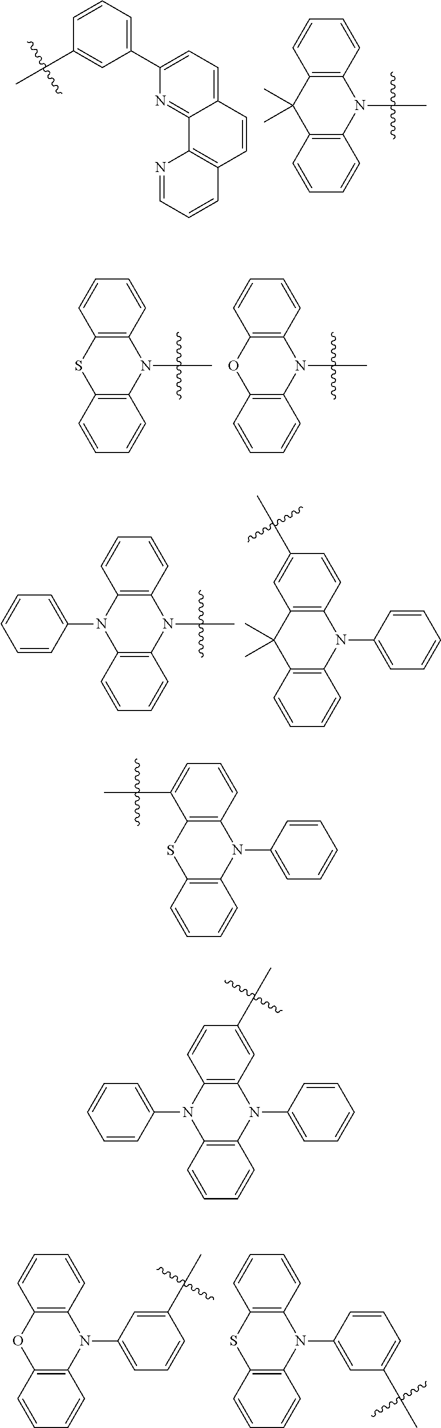

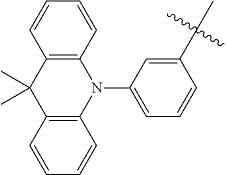 [SMILES: *c1cccc(N2c3ccccc3C(C)(C)c3ccccc32)c1]

When L₁, L₂ are not represented single bond, some preferable arylene group and heterarylene group for L₁ and L₂ are consisting of group represent as:

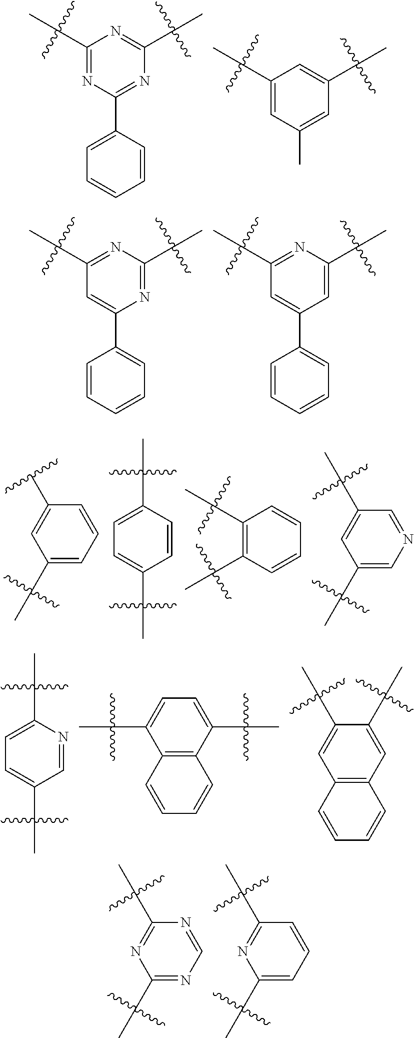

When L₁, L₂ are simultaneously represented single bond and Ar₁, Ar₂ are different, some preferable group of Ar₁ and Ar₂ are consisting of group represent as:

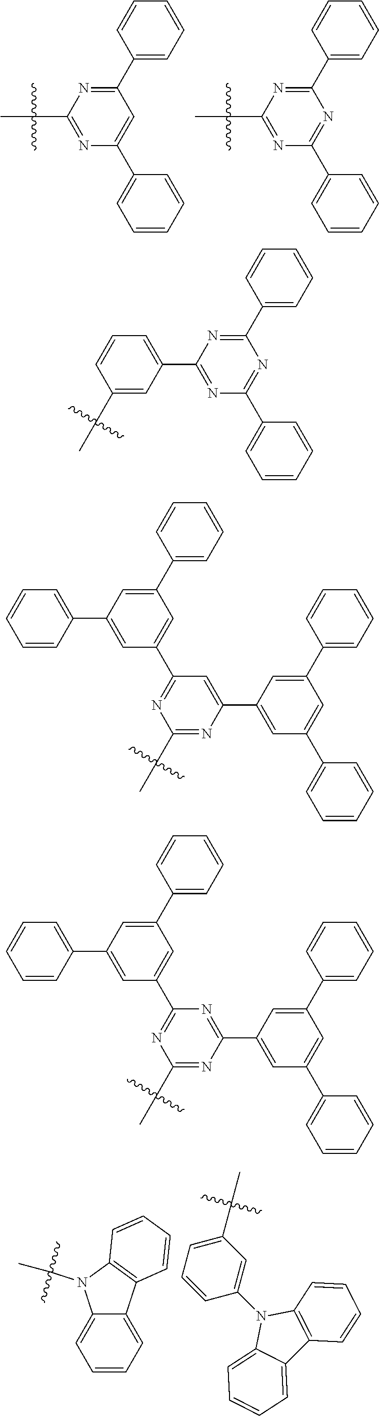

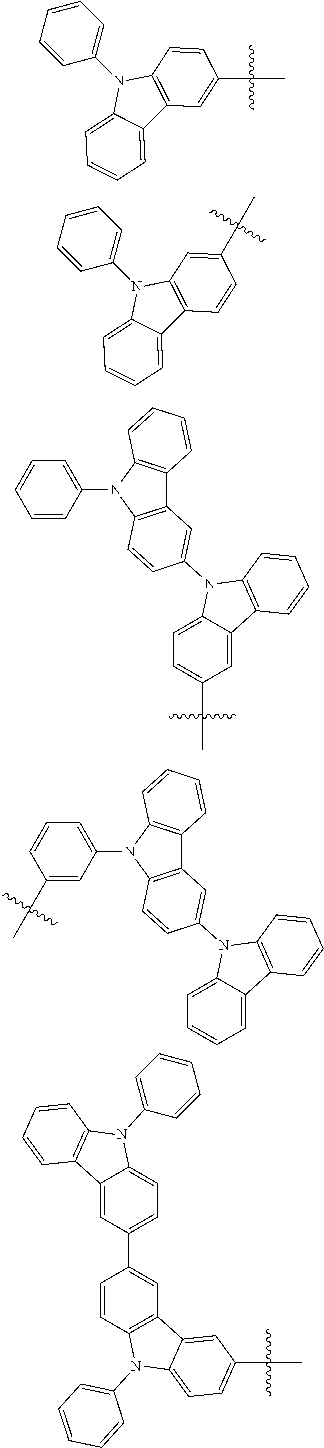

In this embodiment, some phenanthro[9,10-b]tetraphenylene derivative are shown below:

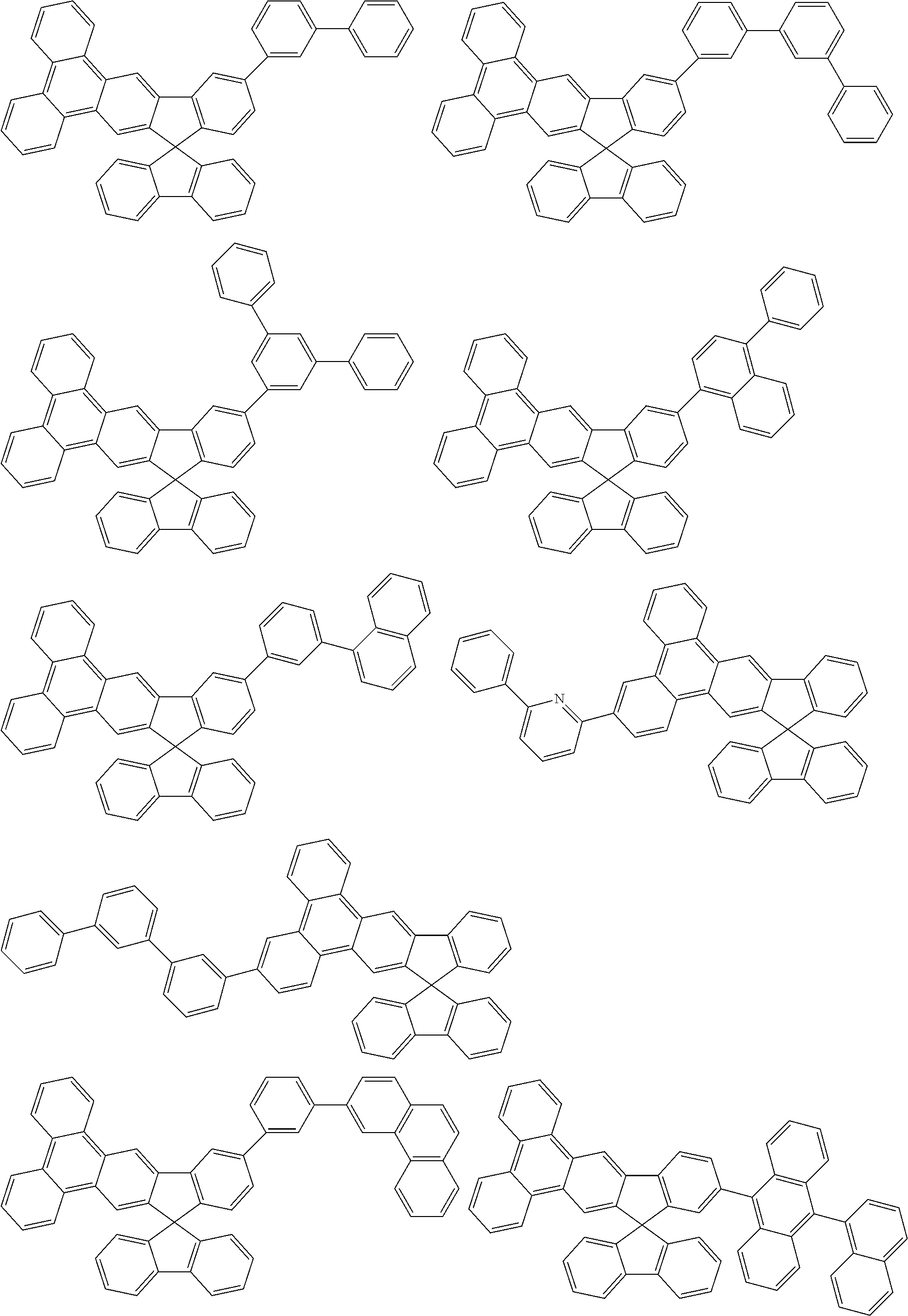

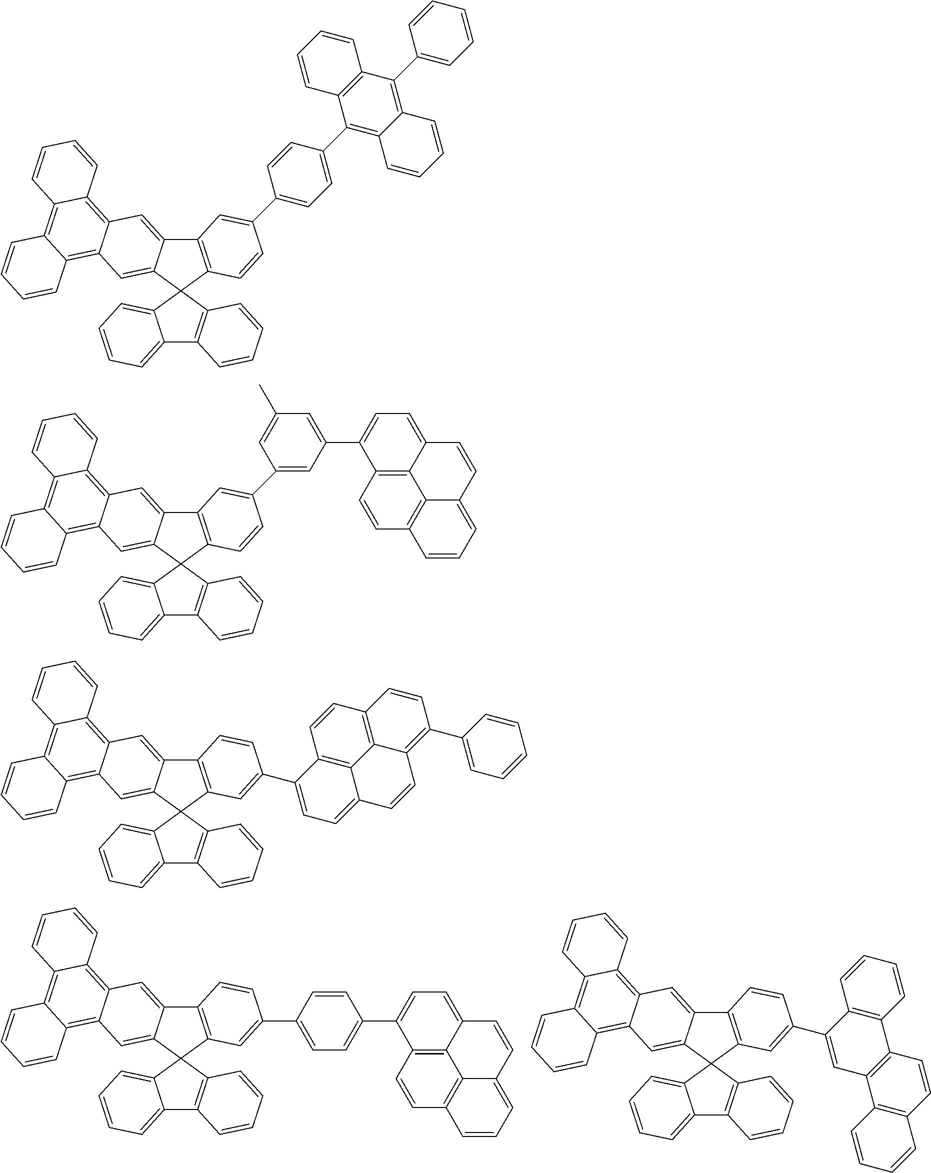

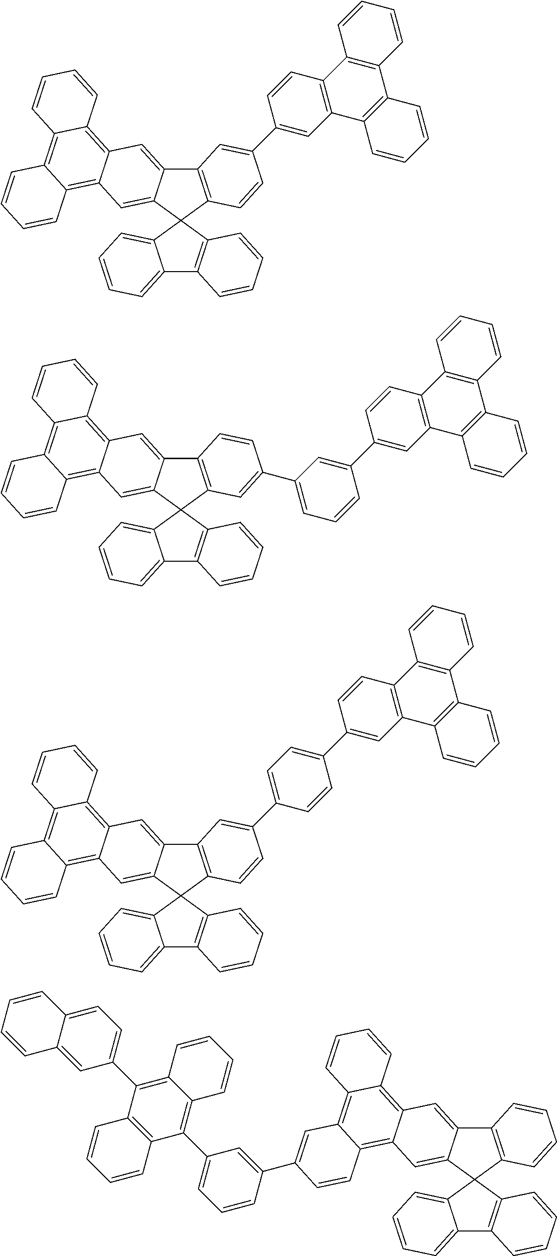

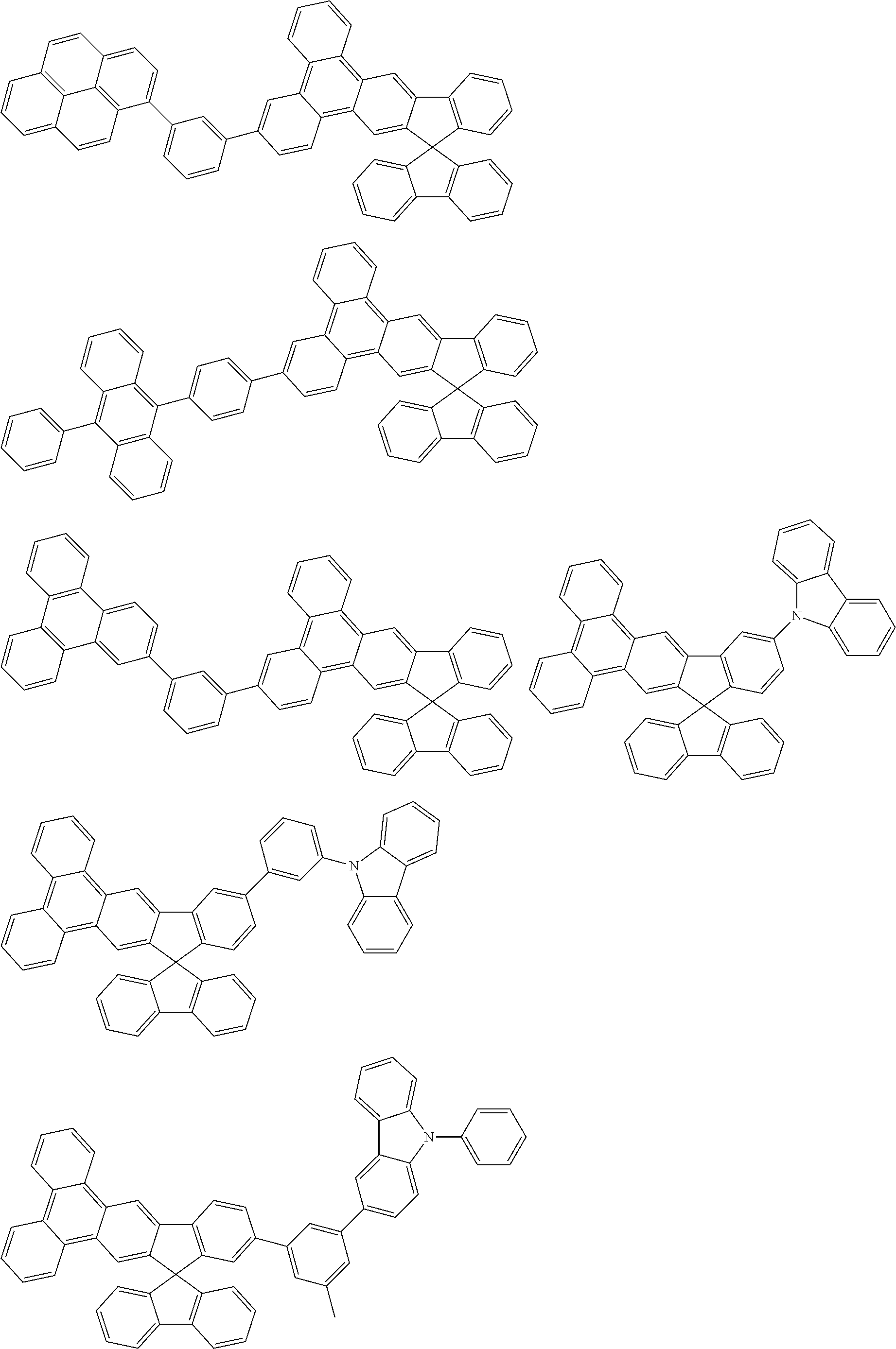

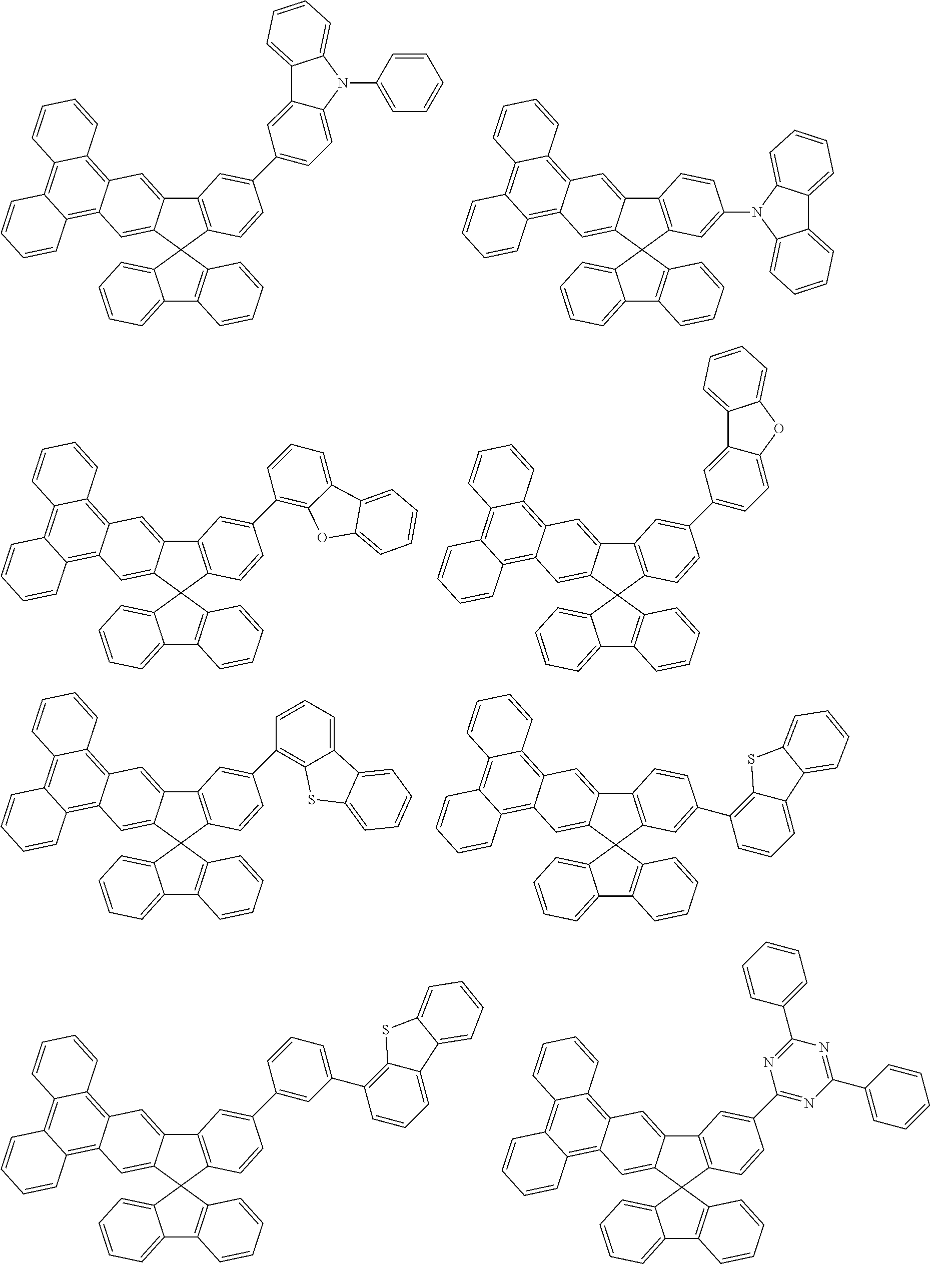

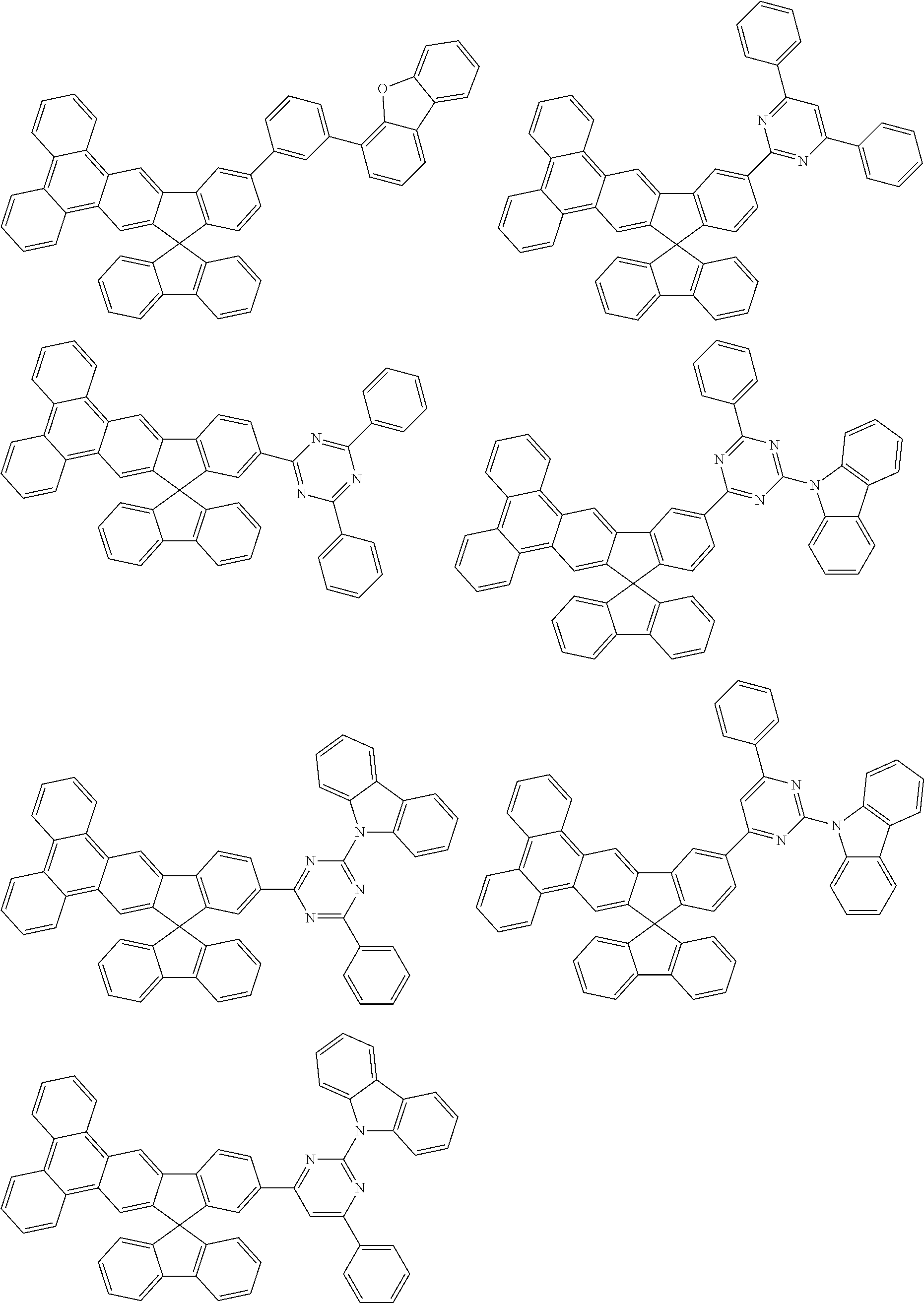

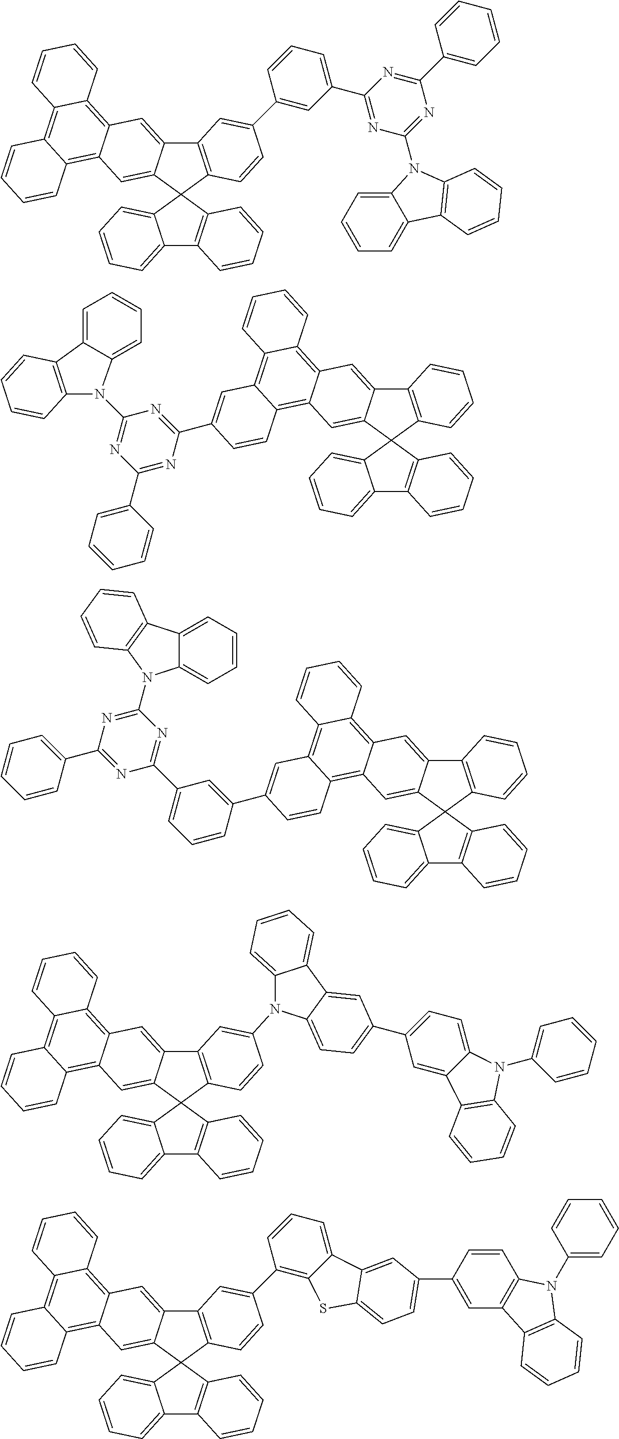

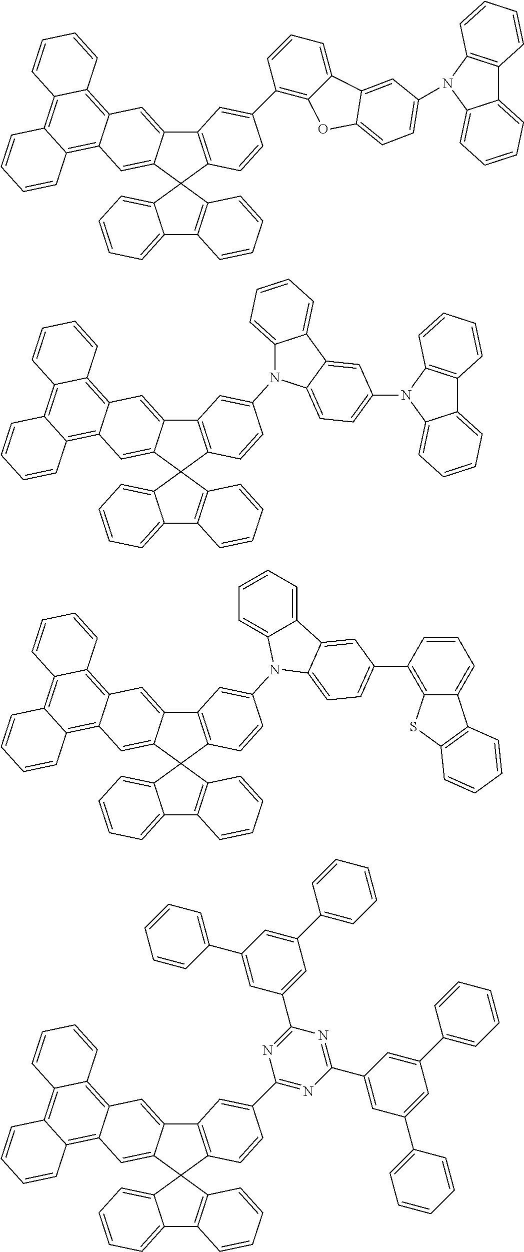

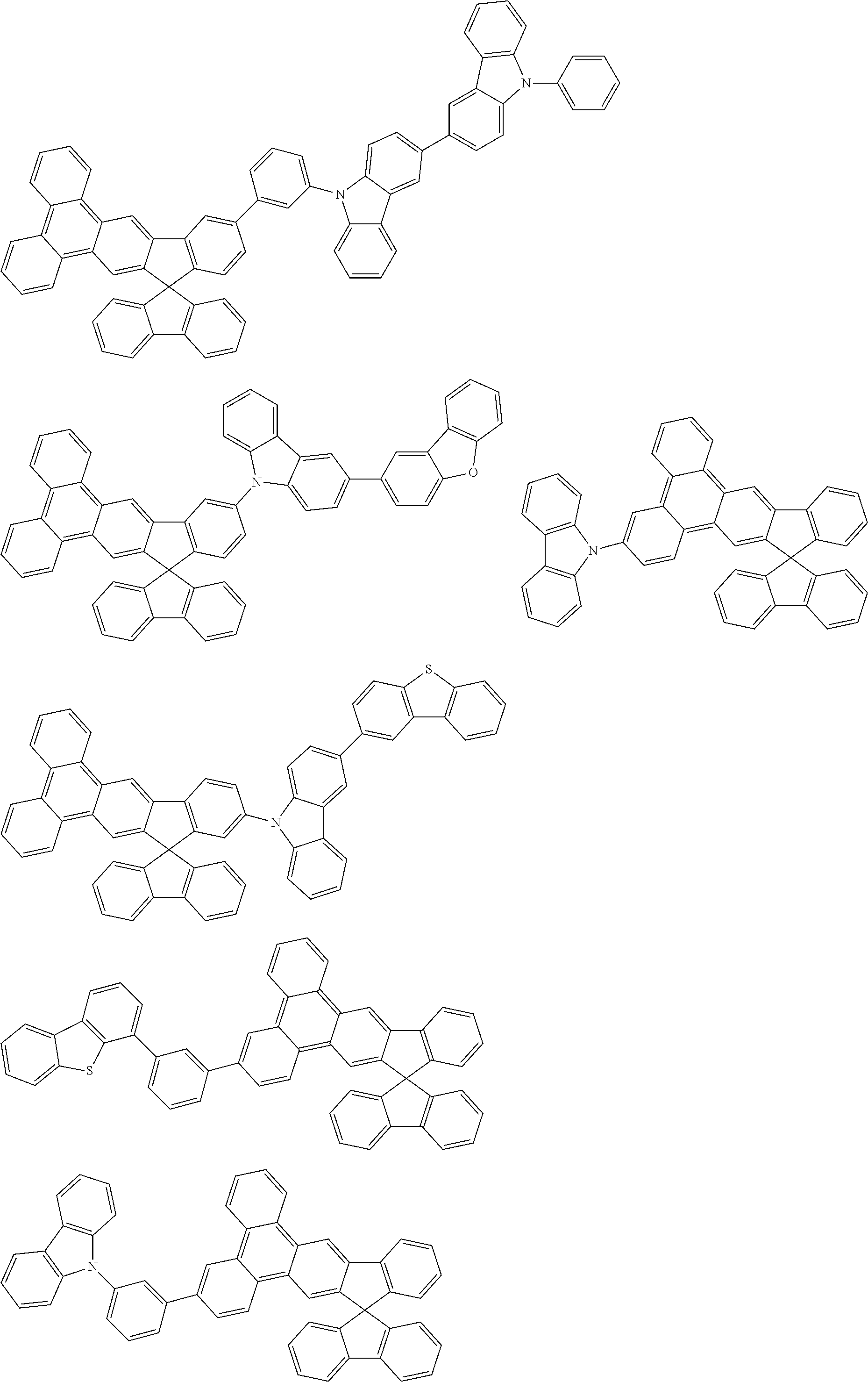

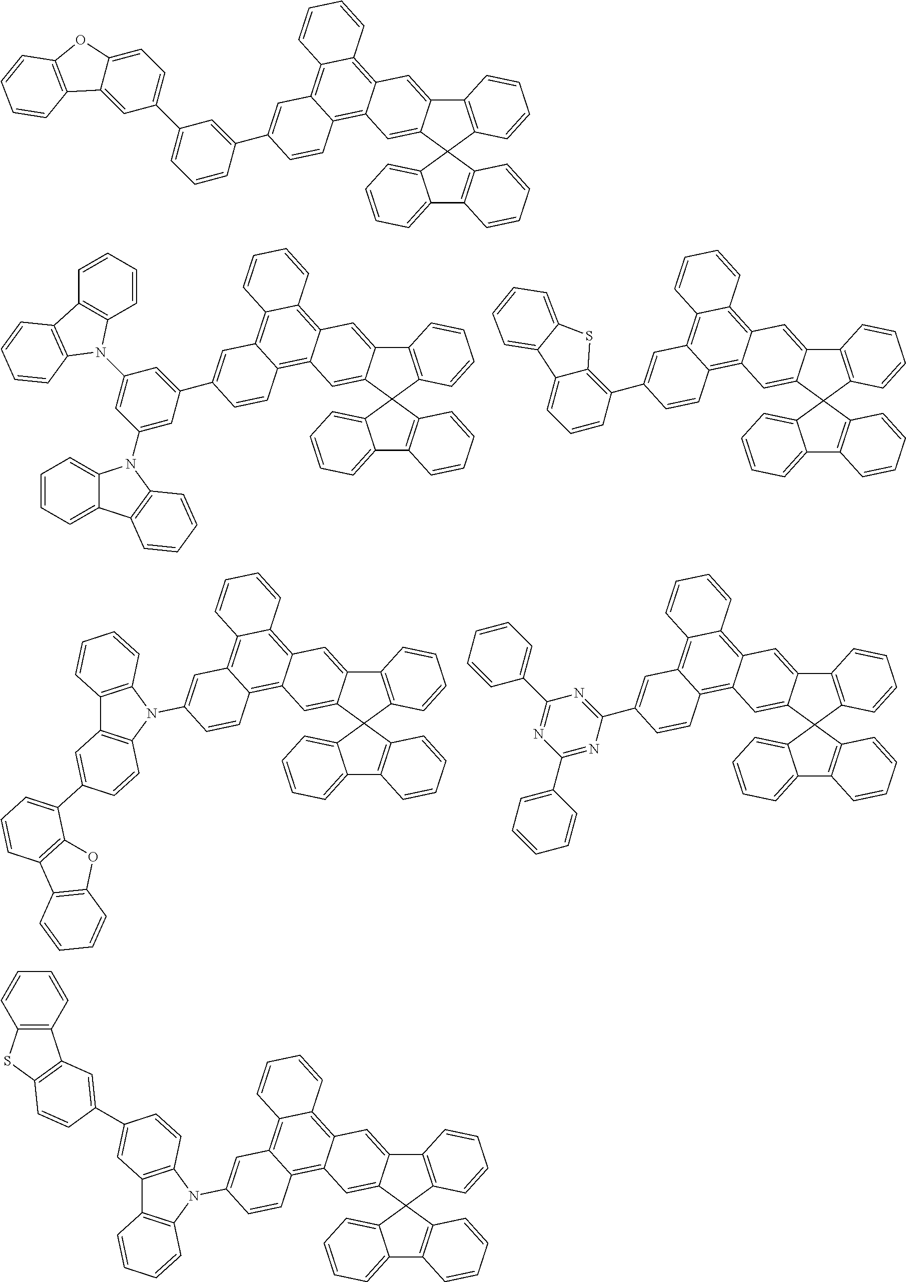

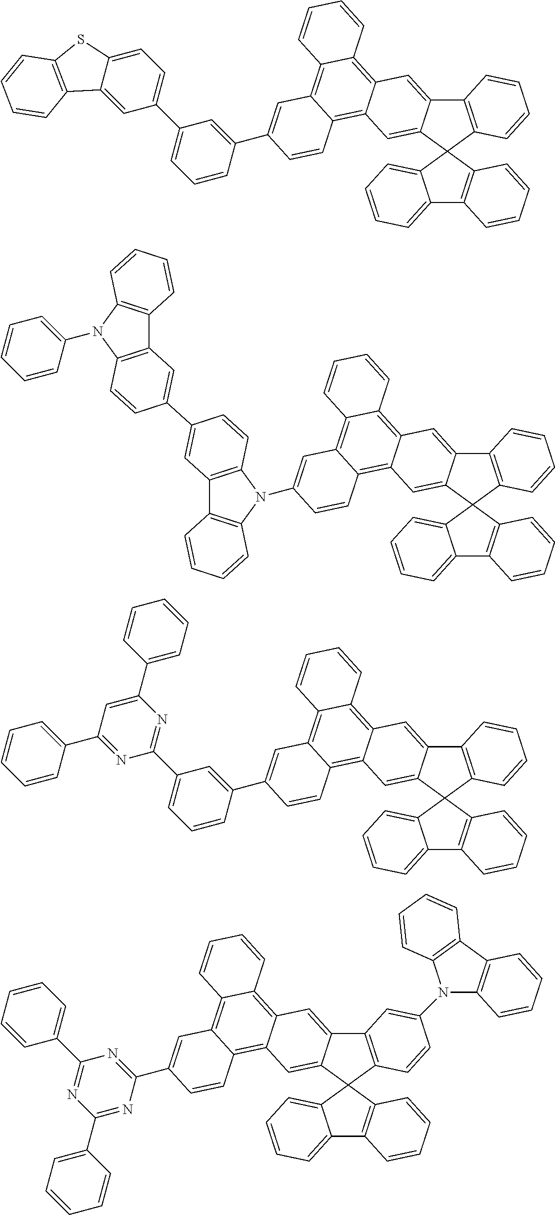

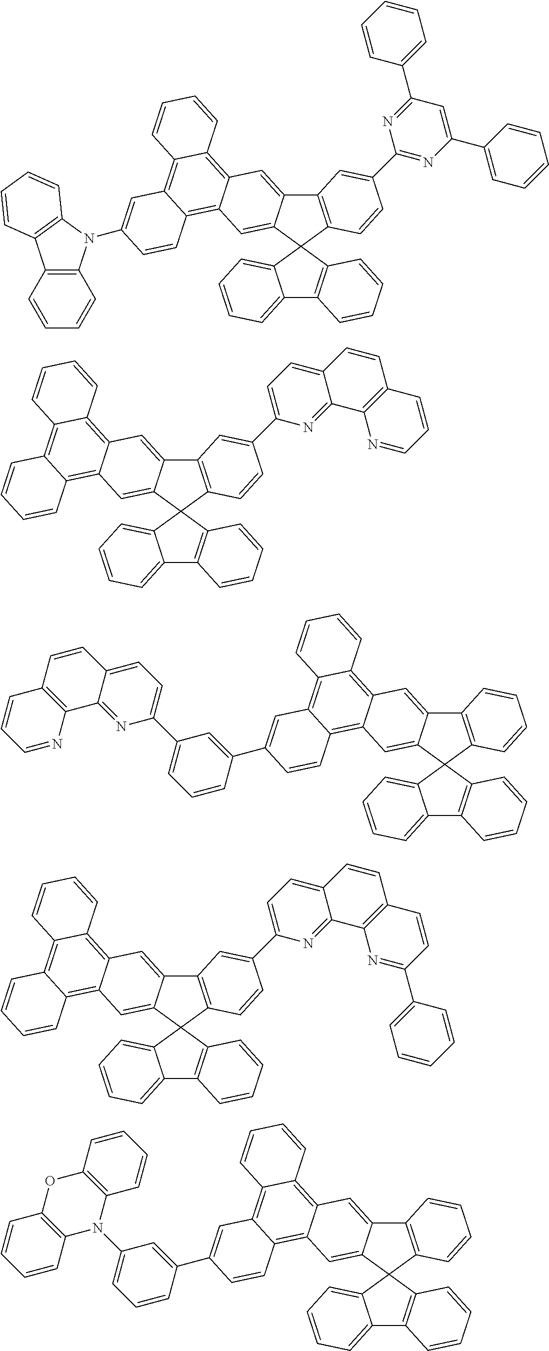

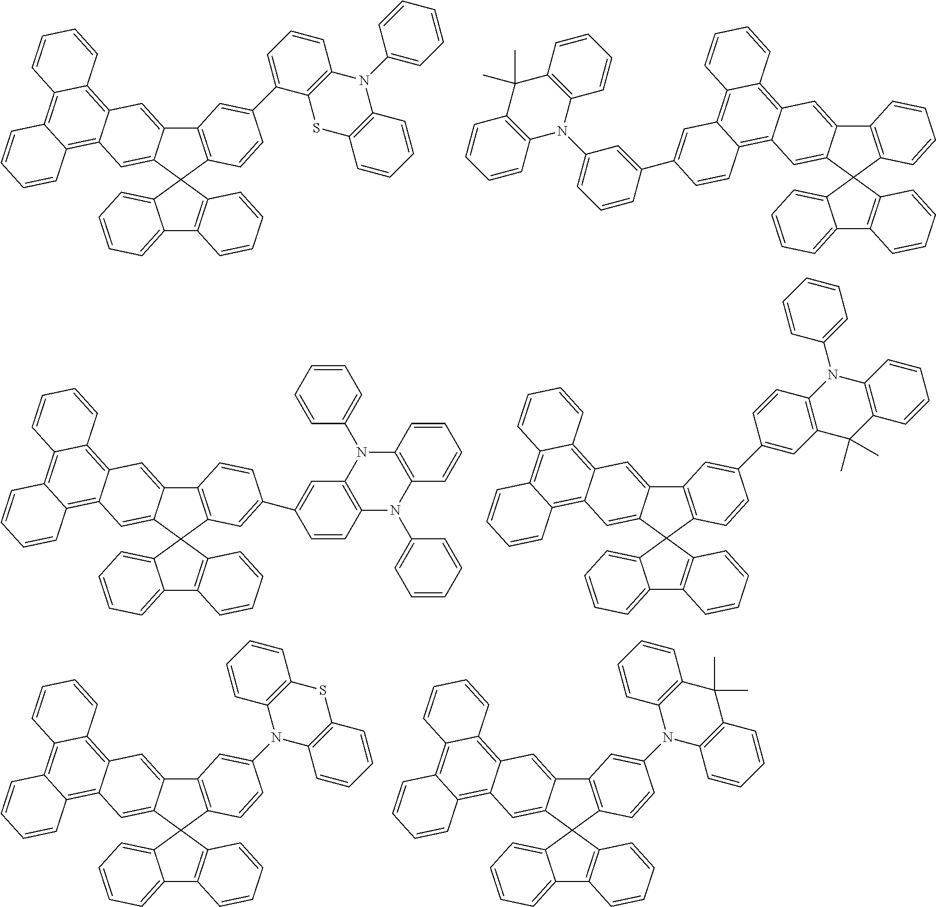

Detailed preparation for the phenanthro[9,10-b]tetraphenylene derivative in the present invention could be clarified by exemplary embodiments, but the present invention is not limited to exemplary embodiments. EXAMPLE Ia˜Ig and EXAMPLE 1˜74 show the preparation for some EXAMPLES of the phenanthro[9,10-b]tetraphenylene derivative in the present invention. EXAMPLE 75 and 79 show the fabrication of organic EL device and I-V-B, half-life time of organic EL device testing report.

### Example Ia

### Synthesis of Intermediate Ia

### Synthesis of 3,6-dibromo-9,9′-spirobifluorene

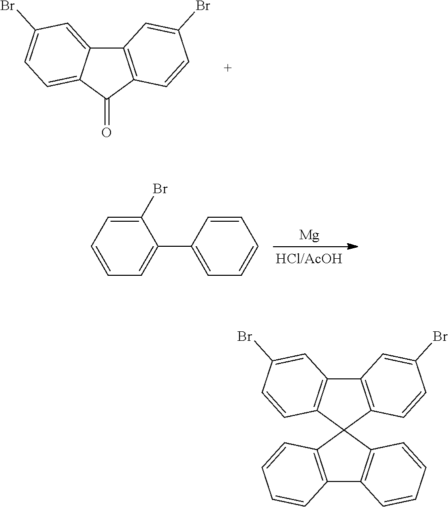

The Grignard reagent was prepared from 7.3 g (300 mmol) of magnesium, 0.5 g of iodine, 46.6 g (200 mmol) of 2-bromobiphenyl, 600 ml of THF and 150 ml of toluene with heating at 70° C. When the magnesium has reacted completely, the mixture was cool to room temperature, and a solution of 67.6 g (200 mmol) of 3,6-dibromo-9H-fluoren-9-one in 500 ml of THF was added dropwise, then the reaction mixture was warmed at 70° C. for 1 hour and then stirred at room temperature overnight. 500 ml of water are added, the solution was extracted with ethyl acetate and water. The organic layer was dried with anhydrous magnesium sulfate and the solvent was evaporated under reduced pressure. The residue was suspended in 700 ml of acetic acid at 40° C. and 5 ml of sulfuric acid was added to the suspension, and the mixture was stirred at 100° C. for a further 4 hours. After cooling, the precipitated solid was filtered off with suction, washed with ethanol. The product was purified by column chromatography to get 37.9 g of product (yield 41%).

### Synthesis of 3-(biphenyl-2-yl)-6-bromo-9,9′-spirobifluorene

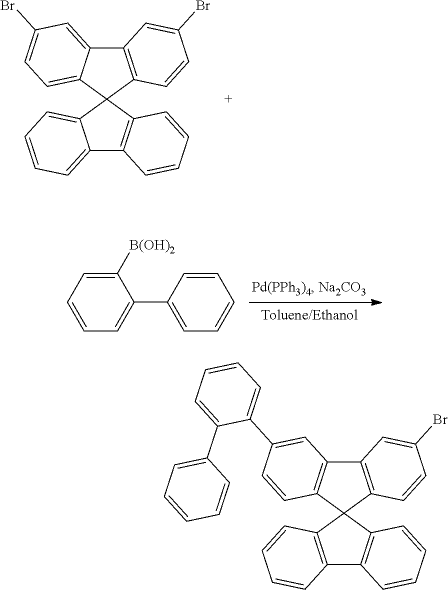

A mixture of 23.1 g (50 mmol) of 3,6-dibromo-9,9′-spirobi fluorene, 9.9 g (50 mmol) of biphenyl-2-ylboronic acid, 2.31 g (2 mmol) of tetrakis(triphenylphosphine)palladium, 75 ml of 2M Na₂CO₃, 150 ml of EtOH and 300 ml toluene was degassed and placed under nitrogen, and then heated at 100° C. for 8 hours. After finishing the reaction, the mixture was allowed to cool to room temperature. The organic layer was extracted with ethyl acetate and water, dried with anhydrous magnesium sulfate, the solvent was removed and the residue was purified by column chromatography on silica (hexane-dichloromethane) to give product 10.7 g (yield 39%) as a white solid.

### Synthesis of 13′-bromospiro[fluorene-9, 10′-indeno[1,2-b]triphenylene]

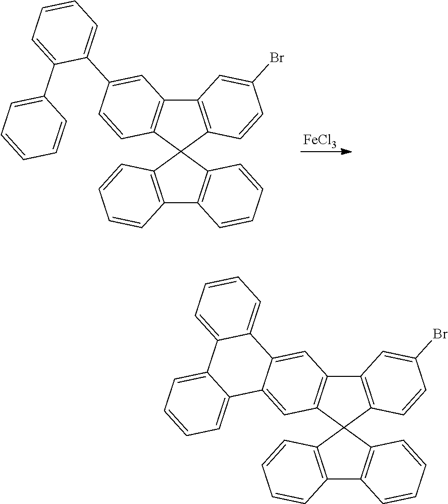

In a 1000 ml three-necked flask that had been degassed and filled with nitrogen, 10.7 g (19.5 mmol) of 3-(biphenyl-2-yl)-6-bromo-9,9′-spirobi[fluorene] was dissolved in anhydrous dichloromethane (450 ml), 16.2 g (100 mmol) iron (III) chloride was then added, and the mixture was stirred 5 minutes. Methanol 100 ml were added to the mixture and the organic layer was separated and the solvent removed in vacuo. The residue was purified by column chromatography on silica (hexane-dichloromethane) afforded a white solid 7.6 g (13.8 mmol, 71%). ¹H NMR (CDCl₃, 400 MHz): chemical shift (ppm) 9.13 (s, 1H), 8.91 (d, J=8.0 Hz, 1H), 8.8 (d, J=8.0 Hz, 1H), 8.54˜8.17 (m, 6H), 7.73˜7.32 (m, 6H), 7.13˜7.10 (m, 3H), 6.82 (d, J=8.0 Hz, 1H), 6.71˜6.63 (m, 2H)

### Example Ib

### Synthesis of Intermediate Ib

### Synthesis of 6-(biphenyl-2-yl)-2-bromo-9,9′-spirobifluorene

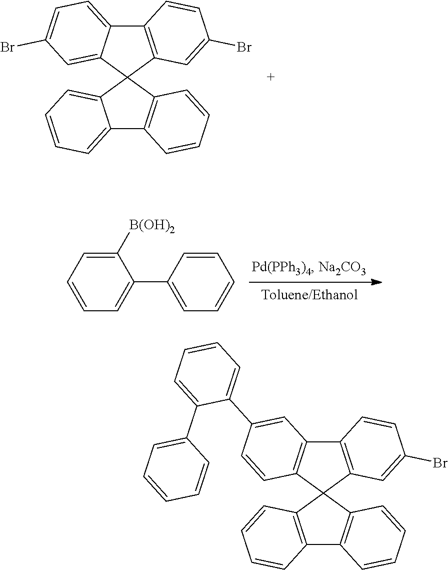

A mixture of 23.1 g (50 mmol) of 2,7-dibromo-9,9′-spirobi fluorene, 9.9 g (50 mmol) of biphenyl-2-ylboronic acid, 2.31 g (2 mmol) of tetrakis(triphenylphosphine)palladium, 75 ml of 2M Na₂CO₃, 150 ml of EtOH and 300 ml toluene was degassed and placed under nitrogen, and then heated at 100° C. for 8 hours. After finishing the reaction, the mixture was allowed to cool to room temperature. The organic layer was extracted with ethyl acetate and water, dried with anhydrous magnesium sulfate, the solvent was removed and the residue was purified by column chromatography on silica (hexane-dichloromethane) to give product 12.3 g (yield 45%) as a white solid.

### Synthesis of 12′-bromospiro[fluorene-9,10′-indeno[1,2-b]triphenylene]

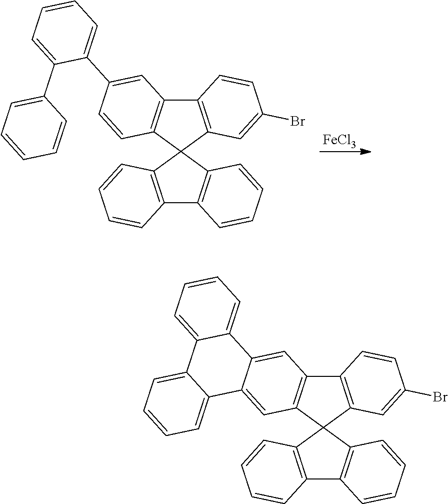

In a 1000 ml three-necked flask that had been degassed and filled with nitrogen, 12.3 g (22.5 mmol) of 6-(biphenyl-2-yl)-2-bromo-9,9′-spirobifluorene was dissolved in anhydrous dichloromethane (700 ml), 29 g (180 mmol) iron (III) chloride was then added, and the mixture was stirred 5 minutes. Methanol 100 ml were added to the mixture and the organic layer was separated and the solvent removed in vacuo. The residue was purified by column chromatography on silica (hexane-dichloromethane) afforded a white solid 8.1 g (14.8 mmol, 66%). ¹H NMR (CDCl₃, 400 MHz):chemical shift (ppm) 9.06 (s, 1H), 8.82 (d, J=8.0 Hz, 1H), 8.63 (d, J=8.0 Hz, 1H), 8.58 (d, J=8.0 Hz, 1H), 8.23 (d, J=8.0 Hz, 1H), 7.95˜7.90 (m, 4H), 7.73˜7.63 (m, 2H), 7.57˜7.52 (m, 2H), 7.45˜7.39 (m, 3H), 7.13˜7.10 (m, 2H), 6.87 (d, J=8.0 Hz, 1H), 6.79˜6.77 (m, 2H)

### Example Ic

### Synthesis of Intermediate Ic

### Synthesis of 6′-methoxyspiro[fluorene-9, 10′-indeno[1,2-b]triphenylene]

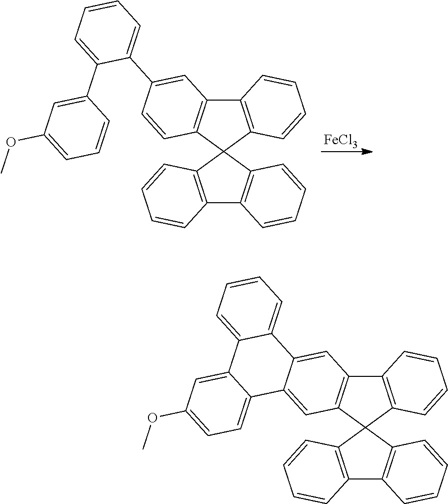

In a 1000 ml three-necked flask that had been degassed and filled with nitrogen, 9.7 g (19.5 mmol) of 2-(5-methoxybiphenyl-2-yl)-9,9′-spirobifluorene was dissolved in anhydrous dichloromethane (450 ml), 16.2 g (100 mmol) iron (III) chloride was then added, and the mixture was stirred 5 minutes. Methanol 100 ml were added to the mixture and the organic layer was separated and the solvent removed in vacuo. The residue was purified by column chromatography on silica (hexane-dichloromethane) afforded a white solid 6.5 g (13.8 mmol, 67%). ¹H NMR (CDCl₃, 400 MHz): chemical shift (ppm) 9.06 (s, 1H), 8.82 (d, J=8.0 Hz, 1H), 8.63˜8.58 (m, 2H), 8.23 (s, 1H), 7.95˜7.90 (m, 4H), 7.79˜7.51 (m, 5H), 7.45˜7.39 (m, 3H), 7.13˜7.10 (m, 2H), 6.87˜6.79 (m, 2H)

### Synthesis of spirofluorene-9,10′-indeno[1,2-b]triphenylen]-6′-ol

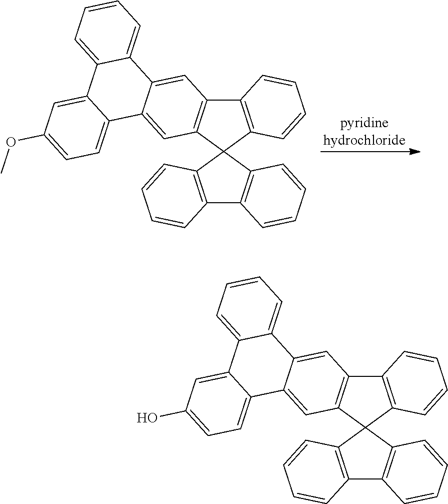 [SMILES: COc1ccc2c(c1)c1ccccc1c1cc3c(cc21)C1(c2ccccc2-c2ccccc21)c1ccccc1-3 ; "pyridinehydrochloride" ; Oc1ccc2c(c1)c1ccccc1c1cc3c(cc21)C1(c2ccccc2-c2ccccc21)c1ccccc1-3]

A mixture of 8.9 g (18 mmol) of 6′-methoxyspiro[fluorene-9,10′-indeno[1,2-b]triphenylene], 31.2 g (270 mmol) of pyridine hydrochloride, was degassed and placed under nitrogen, and then heated at 220° C. for 6 h, the mixture was allowed to cool to room temperature and water was added. The resulting solid was filtered off, washed with water, and dried under high vacuum to give product 7.6 g (16.4 mmol, 87%)

### Synthesis of intermediate Ic

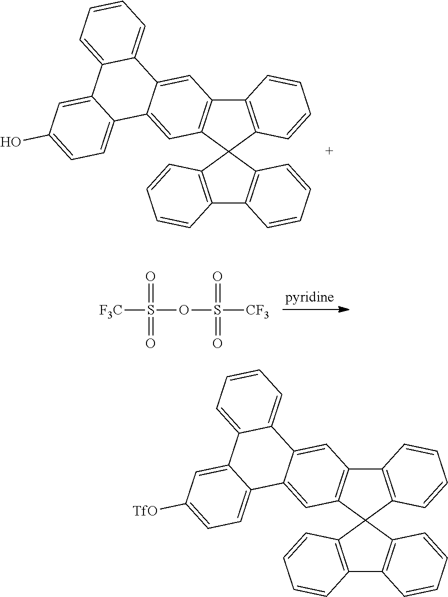

In a 1000 ml three-necked flask that had been degassed and filled with nitrogen, 7.9 g (16.4 mmol) of spiro[fluorene-9,10′-indeno[1,2-b]triphenylen]-6′-ol was dissolved in anhydrous dichloromethane (300 ml), 15 ml pyridine was then added, and the mixture was cooled in an ice salt bath. 5.5 ml (32.8 mmol) trifluoromethanesulfonic anhydride in 50 ml dichloromethane was added dropwise to the solution under nitrogen, the reaction was allowed to proceed for 6 hours and quenched by adding methanol and water. The resulting solid was filtered off, washed with water, methanol and dichloromethane, the residue product was recrystallized from toluene. 6.7 g (11 mmol, 67%) product was obtained.

### Example 1

### Synthesis of 4-(spiro[fluorene-9, 10′-indeno[1,2-b]triphenylene]-13′-yl)dibenzo[b,d]thiophene

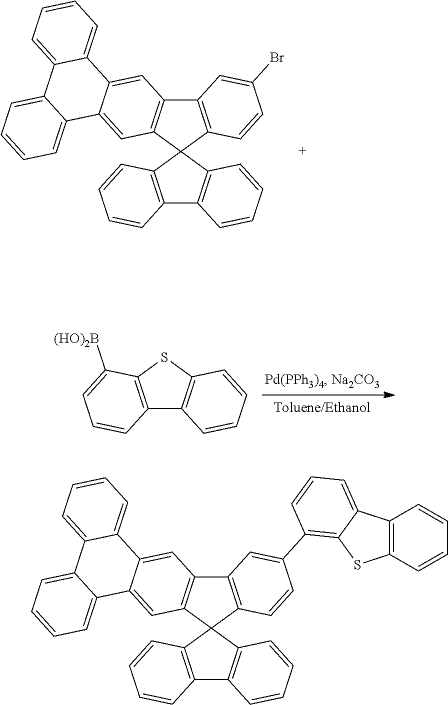 [SMILES: Brc1ccc2c(c1)-c1cc3c4ccccc4c4ccccc4c3cc1C21c2ccccc2-c2ccccc21 ; OB(O)c1cccc2c1sc1ccccc12 ; c1ccc2c(c1)-c1ccccc1C21c2ccc(-c3cccc4c3sc3ccccc34)cc2-c2cc3c4ccccc4c4ccccc4c3cc21]

A mixture of 5.5 g (10.1 mmol) of 13′-bromospiro [fluorene-9,10′-indeno[1,2-b]triphenylene], 2.7 g (12 mmol) of dibenzo[b,d]thiophen-4-ylboronic acid, 0.22 g (0.2 mmol) of tetrakis(triphenylphosphine)palladium, 15 ml of 2M Na₂CO₃, 20 ml of EtOH and 40 ml toluene was degassed and placed under nitrogen, and then heated at 90° C. overnight. After finishing the reaction, the mixture was allowed to cool to room temperature. The solution was extracted with 100 mL of ethyl acetate and 500 ml of water. The organic layer was dried with anhydrous magnesium sulfate and the solvent was evaporated under reduced pressure. The residue was purified by column chromatography on silica (Hx-CH₂Cl₂) to give product 4.1 g (63%). MS (m/z, FAB⁺):648.1

### Example 2

### Synthesis of 4-(3-bromophenyl)dibenzo[b,d]furan

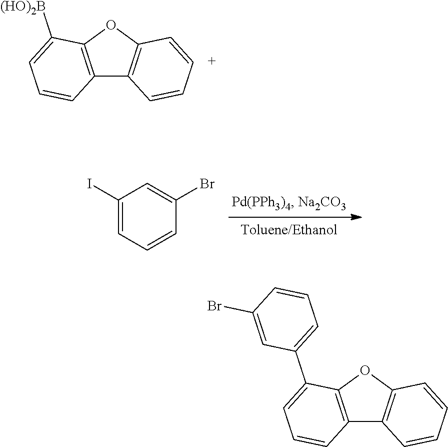 [SMILES: OB(O)c1cccc2c1oc1ccccc12 ; Brc1cccc(I)c1 ; Brc1cccc(-c2cccc3c2oc2ccccc23)c1]

A mixture of 21.2 g (100 mmol) of dibenzo[b,d]furan-4-ylboronic acid, 28.3 g (100 mmol) of 1-bromo-3-iodobenzene, 2.3 g (2 mmol) of tetrakis(triphenylphosphine)palladium, 100 ml of 2M Na₂CO₃, 100 ml of EtOH and 250 ml toluene was degassed and placed under nitrogen, and then heated at 90° C. overnight. After finishing the reaction, the mixture was allowed to cool to room temperature. The solution was extracted with 500 mL of ethyl acetate and 1000 ml of water. The organic layer was dried with anhydrous magnesium sulfate and the solvent was evaporated under reduced pressure. The residue was purified by column chromatography on silica (Hx) to give product 20 g (63%).

### Synthesis of 2-(3-(dibenzo[b,d]furan-4-yl)phenyl)-4,4,5,5-tetramethyl-1,3,2-dioxaborolane

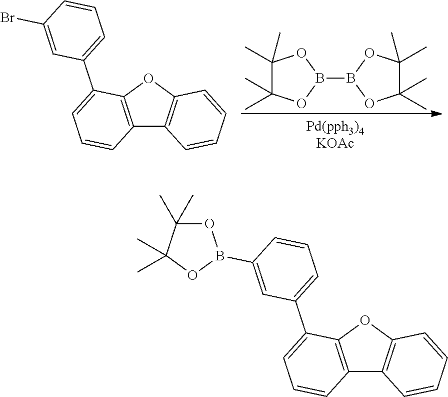

A mixture of 20 g (61.9 mmol) of 4-(3-bromophenyl)dibenzo [b,d]furan, 19 g (75 mmol) of bis(pinacolato)diboron, 1.4 (1.2 mmol) of tetrakis(triphenylphosphine)palladium, 9.1 g (93 mmol) of potassium acetate, and 600 ml of 1,4-dioxane was degassed and placed under nitrogen, and then heated at 90° C. for 16 h. After finishing the reaction, the mixture was allowed to cool to room temperature. The organic layer was extracted with ethyl acetate and water, dried with anhydrous magnesium sulfate, the solvent was removed and the product was purified by column using a mixture of hexanes and dichloromethane as eluent to get 18.3 g of light yellow product (yield 80%).

### Synthesis of 4-(3-(spiro[fluorene-9, 10′-indeno[1,2-b]triphenylene]-13′-yl)phenyl)dibenzo[b, d]furan

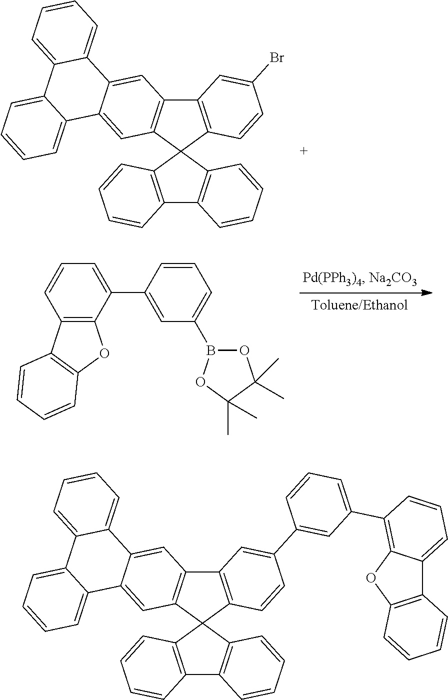

A mixture of 5.5 g (10.1 mmol) of 13′-bromospiro [fluorene-9,10′-indeno[1,2-b]triphenylene], 4.4 g (12 mmol) of 2-(3-(dibenzo[b,d]furan-4-yl)phenyl)-4,4,5,5-tetramethyl-1,3,2-dioxaborolane, 0.22 g (0.2 mmol) of tetrakis(triphenylphosphine)palladium, 15 ml of 2M Na₂CO₃, 20 ml of EtOH and 40 ml toluene was degassed and placed under nitrogen, and then heated at 90° C. overnight. After finishing the reaction, the mixture was allowed to cool to room temperature. The solution was extracted with 100 mL of ethyl acetate and 500 ml of water. The organic layer was dried with anhydrous magnesium sulfate and the solvent was evaporated under reduced pressure. The residue was purified by column chromatography on silica (Hx-CH₂Cl₂) to give product 4.9 g (69%). MS (m/z, FAB⁺):708.9

### Example 3

### Synthesis of N,N-dip-tolylspiro[fluorene-9,10′-indeno[1,2-b]triphenylen]-13′-amine

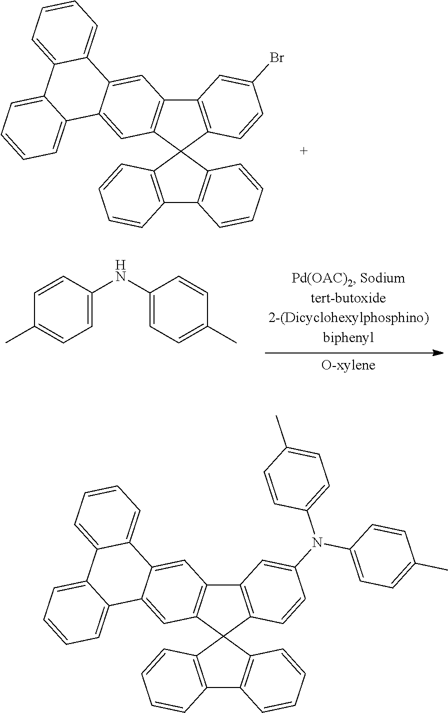

A mixture of 5.5 g (10.1 mmol) 13′-bromospiro[fluorene-9,10′-indeno[1,2-b]triphenylene], 3 g (15.1 mmol) of dip-tolylamine, 0.05 g (0.2 mmol) of palladium(II)acetate, 0.15 g (0.4 mmol) of 2-(dicyclohexylphosphino)biphenyl, 2 g (20 mmol) of sodium tert-butoxide and 100 ml of toluene was refluxed under nitrogen overnight. After finishing the reaction, the solution was filtered at 100° C., to receive the filtrate, and the filtrate was added to 1 L MeOH, while stirring and the precipitated product was filtered off with suction. To give 4.2 g (yield 63%) of yellow product which was recrystallized from toluene. MS (m/z, FAB⁺):661.7

### Example 4

### Synthesis of N,N-di(biphenyl-4-yl)spiro[fluorene-9,10′-indeno[1,2-b]triphenylen]-12′-amine

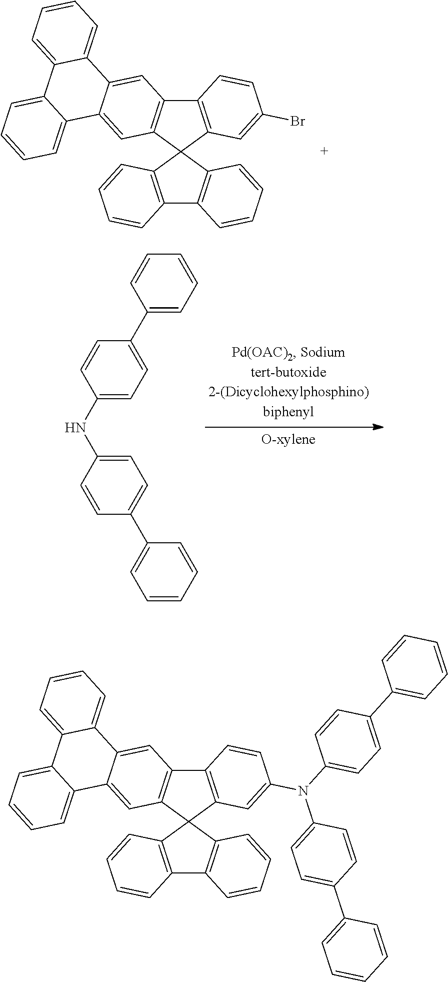

A mixture of 5.5 g (10.1 mmol) 12′-bromospiro[fluorene-9,10′-indeno[1,2-b]triphenylene], 4.9 g (15.1 mmol) of dibiphenyl-4-ylamine, 0.05 g (0.2 mmol) of palladium(II)acetate, 0.15 g (0.4 mmol) of 2-(dicyclohexyl phosphino)biphenyl, 2 g (20 mmol) of sodium tert-butoxide and 100 ml of toluene was refluxed under nitrogen overnight. After finishing the reaction, than cooled to room temperature. The crystalline precipitates was filtrated and rinsed with 50 ml of MeOH and 100 ml of dichloromethane. The product was purified by sublimation to get 2.9 g of product. (yield 37%). MS (m/z, FAB⁺):786.1

### Example 5˜31

The following compounds are synthesized analogously.

| | Inter- | | | |
|---|---|---|---|---|
| | me- | | | |
| Ex. | diate I | Intermediate II | Product | Yield |
| 5 | Inter- me- diate Ia | 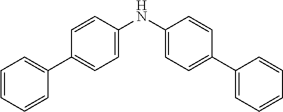 [SMILES: c1ccc(-c2ccc(Nc3ccc(-c4ccccc4)cc3)cc2)cc1] | 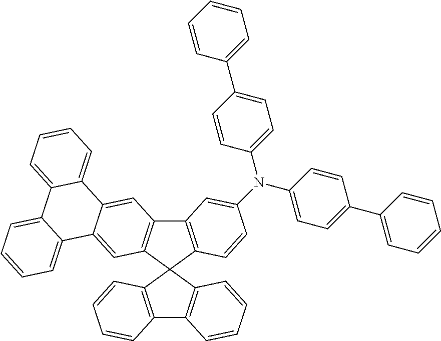 [SMILES: c1ccc(-c2ccc(N(c3ccc(-c4ccccc4)cc3)c3ccc4c(c3)-c3cc5c6ccccc6c6ccccc6c5cc3C43c4ccccc4-c4ccccc43)cc2)cc1] | 36% |
| 6 | Inter- me- diate Ib | 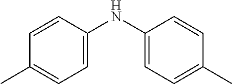 | 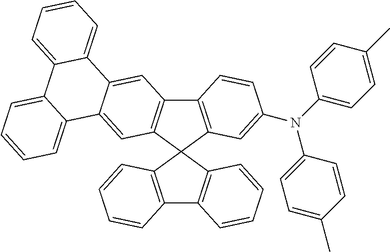 [SMILES: Cc1ccc(N(c2ccc(C)cc2)c2ccc3c(c2)C2(c4ccccc4-c4ccccc42)c2cc4c5ccccc5c5ccccc5c4cc2-3)cc1] | 43% |
| 7 | Inter- me- diate Ia | 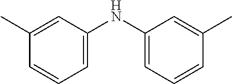 | 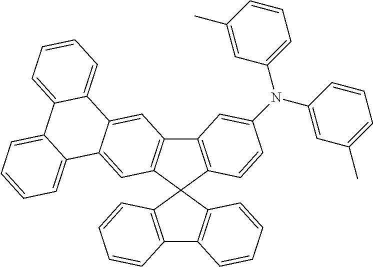 [SMILES: Cc1cccc(N(c2cccc(C)c2)c2ccc3c(c2)-c2cc4c5ccccc5c5ccccc5c4cc2C32c3ccccc3-c3ccccc32)c1] | 45% |
| 8 | Inter- me- diate Ib | 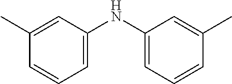 | 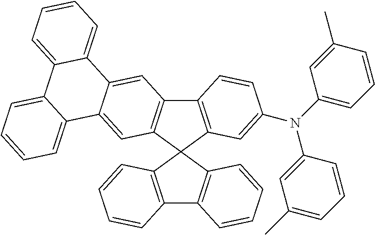 [SMILES: Cc1cccc(N(c2cccc(C)c2)c2ccc3c(c2)C2(c4ccccc4-c4ccccc42)c2cc4c5ccccc5c5ccccc5c4cc2-3)c1] | 41% |
| 9 | Inter- me- diate Ia | 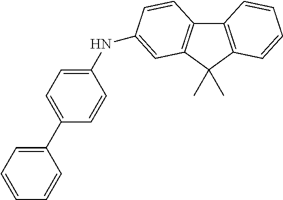 [SMILES: CC1(C)c2ccccc2-c2ccc(Nc3ccc(-c4ccccc4)cc3)cc21] | 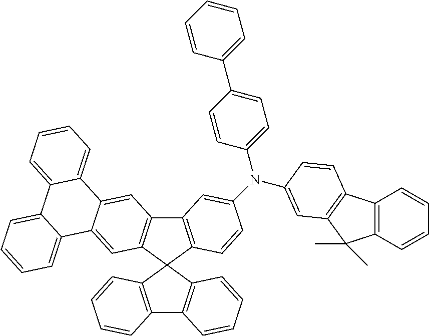 [SMILES: CC1(C)c2ccccc2-c2ccc(N(c3ccc(-c4ccccc4)cc3)c3ccc4c(c3)-c3cc5c6ccccc6c6ccccc6c5cc3C43c4ccccc4-c4ccccc43)cc21] | 55% |
| 10 | Inter- me- diate Ib | 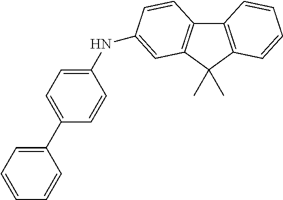 [SMILES: CC1(C)c2ccccc2-c2ccc(Nc3ccc(-c4ccccc4)cc3)cc21] | 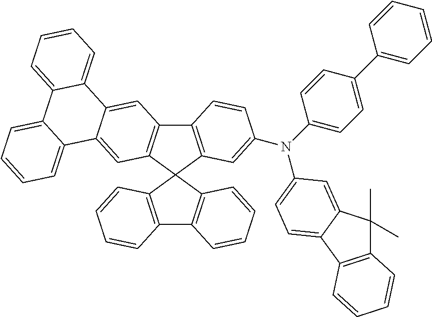 [SMILES: CC1(C)c2ccccc2-c2ccc(N(c3ccc(-c4ccccc4)cc3)c3ccc4c(c3)C3(c5ccccc5-c5ccccc53)c3cc5c6ccccc6c6ccccc6c5cc3-4)cc21] | 47% |
| 11 | Inter- me- diate Ia | 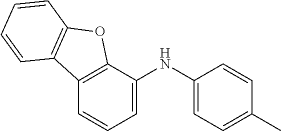 [SMILES: Cc1ccc(Nc2cccc3c2oc2ccccc23)cc1] | 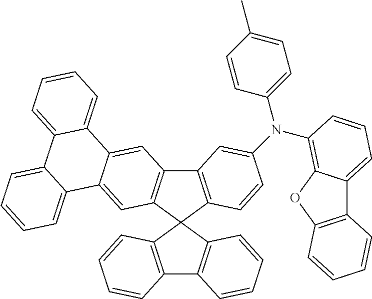 [SMILES: Cc1ccc(N(c2ccc3c(c2)-c2cc4c5ccccc5c5ccccc5c4cc2C32c3ccccc3-c3ccccc32)c2cccc3c2oc2ccccc23)cc1] | 33% |
| 12 | Inter- me- diate Ib | 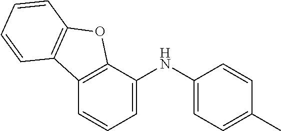 [SMILES: Cc1ccc(Nc2cccc3c2oc2ccccc23)cc1] | 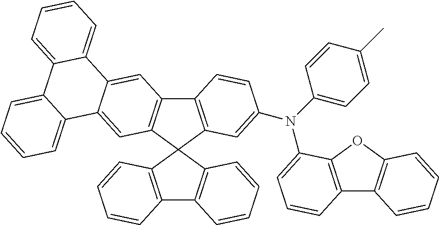 [SMILES: Cc1ccc(N(c2ccc3c(c2)C2(c4ccccc4-c4ccccc42)c2cc4c5ccccc5c5ccccc5c4cc2-3)c2cccc3c2oc2ccccc23)cc1] | 39% |
| 13 | Inter- me- diate Ia | 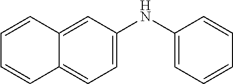 [SMILES: c1ccc(Nc2ccc3ccccc3c2)cc1] | 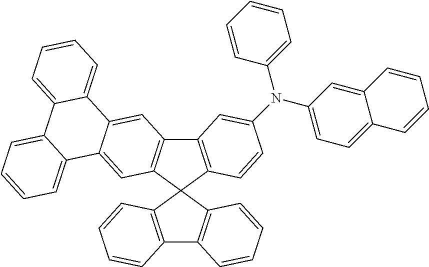 [SMILES: c1ccc(N(c2ccc3c(c2)-c2cc4c5ccccc5c5ccccc5c4cc2C32c3ccccc3-c3ccccc32)c2ccc3ccccc3c2)cc1] | 49% |
| 14 | Inter- me- diate Ib | 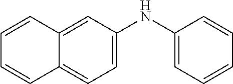 [SMILES: c1ccc(Nc2ccc3ccccc3c2)cc1] | 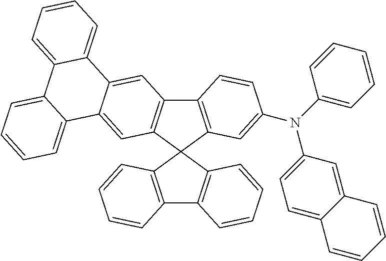 [SMILES: c1ccc(N(c2ccc3c(c2)C2(c4ccccc4-c4ccccc42)c2cc4c5ccccc5c5ccccc5c4cc2-3)c2ccc3ccccc3c2)cc1] | 41% |
| 15 | Inter- me- diate Ia | 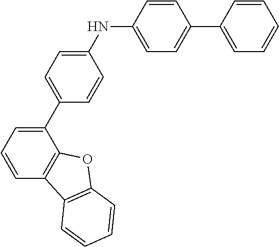 [SMILES: c1ccc(-c2ccc(Nc3ccc(-c4cccc5c4oc4ccccc45)cc3)cc2)cc1] | 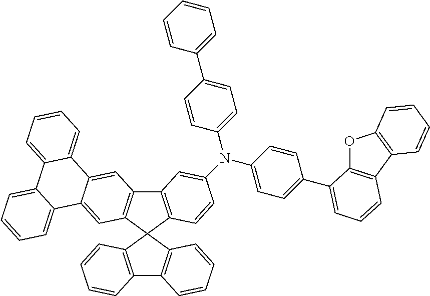 [SMILES: c1ccc(-c2ccc(N(c3ccc(-c4cccc5c4oc4ccccc45)cc3)c3ccc4c(c3)-c3cc5c6ccccc6c6ccccc6c5cc3C43c4ccccc4-c4ccccc43)cc2)cc1] | 56% |
| 16 | Inter- me- diate Ib | 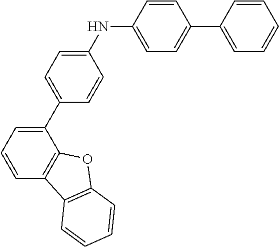 [SMILES: c1ccc(-c2ccc(Nc3ccc(-c4cccc5c4oc4ccccc45)cc3)cc2)cc1] | 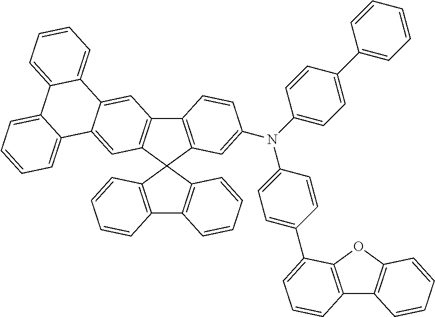 [SMILES: c1ccc(-c2ccc(N(c3ccc(-c4cccc5c4oc4ccccc45)cc3)c3ccc4c(c3)C3(c5ccccc5-c5ccccc53)c3cc5c6ccccc6c6ccccc6c5cc3-4)cc2)cc1] | 61% |
| 17 | Inter- me- diate Ia | 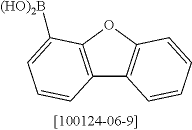 [SMILES: OB(O)c1cccc2c1oc1ccccc12] | 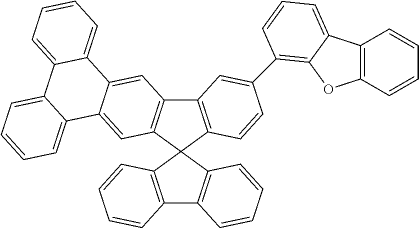 [SMILES: c1ccc2c(c1)-c1ccccc1C21c2ccc(-c3cccc4c3oc3ccccc34)cc2-c2cc3c4ccccc4c4ccccc4c3cc21] | 69% |
| 18 | Inter- me- diate Ia | 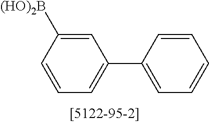 [SMILES: OB(O)c1cccc(-c2ccccc2)c1] | 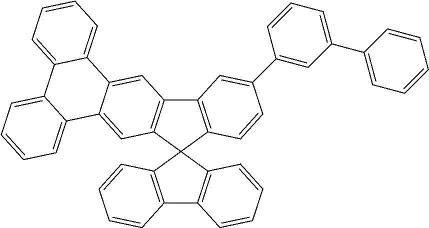 [SMILES: c1ccc(-c2cccc(-c3ccc4c(c3)-c3cc5c6ccccc6c6ccccc6c5cc3C43c4ccccc4-c4ccccc43)c2)cc1] | 61% |
| 19 | Inter- me- diate Ia | 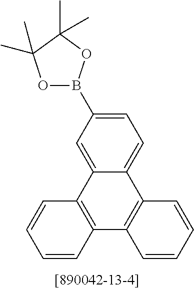 [SMILES: CC1(C)OB(c2ccc3c4ccccc4c4ccccc4c3c2)OC1(C)C] | 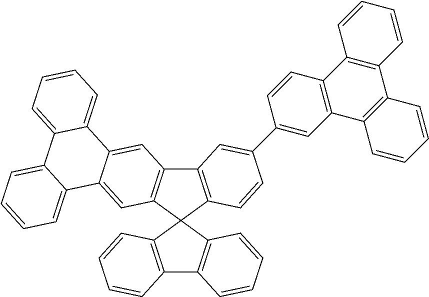 [SMILES: c1ccc2c(c1)-c1ccccc1C21c2ccc(-c3ccc4c5ccccc5c5ccccc5c4c3)cc2-c2cc3c4ccccc4c4ccccc4c3cc21] | 73% |
| 20 | Inter- me- diate Ia | 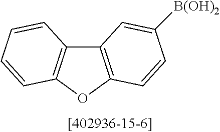 [SMILES: OB(O)c1ccc2oc3ccccc3c2c1] | 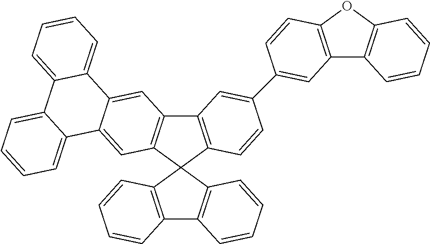 [SMILES: c1ccc2c(c1)-c1ccccc1C21c2ccc(-c3ccc4oc5ccccc5c4c3)cc2-c2cc3c4ccccc4c4ccccc4c3cc21] | 64% |
| 21 | Inter- me- diate Ia | 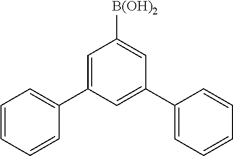 [SMILES: OB(O)c1cc(-c2ccccc2)cc(-c2ccccc2)c1] | 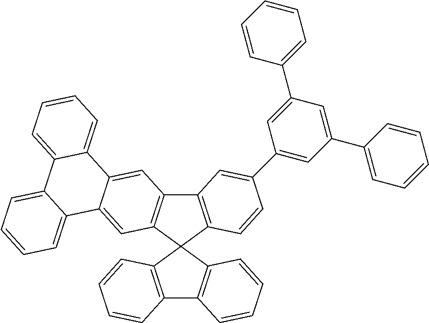 [SMILES: c1ccc(-c2cc(-c3ccccc3)cc(-c3ccc4c(c3)-c3cc5c6ccccc6c6ccccc6c5cc3C43c4ccccc4-c4ccccc43)c2)cc1] | 45% |
| 22 | Inter- me- diate Ia | 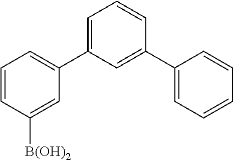 [SMILES: OB(O)c1cccc(-c2cccc(-c3ccccc3)c2)c1] | 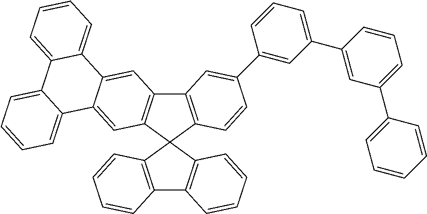 [SMILES: c1ccc(-c2cccc(-c3cccc(-c4ccc5c(c4)-c4cc6c7ccccc7c7ccccc7c6cc4C54c5ccccc5-c5ccccc54)c3)c2)cc1] | 46% |
| 23 | Inter- me- diate Ia | 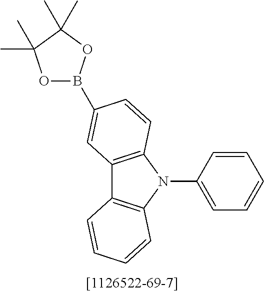 [SMILES: CC1(C)OB(c2ccc3c(c2)c2ccccc2n3-c2ccccc2)OC1(C)C] | 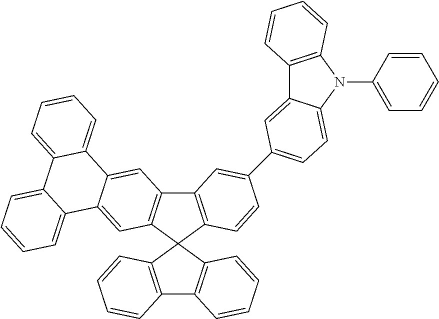 [SMILES: c1ccc(-n2c3ccccc3c3cc(-c4ccc5c(c4)-c4cc6c7ccccc7c7ccccc7c6cc4C54c5ccccc5-c5ccccc54)ccc32)cc1] | 47% |
| 24 | Inter- me- diate Ia | 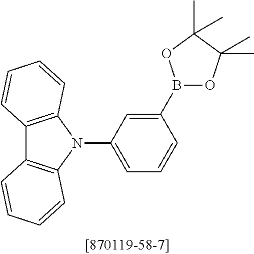 [SMILES: CC1(C)OB(c2cccc(-n3c4ccccc4c4ccccc43)c2)OC1(C)C] | 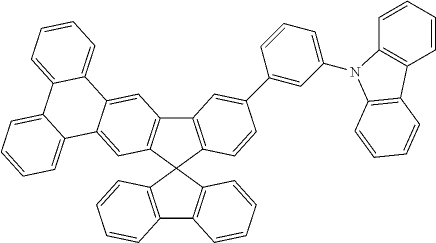 [SMILES: c1cc(-c2ccc3c(c2)-c2cc4c5ccccc5c5ccccc5c4cc2C32c3ccccc3-c3ccccc32)cc(-n2c3ccccc3c3ccccc32)c1] | 43% |
| 25 | Inter- me- diate Ia | 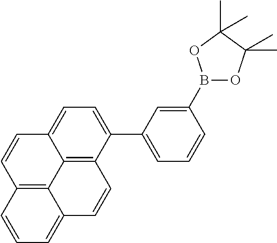 [SMILES: CC1(C)OB(c2cccc(-c3ccc4ccc5cccc6ccc3c4c56)c2)OC1(C)C] | 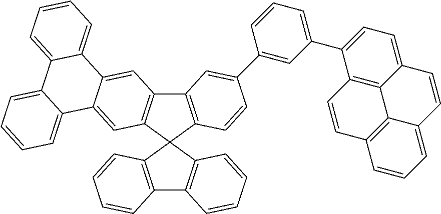 [SMILES: c1cc(-c2ccc3c(c2)-c2cc4c5ccccc5c5ccccc5c4cc2C32c3ccccc3-c3ccccc32)cc(-c2ccc3ccc4cccc5ccc2c3c45)c1] | 29% |
| 26 | Inter- me- diate Ia | 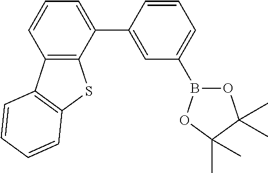 [SMILES: CC1(C)OB(c2cccc(-c3cccc4c3sc3ccccc34)c2)OC1(C)C] | 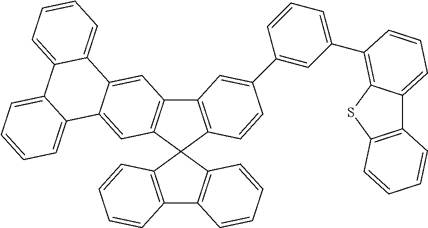 [SMILES: c1cc(-c2ccc3c(c2)-c2cc4c5ccccc5c5ccccc5c4cc2C32c3ccccc3-c3ccccc32)cc(-c2cccc3c2sc2ccccc23)c1] | 67% |
| 27 | Inter- me- diate Ib | 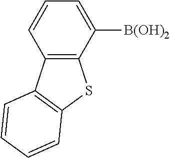 [SMILES: OB(O)c1cccc2c1sc1ccccc12] | 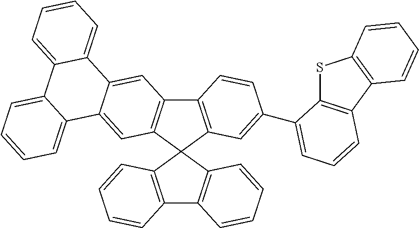 [SMILES: c1ccc2c(c1)-c1ccccc1C21c2cc(-c3cccc4c3sc3ccccc34)ccc2-c2cc3c4ccccc4c4ccccc4c3cc21] | 76% |
| 28 | Inter- me- diate Ib | 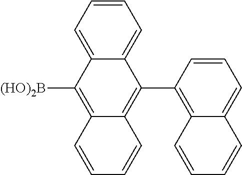 [SMILES: OB(O)c1c2ccccc2c(-c2cccc3ccccc23)c2ccccc12] | 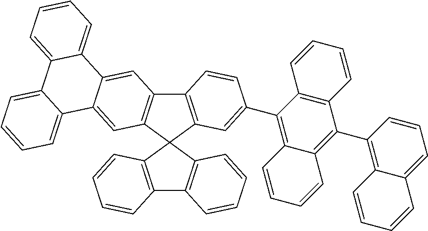 [SMILES: c1ccc2c(c1)-c1ccccc1C21c2cc(-c3c4ccccc4c(-c4cccc5ccccc45)c4ccccc34)ccc2-c2cc3c4ccccc4c4ccccc4c3cc21] | 73% |
| 29 | Inter- me- diate Ib | 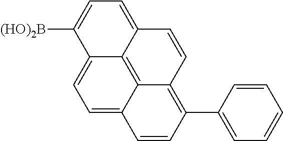 [SMILES: OB(O)c1ccc2ccc3c(-c4ccccc4)ccc4ccc1c2c43] | 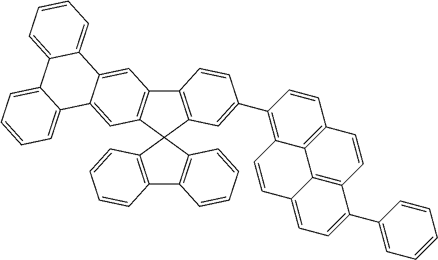 [SMILES: c1ccc(-c2ccc3ccc4c(-c5ccc6c(c5)C5(c7ccccc7-c7ccccc75)c5cc7c8ccccc8c8ccccc8c7cc5-6)ccc5ccc2c3c54)cc1] | 26% |
| 30 | Inter- me- diate Ib | 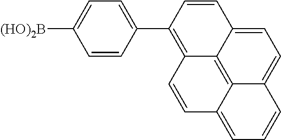 [SMILES: OB(O)c1ccc(-c2ccc3ccc4cccc5ccc2c3c45)cc1] | 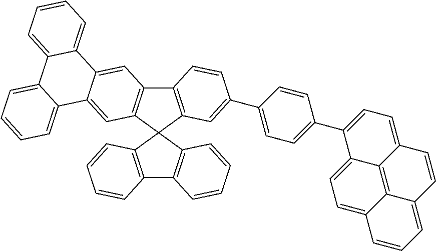 [SMILES: c1ccc2c(c1)-c1ccccc1C21c2cc(-c3ccc(-c4ccc5ccc6cccc7ccc4c5c67)cc3)ccc2-c2cc3c4ccccc4c4ccccc4c3cc21] | 37% |
| 31 | Inter- me- diate Ib | 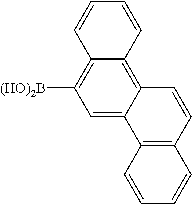 [SMILES: OB(O)c1cc2c3ccccc3ccc2c2ccccc12] | 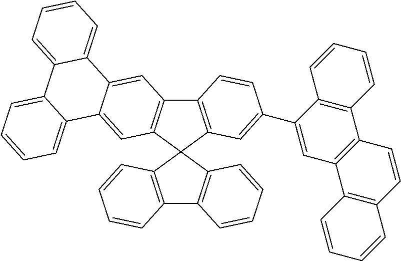 [SMILES: c1ccc2c(c1)-c1ccccc1C21c2cc(-c3cc4c5ccccc5ccc4c4ccccc34)ccc2-c2cc3c4ccccc4c4ccccc4c3cc21] | 46% |

### Example 32

### Synthesis of 4,4,5,5-tetramethyl-2-(spiro[fluorene-9, 10′-indeno[1,2-b]triphenylene]-13′-yl)-1,3,2-dioxaborolane

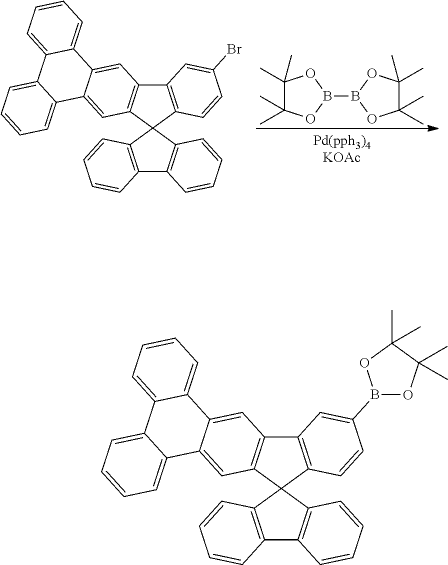

A mixture of 5.5 g (10.1 mmol) of 13′-bromospiro[fluorene-9,10′-indeno[1,2-b]triphenylene], 2.6 g (15.1 mmol) of bis(pinacolato)diboron, 0.23 (0.2 mmol) of tetrakis(triphenylphosphine)palladium, 3 g (30.3 mmol) of potassium acetate, and 60 ml of 1,4-dioxane was degassed and placed under nitrogen, and then heated at 90° C. for 4 hours. After finishing the reaction, the mixture was allowed to cool to room temperature. The organic layer was extracted with ethyl acetate and water, dried with anhydrous magnesium sulfate, the solvent was removed and the product was purified by column using a mixture of hexanes and ethyl acetate as eluent to get 4.4 g of product (yield 74%).

### Synthesis of 2-(spiro[fluorene-9,10′-indeno[1,2-b]triphenylene]-13′-yl)-1,10-phenanthroline

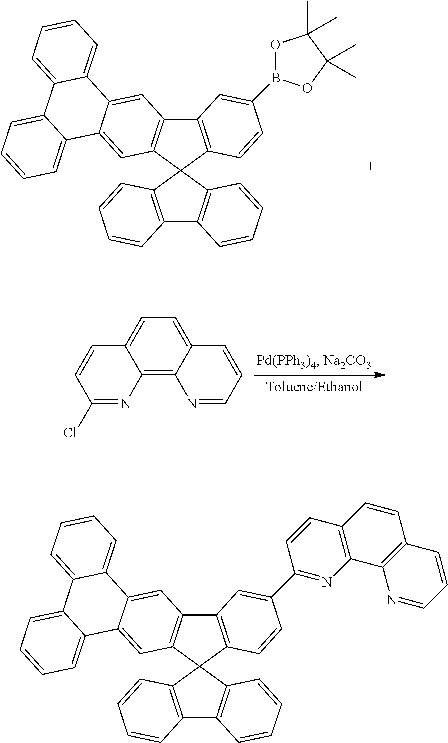 [SMILES: CC1(C)OB(c2ccc3c(c2)-c2cc4c5ccccc5c5ccccc5c4cc2C32c3ccccc3-c3ccccc32)OC1(C)C ; Clc1ccc2ccc3cccnc3c2n1 ; c1ccc2c(c1)-c1ccccc1C21c2ccc(-c3ccc4ccc5cccnc5c4n3)cc2-c2cc3c4ccccc4c4ccccc4c3cc21]

A mixture of 4.4 g (7.4 mmol) of 4,4,5,5-tetramethyl-2-(spiro[fluorene-9, 10′-indeno[1,2-b]triphenylene]-13′-yl)-1,3,2-dioxaborolane, 1.6 g (7.4 mmol) of 2-chloro-1,10-phenanthroline, 0.17 g (0.15 mmol) of tetrakis(triphenylphosphine)palladium, 10 ml of 2M Na₂CO₃, 20 ml of EtOH and 40 ml toluene was degassed and placed under nitrogen, and then heated at 90° C. overnight. After finishing the reaction, the mixture was allowed to cool to room temperature. Than 100 ml of MeOH was added, while stirring and the precipitated product was filtered off with suction. To give 4.1 g (yield 87%) of yellow product which was recrystallized from toluene. MS (m/z, FAB⁺):644.1

### Example 33

### Synthesis of 4,4,5,5-tetramethyl-2-(spiro[fluorene-9,10′-indeno[1,2-b]triphenylene]-12′-yl)-1,3,2-dioxaborolane

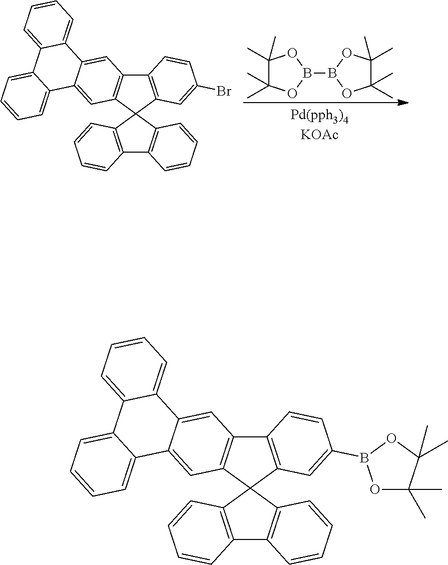

A mixture of 5.5 g (10.1 mmol) of 12′-bromospiro [fluorene-9, 10′-indeno[1,2-b]triphenylene], 2.6 g (15.1 mmol) of bis(pinacolato)diboron, 0.23 (0.2 mmol) of tetrakis(triphenylphosphine)palladium, 3 g (30.3 mmol) of potassium acetate, and 60 ml of 1,4-dioxane was degassed and placed under nitrogen, and then heated at 90° C. for 4 hours. After finishing the reaction, the mixture was allowed to cool to room temperature. The organic layer was extracted with ethyl acetate and water, dried with anhydrous magnesium sulfate, the solvent was removed and the product was purified by column using a mixture of hexanes and dichloromethane as eluent to get 4 g of product (yield 68%).

### Synthesis of 2,4-diphenyl-6-(spiro[fluorene-9, 10′-indeno[1,2-b]triphenylene]-12′-yl)-1,3,5-triazine

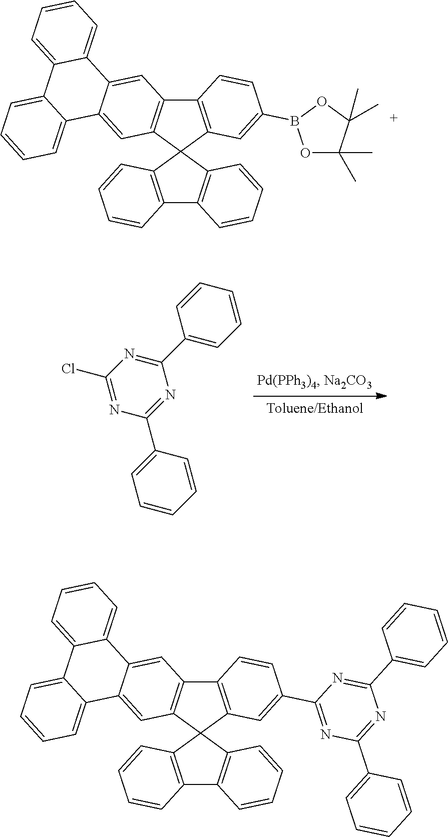

A mixture of 4 g (6.8 mmol) of 4,4,5,5-tetramethyl-2-(spiro[fluorene-9, 10′-indeno[1,2-b]triphenylene]-12′-yl)-1,3,2-dioxaborolane, 1.6 g (10 mmol) of 2-chloro-4,6-diphenyl-1,3,5-triazine, 0.17 g (0.15 mmol) of tetrakis(triphenylphosphine)palladium, 10 ml of 2M Na₂CO₃, 20 ml of EtOH and 40 ml toluene was degassed and placed under nitrogen, and then heated at 90° C. overnight. After finishing the reaction, the mixture was allowed to cool to room temperature. The organic layer was extracted with ethyl acetate and water, dried with anhydrous magnesium sulfate, the solvent was removed and the product was purified by column using a mixture of hexanes and dichloromethane as eluent to get 3.7 g of product (yield 78%). MS (m/z, FAB⁺):697.8

### Example 34

### Synthesis of 2-(phenanthro[9,10-b]tetraphenylen-11-yl)-4,6-diphenylpyrimidine

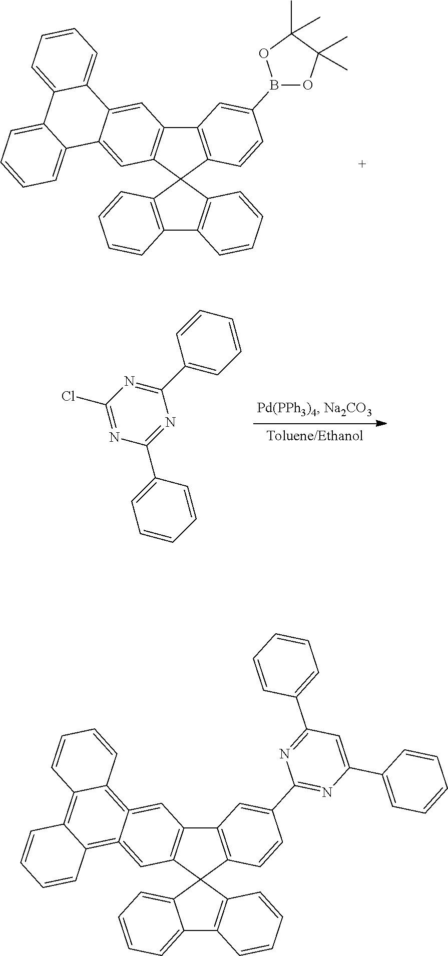

2-chloro-4,6-diphenyl-1,3,5-triazine instead of 2-chloro-1,10-phenanthroline, except for using the same method as in synthesis example 32, the desired compound of example 34 (8.1 g, yield=47%) was obtained. MS (m/z, FAB⁺):684.8

### Example 35

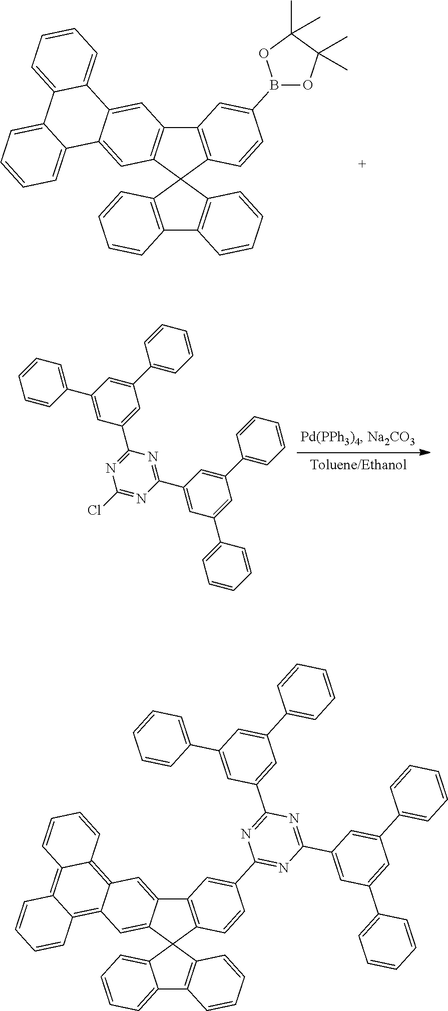 [SMILES: CC1(C)OB(c2ccc3c(c2)-c2cc4c5ccccc5c5ccccc5c4cc2C32c3ccccc3-c3ccccc32)OC1(C)C ; "+" ; Clc1nc(-c2cc(-c3ccccc3)cc(-c3ccccc3)c2)nc(-c2cc(-c3ccccc3)cc(-c3ccccc3)c2)n1 ; c1ccc(-c2cc(-c3ccccc3)cc(-c3nc(-c4cc(-c5ccccc5)cc(-c5ccccc5)c4)nc(-c4ccc5c(c4)-c4cc6c7ccccc7c7ccccc7c6cc4C54c5ccccc5-c5ccccc54)n3)c2)cc1]

2-chloro-(4,6-bis[3,1,5,1]terphen-1-yl)-1,3,5-triazine instead of 2-chloro-1,10-phenanthroline, except for using the same method as in synthesis example 32, the desired compound of example 35 (3.6 g, yield=51%) was obtained. MS (m/z, FAB⁺):989.3

### Example 36

### Synthesis of 2-phenyl-9-(spiro[fluorene-9,10′-indeno[2,1-b]triphenylene]-13′-yl)-1,10-phenanthroline

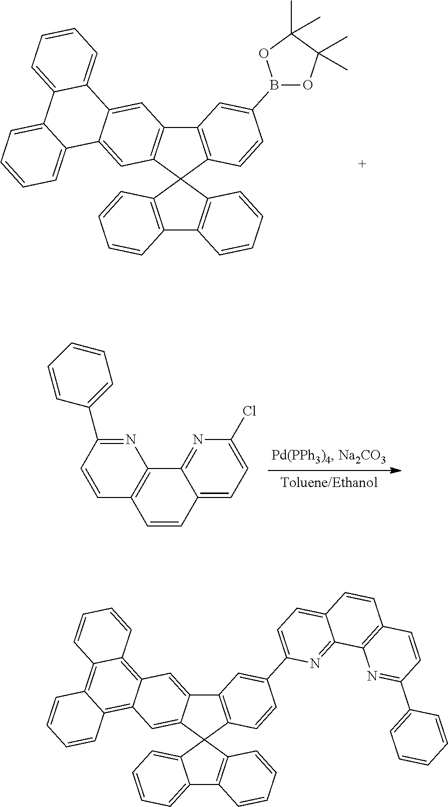 [SMILES: CC1(C)OB(c2ccc3c(c2)-c2cc4c5ccccc5c5ccccc5c4cc2C32c3ccccc3-c3ccccc32)OC1(C)C ; "+" ; Clc1ccc2ccc3ccc(-c4ccccc4)nc3c2n1 ; c1ccc(-c2ccc3ccc4ccc(-c5ccc6c(c5)-c5cc7c8ccccc8c8ccccc8c7cc5C65c6ccccc6-c6ccccc65)nc4c3n2)cc1]

2-chloro-9-phenyl-1,10-phenanthroline instead of 2-chloro-1,10-phenanthroline, except for using the same method as in synthesis example 32, the desired compound of example 36 (9.6 g, yield=62%) was obtained. MS (m/z, FAB⁺):708.4

### Example 37

### Synthesis of 9,9-dimethyl-10-phenyl-2-(spiro[fluorene-9,10′-indeno[2,1-b]triphenylene]-13′-yl)-9,10-dihydroacridine

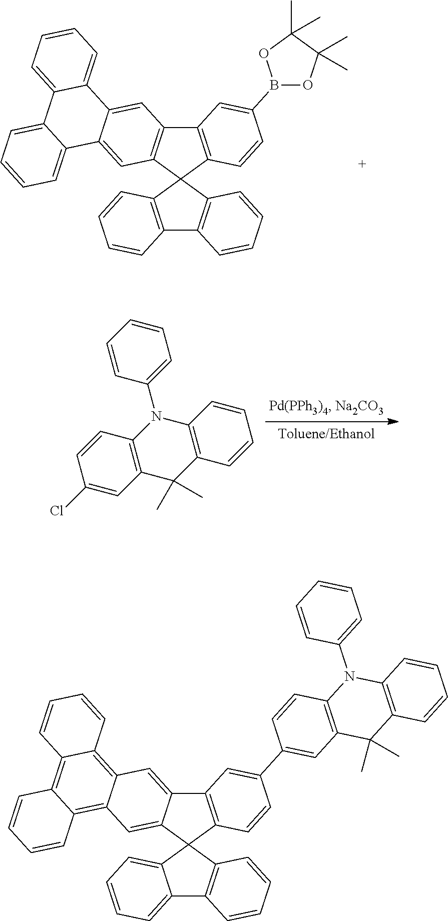 [SMILES: CC1(C)OB(c2ccc3c(c2)-c2cc4c5ccccc5c5ccccc5c4cc2C32c3ccccc3-c3ccccc32)OC1(C)C ; "+" ; CC1(C)c2ccccc2N(c2ccccc2)c2ccc(Cl)cc21 ; CC1(C)c2ccccc2N(c2ccccc2)c2ccc(-c3ccc4c(c3)-c3cc5c6ccccc6c6ccccc6c5cc3C43c4ccccc4-c4ccccc43)cc21]

2-chloro-9,9-dimethyl-10-phenyl-9,10-dihydroacridine instead of 2-chloro-1,10-phenanthroline, except for using the same method as in synthesis example 32, the desired compound of example 37 (4.9 g, yield=52%) was obtained. MS (m/z, FAB⁺):749.3

### Example 38

### Synthesis of 9-(spiro[fluorene-9,10′-indeno[1,2-b]triphenylene]-13′-yl)-9H-carbazole

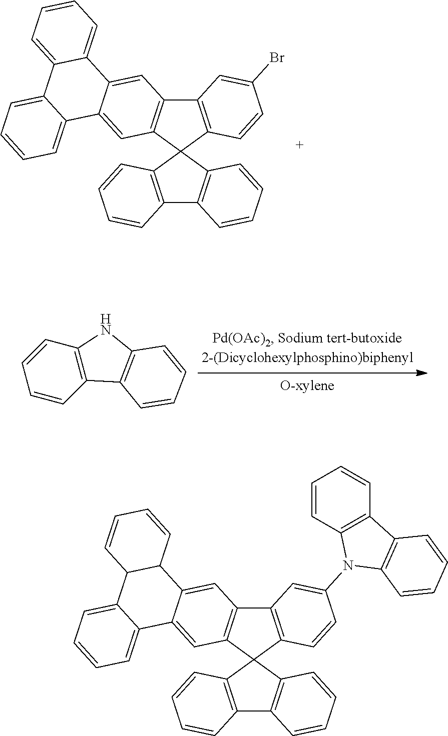 [SMILES: Brc1ccc2c(c1)-c1cc3c4ccccc4c4ccccc4c3cc1C21c2ccccc2-c2ccccc21 ; "+" ; c1ccc2c(c1)[nH]c1ccccc12 ; C1=CC2c3ccccc3-c3cc4c(cc3C2C=C1)-c1cc(-n2c3ccccc3c3ccccc32)ccc1C41c2ccccc2-c2ccccc21]

A mixture of 5.5 g (10.1 mmol) 13′-bromospiro[fluorene-9,10′-indeno[1,2-b]triphenylene], 2.5 g (15.1 mmol) of 9H-carbazole, 0.05 g (0.2 mmol) of palladium(II)acetate, 0.15 g (0.4 mmol) of 2-(dicyclohexylphosphino)biphenyl, 2 g (20 mmol) of sodium tert-butoxide and 100 ml of o-xylene was refluxed under nitrogen overnight. After finishing the reaction, the solution was filtered at 100° C., To receive the filtrate, and the filtrate was added to 1 L MeOH, while stirring and the precipitated product was filtered off with suction. To give 3.4 g (yield 53%) of yellow product which was recrystallized from toluene. MS (m/z, FAB⁺):632.1

### Example 39

### Synthesis of 9-(spiro[fluorene-9,10′-indeno[1,2-b]triphenylene]-12′-yl)-9H-carbazole

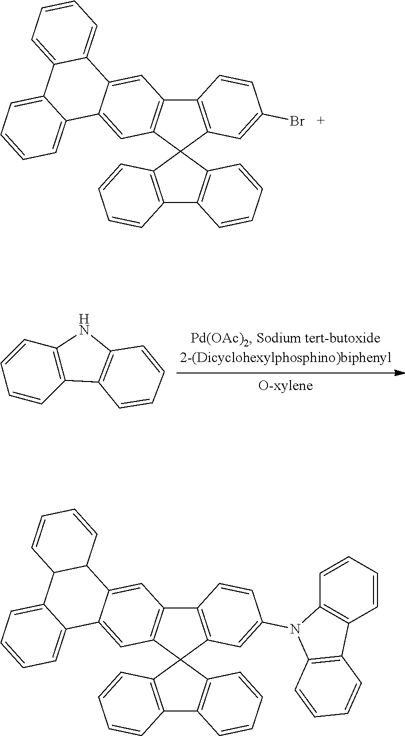

A mixture of 5.5 g (10.1 mmol) 12′-bromospiro[fluorene-9,10′-indeno[1,2-b]triphenylene], 2.5 g (15.1 mmol) of 9H-carbazole, 0.05 g (0.2 mmol) of palladium(II)acetate, 0.15 g (0.4 mmol) of 2-(dicyclohexylphosphino)biphenyl, 2 g (20 mmol) of sodium tert-butoxide and 100 ml of o-xylene was refluxed under nitrogen overnight. After finishing the reaction, the solution was filtered at 100° C., to receive the filtrate, and the filtrate was added to 1 L MeOH, while stirring and the precipitated product was filtered off with suction. To give 2.9 g (yield 45%) of yellow product which was recrystallized from toluene. MS (m/z, FAB⁺):632.1

### Example 40

### Synthesis of 9-phenyl-9′-(spiro[fluorene-9,10′-indeno[1,2-b]triphenylene]-13′-yl)-9H,9′H-3,3′-bicarbazole

A mixture of 5.5 g (10.1 mmol) 13′-bromospiro[fluorene-9,10′-indeno[1,2-b]triphenylene], 6.2 g (15.1 mmol) of 9-phenyl-9H,9′H-3,3′-bicarbazole, 0.05 g (0.2 mmol) of palladium(II)acetate, 0.15 g (0.4 mmol) of 2-(dicyclohexylphosphino)biphenyl, 2 g (20 mmol) of sodium tert-butoxide and 100 ml of o-xylene was refluxed under nitrogen overnight. After finishing the reaction, the solution was filtered at 100° C., to receive the filtrate, and the filtrate was added to 1 L MeOH, while stirring and the precipitated product was filtered off with suction. To give 4.1 g (yield 47%) of yellow product which was recrystallized from toluene. MS (m/z, FAB⁺):872.9

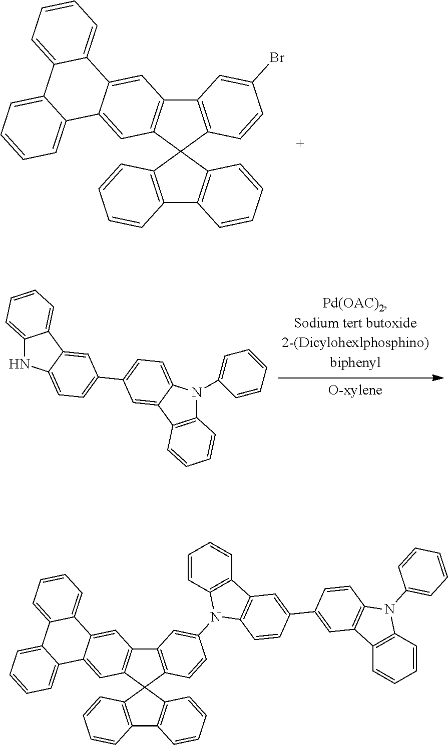 [SMILES: Brc1ccc2c(c1)-c1cc3c4ccccc4c4ccccc4c3cc1C21c2ccccc2-c2ccccc21 ; "+" ; c1ccc(-n2c3ccccc3c3cc(-c4ccc5[nH]c6ccccc6c5c4)ccc32)cc1 ; c1ccc(-n2c3ccccc3c3cc(-c4ccc5c(c4)c4ccccc4n5-c4ccc5c(c4)-c4cc6c7ccccc7c7ccccc7c6cc4C54c5ccccc5-c5ccccc54)ccc32)cc1]

### Example 41

### Synthesis of 9-(spiro[fluorene-9,10′-indeno[1,2-b]triphenylene]-13′-yl)-9H-3,9′-bicarbazole

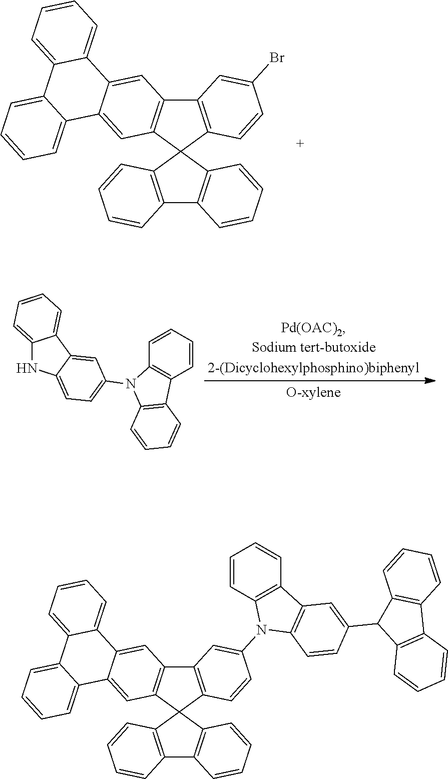 [SMILES: Brc1ccc2c(c1)-c1cc3c4ccccc4c4ccccc4c3cc1C21c2ccccc2-c2ccccc21 ; "+" ; c1ccc2c(c1)[nH]c1ccc(-n3c4ccccc4c4ccccc43)cc12 ; c1ccc2c(c1)-c1ccccc1C2c1ccc2c(c1)c1ccccc1n2-c1ccc2c(c1)-c1cc3c4ccccc4c4ccccc4c3cc1C21c2ccccc2-c2ccccc21]

A mixture of 5.5 g (10.1 mmol) 13′-bromospiro[fluorene-9,10′-indeno[1,2-b]triphenylene], 5 g (15.1 mmol) of 9H-3,9′-bicarbazole, 0.05 g (0.2 mmol) of palladium(II)acetate, 0.15 g (0.4 mmol) of 2-(dicyclohexyl-phosphino)biphenyl, 2 g (20 mmol) of sodium tert-butoxide and 100 ml of o-xylene was refluxed under nitrogen overnight. After finishing the reaction, the solution was filtered at 100° C., to receive the filtrate, and the filtrate was added to 1 L MeOH, while stirring and the precipitated product was filtered off with suction. To give 4.1 g (yield 51%) of yellow product which was recrystallized from toluene. MS (m/z, FAB⁺):796.5

### Example 42

### Synthesis of 10-(spiro[fluorene-9,10′-indeno[1,2-b]triphenylene]-13′-yl)-1 OH-phenothiazine

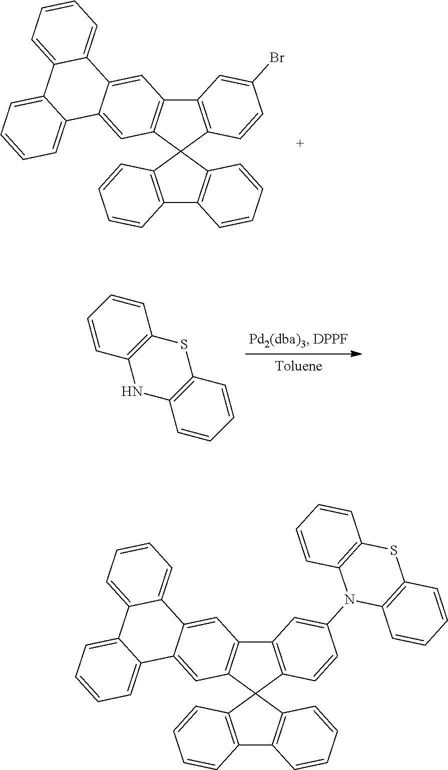 [SMILES: Brc1ccc2c(c1)-c1cc3c4ccccc4c4ccccc4c3cc1C21c2ccccc2-c2ccccc21 ; "+" ; c1ccc2c(c1)Nc1ccccc1S2 ; c1ccc2c(c1)Sc1ccccc1N2c1ccc2c(c1)-c1cc3c4ccccc4c4ccccc4c3cc1C21c2ccccc2-c2ccccc21]

To a solution of tris(dibenzylideneacetone)dipalladium(0.30 g, 0.33 mmol) and bis(diphenylphosphino)ferrocene (0.22 g, 0.40 mmol) in 160 ml dry toluene under nitrogen atmosphere was added 8.2 g of 13′-bromospiro[fluorene-9,10′-indeno[1,2-b]triphenylene] at room temperature, and the resultant mixture was stirred for 10 min., 4 g of sodium tert-butoxide and 3 g of 10H-phenothiazine (15.1 mmol) were added to this solution and stirred at 110° C. overnight under nitrogen. The reaction mixture was poured into 300 ml of water, the organic layer was extracted with ethyl acetate and water, dried with anhydrous magnesium sulfate, the solvent was removed and the product was purified by column using a mixture of hexanes and dichloromethane as eluent to get 6.3 g of product (yield 63%). MS (m/z, FAB⁺):663.9

### Example 43

### Synthesis of 9,9-dimethyl-10-(spiro[fluorene-9,10′-indeno[1,2-b]triphenylene]-13′-yl)-9,10-dihydroacridine

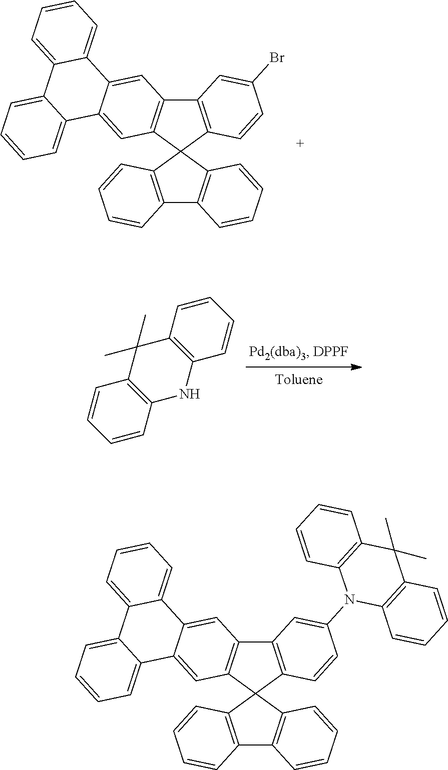 [SMILES: Brc1ccc2c(c1)-c1cc3c4ccccc4c4ccccc4c3cc1C21c2ccccc2-c2ccccc21 ; "+" ; CC1(C)c2ccccc2Nc2ccccc21 ; CC1(C)c2ccccc2N(c2ccc3c(c2)-c2cc4c5ccccc5c5ccccc5c4cc2C32c3ccccc3-c3ccccc32)c2ccccc21]

To a solution of tris(dibenzylideneacetone)dipalladium(0.30 g, 0.33 mmol) and bis(diphenylphosphino)ferrocene (0.22 g, 0.40 Mmol) in 160 ml dry toluene under nitrogen atmosphere was added 8.2 g of 13′-bromospiro[fluorene-9,10′-indeno[1,2-b]triphenylene] at room tempera ture, and the resultant mixture was stirred for 10 min., 4 g of sodium tert butoxide and 3.2 g of 9,9-dimethyl-9,10-dihydroacridine (15. mmol) were added to this solution and stirred at 110° C. overnight under nitrogen. The reaction mixture was poured into 300 ml of water, the organic layer was extracted with ethyl acetate and water, dried with anhydrous magnesium sulfate, the solvent was removed and the product was purified by column using a mixture of hexanes 25.1 g (110 mmol) of 5-methoxybiphenyl-2-ylboronic acid, 0.24 g (0.2 mmol) of tetrakis(triphenylphosphine)palladium, 100 ml of 2M Na₂CO₃, 125 ml of EtOH and 250 ml toluene was degassed and placed under nitrogen, and then heated at 110° C. for 16 hours. After finishing the reaction, the mixture was allowed to cool to room temperature. The reaction mixture was extracted with ethyl acetate and water, dried with anhydrous magnesium sulfate, the solvent was evaporated in vacuum. The residue was purified by column using a mixture of hexanes and dichloromethane as eluent to get 36.4 g of product (yield 73%)

### Example 44

### Synthesis of 2-(3-(4,4,5,5-tetramethyl-1,3,2-dioxaborolan-2-yl)phenyl)-1,10-phenanthroline

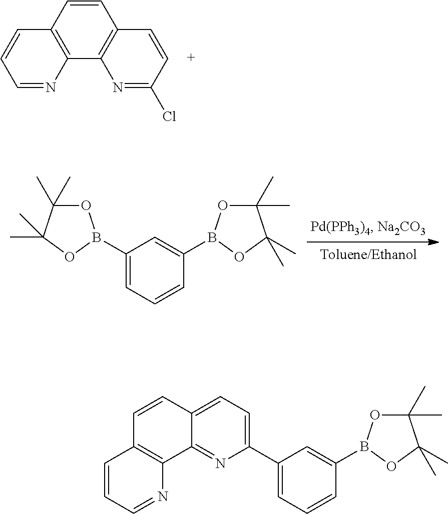

A mixture of 16.5 g (50 mmol) of 1,3-bis(4,4,5,5-tetramethyl-1,3,2-dioxaborolan-2-yl)benzene, 10.7 g (50 mmol) of 2-chloro-1,10-phenanthroline, 1.15 g (1 mmol) of tetrakis(triphenylphosphine)palladium, 37 ml of 2M Na₂CO₃, 50 ml of EtOH and 100 ml toluene was degassed and placed under nitrogen, and then heated at 75° C. overnight. After finishing the reaction, the mixture was allowed to cool to room temperature. The reaction mixture was extracted with ethyl acetate and water, dried with anhydrous magnesium sulfate, the solvent was evaporated in vacuum. The residue was purified by column using a mixture of dichloromethane and 5% MeOH as eluent to get 7.8 g of product (yield 41%)

### Synthesis of 2-(3-(spiro[fluorene-9,10′-indeno[1,2-b]triphenylene]-6′-yl)phenyl)-1,10-phenanthroline

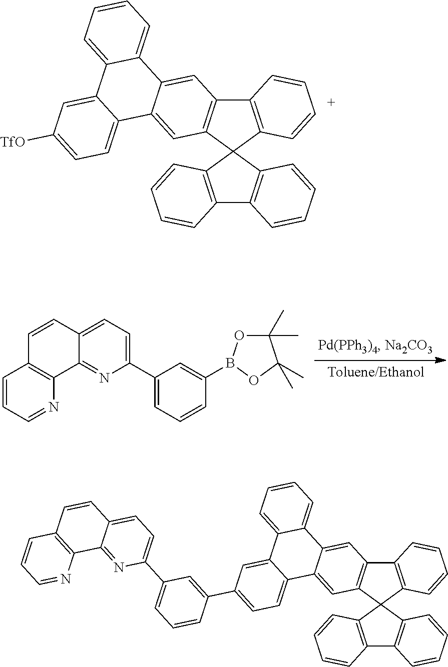 [SMILES: O=S(=O)(Oc1ccc2c(c1)c1ccccc1c1cc3c(cc21)C1(c2ccccc2-c2ccccc21)c1ccccc1-3)C(F)(F)F ; CC1(C)OB(c2cccc(-c3ccc4ccc5cccnc5c4n3)c2)OC1(C)C ; c1cc(-c2ccc3c(c2)c2ccccc2c2cc4c(cc32)C2(c3ccccc3-c3ccccc32)c2ccccc2-4)cc(-c2ccc3ccc4cccnc4c3n2)c1]

A mixture of 5.7 g (15 mmol) of 2-(3-(4,4,5,5-tetramethyl-1,3,2-dioxaborolan-2-yl)phenyl)-1,10-phenanthroline, 9.2 g (15 mmol) of spiro[fluorene-9,10′-indeno[2,1-b]triphenylene]-6′-yltrifluoromethanesulfonate, 0.17 g (0.15 mmol) of tetrakis(triphenylphosphine)palladium, 10 ml of 2M Na₂CO₃, 20 ml of EtOH and 40 ml toluene was degassed and placed under nitrogen, and then heated at 90° C. overnight. After finishing the reaction, the mixture was allowed to cool to room temperature. Than 100 ml of MeOH was added, while stirring and the precipitated product was filtered off with suction. To give 8.2 g (yield 76%) of yellow product which was recrystallized from toluene. MS (m/z, FAB⁺):720.8

### Example 45

### Synthesis of 4-(spiro[fluorene-9,10′-indeno[1,2-b]triphenylene]-6′-yl)dibenzo[b,d]thiophene

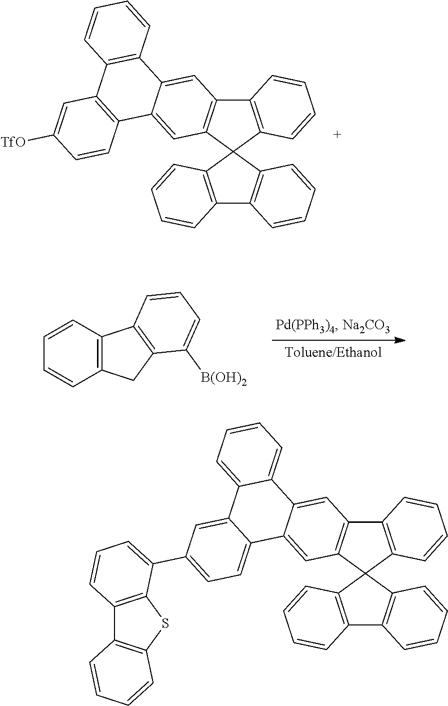 [SMILES: O=S(=O)(Oc1ccc2c(c1)c1ccccc1c1cc3c(cc21)C1(c2ccccc2-c2ccccc21)c1ccccc1-3)C(F)(F)F ; OB(O)c1cccc2c1Cc1ccccc1-2 ; c1ccc2c(c1)-c1ccccc1C21c2ccccc2-c2cc3c4ccccc4c4cc(-c5cccc6c5sc5ccccc56)ccc4c3cc21]

A mixture of 3.4 g (15 mmol) of dibenzo[b,d]thiophen-4-ylboronic acid, 9.2 g (15 mmol) of spiro[fluorene-9,10′-indeno[2,1-b]triphenylene]-6′-yl trifluoromethanesulfonate, 0.17 g (0.15 mmol) of tetrakis(triphenylphosphine) palladium, 10 ml of 2M Na₂CO₃, 20 ml of EtOH and 40 ml toluene was degassed and placed under nitrogen, and then heated at 90° C. overnight. After finishing the reaction, the mixture was allowed to cool to room temperature. Than 100 ml of MeOH was added, while stirring and the precipitated product was filtered off with suction. To give 4.5 g (yield 47%) of yellow product which was recrystallized from toluene. MS (m/z, FAB⁺):648.6

### Example 46

### Synthesis of 2-chloro-4,6-diphenylpyrimidine

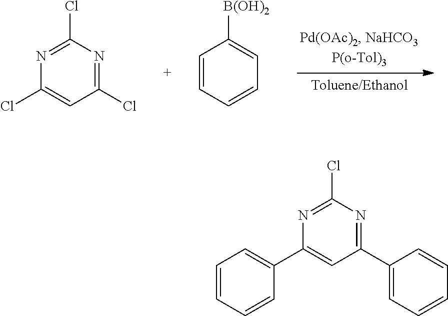 [SMILES: Clc1cc(Cl)nc(Cl)n1 ; OB(O)c1ccccc1 ; Clc1nc(-c2ccccc2)cc(-c2ccccc2)n1]

75 g (410 mmol) of 1,3,5-trichloropyrimidine, 100 g (820 mmol) of phenylboronic acid and 615 ml of 2M NaHCO₃ solution are suspended in 1200 ml of ethylene glycol dimethyl ether. 1.9 g (8.4 mmol) of Pd(OAc)₂ and 5.2 g (17 mmol) of P (o-Tol)₃ was added to the suspension, and the reaction mixture was heated under reflux overnight. After finishing the reaction, the mixture was allowed to cool to room temperature. The reaction mixture was extracted with ethyl acetate and water, dried with anhydrous magnesium sulfate and evaporated in a rotary evaporator. The residue remaining is recrystallized from toluene. The yield is 46 g (0.15 mmol, 42%).

### Synthesis of 4,6-diphenyl-2-(3-(4,4,5,5-tetramethyl-1,3,2-dioxaborolan-2-yl)phenyl)pyrimidine

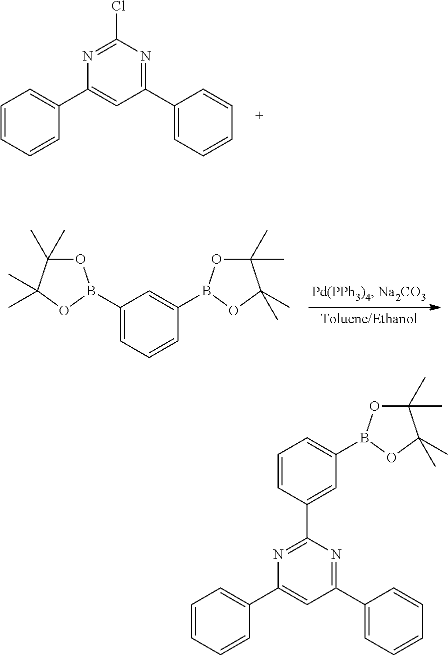

A mixture of 16.5 g (50 mmol) of 1,3-bis(4,4,5,5-tetramethyl-1,3,2-dioxaborolan-2-yl)benzene, 13.3 g (50 mmol) of 2-chloro-4,6-diphenylpyrimidine, 1.15 g (1 mmol) of tetrakis(triphenylphosphine)palladium, 37 ml of 2M Na₂CO₃, 50 ml of EtOH and 100 ml toluene was degassed and placed under nitrogen, and then heated at 75° C. overnight. After finishing the reaction, the mixture was allowed to cool to room temperature. The reaction mixture was extracted with ethyl acetate and water, dried with anhydrous magnesium sulfate, the solvent was evaporated in vacuum. The residue was purified by column using a mixture of hexane and 30% ethyl acetate as eluent to get 8 g of product (yield 37%)

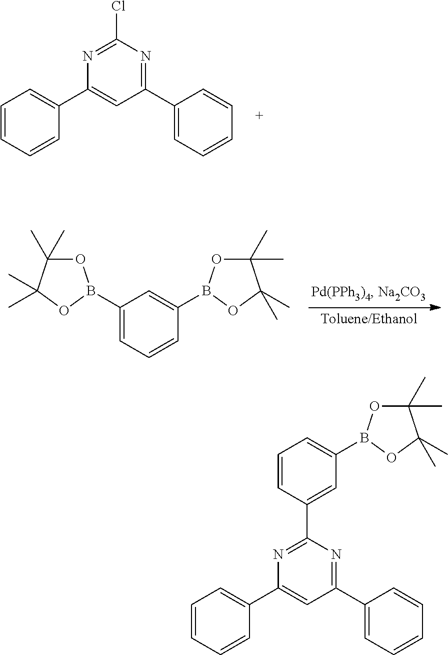

### Synthesis of 4,6-diphenyl-2-(3-(spiro[fluorene-9,10′-indeno[1,2-b]triphenylene]-6′-yl)phenyl)pyrimidine

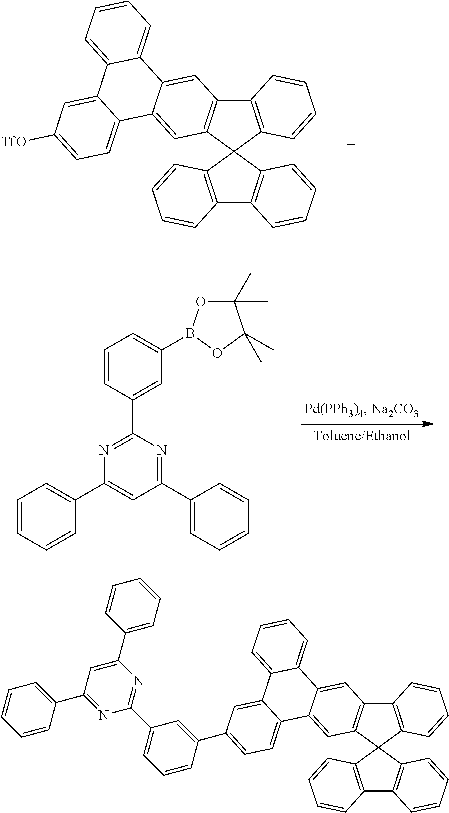 [SMILES: O=S(=O)(Oc1ccc2c(c1)c1ccccc1c1cc3c(cc21)C1(c2ccccc2-c2ccccc21)c1ccccc1-3)C(F)(F)F ; CC1(C)OB(c2cccc(-c3nc(-c4ccccc4)cc(-c4ccccc4)n3)c2)OC1(C)C ; c1ccc(-c2cc(-c3ccccc3)nc(-c3cccc(-c4ccc5c(c4)c4ccccc4c4cc6c(cc54)C4(c5ccccc5-c5ccccc54)c4ccccc4-6)c3)n2)cc1]

A mixture of 6.5 g (15 mmol) of 4,6-diphenyl-2-(3-(4,4,5,5-tetramethyl-1,3,2-dioxaborolan-2-yl)phenyl)pyramidine, 9.2 g (15 mmol) of spiro[fluorene-9,10′-indeno[2,1-b]triphenylene]-6′-yltrifluoromethanesulfonat e, 0.17 g (0.15 mmol) of tetrakis(triphenylphosphine)palladium, 10 ml of 2M Na₂CO₃, 20 ml of EtOH and 40 ml toluene was degassed and placed under nitrogen, and then heated at 90° C. overnight. After finishing the reaction, the mixture was allowed to cool to room temperature. Than 100 ml of MeOH was added, while stirring and the precipitated product was filtered off with suction. To give 7.7 g (yield 67%) of yellow product which was recrystallized from toluene. MS (m/z, FAB⁺):772.4

### Example 47˜55

The following compounds are synthesized analogously

| | Inter- | | | |
|---|---|---|---|---|
| | me- | | | |
| Ex. | diate I | Intermediate II | Product | Yield |
| 47 | Inter- me- diate Ic | 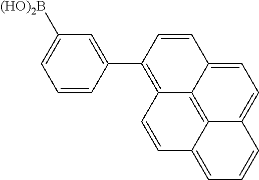 [SMILES: OB(O)c1cccc(-c2ccc3ccc4cccc5ccc2c3c45)c1] | 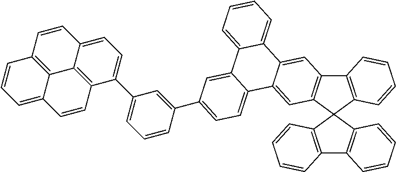 [SMILES: c1cc(-c2ccc3c(c2)c2ccccc2c2cc4c(cc32)C2(c3ccccc3-c3ccccc32)c2ccccc2-4)cc(-c2ccc3ccc4cccc5ccc2c3c45)c1] | 36% |
| 48 | Inter- me- diate Ic | 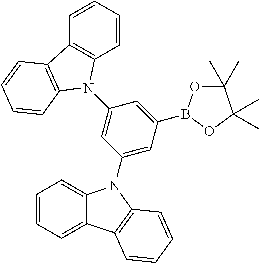 [SMILES: CC1(C)OB(c2cc(-n3c4ccccc4c4ccccc43)cc(-n3c4ccccc4c4ccccc43)c2)OC1(C)C] | 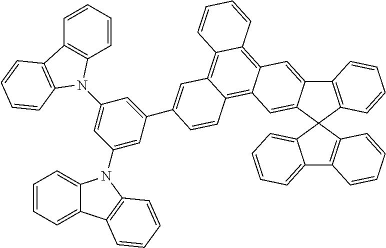 [SMILES: c1ccc2c(c1)-c1ccccc1C21c2ccccc2-c2cc3c4ccccc4c4cc(-c5cc(-n6c7ccccc7c7ccccc76)cc(-n6c7ccccc7c7ccccc76)c5)ccc4c3cc21] | 27% |
| 49 | Inter- me- diate Ic | 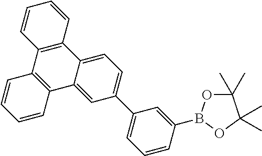 [SMILES: CC1(C)OB(c2cccc(-c3ccc4c5ccccc5c5ccccc5c4c3)c2)OC1(C)C] | 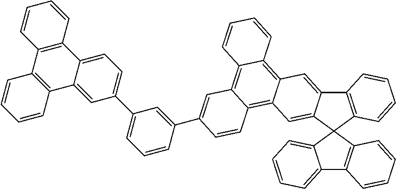 [SMILES: c1cc(-c2ccc3c4ccccc4c4ccccc4c3c2)cc(-c2ccc3c(c2)c2ccccc2c2cc4c(cc32)C2(c3ccccc3-c3ccccc32)c2ccccc2-4)c1] | 31% |
| 50 | Inter- me- diate Ic | 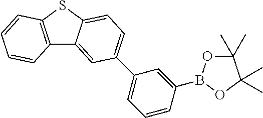 [SMILES: CC1(C)OB(c2cccc(-c3ccc4sc5ccccc5c4c3)c2)OC1(C)C] | 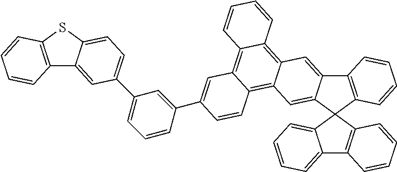 [SMILES: c1cc(-c2ccc3sc4ccccc4c3c2)cc(-c2ccc3c(c2)c2ccccc2c2cc4c(cc32)C2(c3ccccc3-c3ccccc32)c2ccccc2-4)c1] | 41% |
| 51 | Inter- me- diate Ic | 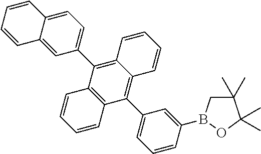 [SMILES: CC1(C)CB(c2cccc(-c3c4ccccc4c(-c4ccc5ccccc5c4)c4ccccc34)c2)OC1(C)C] | 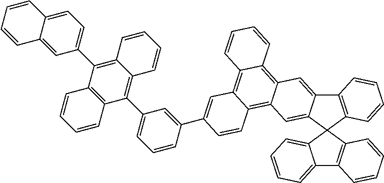 [SMILES: c1cc(-c2ccc3c(c2)c2ccccc2c2cc4c(cc32)C2(c3ccccc3-c3ccccc32)c2ccccc2-4)cc(-c2c3ccccc3c(-c3ccc4ccccc4c3)c3ccccc23)c1] | 46% |
| 52 | Inter- me- diate Ic | 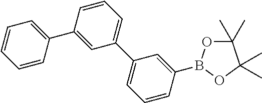 [SMILES: CC1(C)OB(c2cccc(-c3cccc(-c4ccccc4)c3)c2)OC1(C)C] | 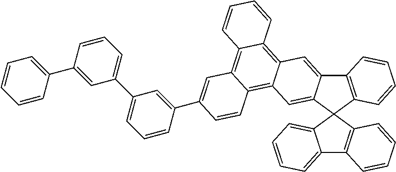 [SMILES: c1ccc(-c2cccc(-c3cccc(-c4ccc5c(c4)c4ccccc4c4cc6c(cc54)C4(c5ccccc5-c5ccccc54)c4ccccc4-6)c3)c2)cc1] | 35% |
| 53 | Inter- me- diate Ic | 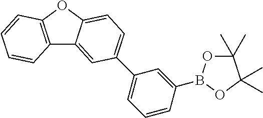 [SMILES: CC1(C)OB(c2cccc(-c3ccc4oc5ccccc5c4c3)c2)OC1(C)C] | 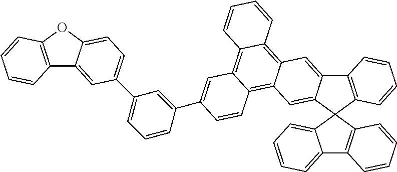 [SMILES: c1cc(-c2ccc3oc4ccccc4c3c2)cc(-c2ccc3c(c2)c2ccccc2c2cc4c(cc32)C2(c3ccccc3-c3ccccc32)c2ccccc2-4)c1] | 39% |
| 54 | Inter- me- diate Ic | 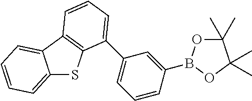 [SMILES: CC1(C)OB(c2cccc(-c3cccc4c3sc3ccccc34)c2)OC1(C)C] | 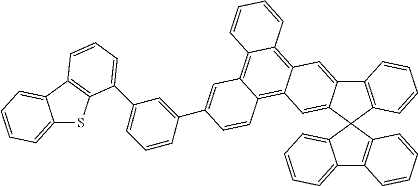 [SMILES: c1cc(-c2ccc3c(c2)c2ccccc2c2cc4c(cc32)C2(c3ccccc3-c3ccccc32)c2ccccc2-4)cc(-c2cccc3c2sc2ccccc23)c1] | 47% |
| 55 | Inter- me- diate Ic | 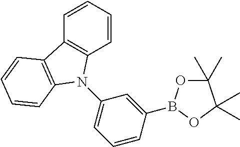 [SMILES: CC1(C)OB(c2cccc(-n3c4ccccc4c4ccccc43)c2)OC1(C)C] | 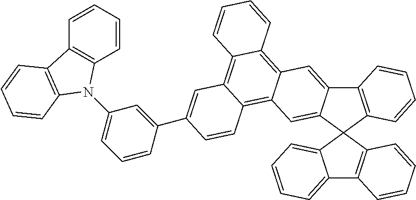 [SMILES: c1cc(-c2ccc3c(c2)c2ccccc2c2cc4c(cc32)C2(c3ccccc3-c3ccccc32)c2ccccc2-4)cc(-n2c3ccccc3c3ccccc32)c1] | 39% |

### Example 56

### Synthesis of 9-phenyl-9H,9′H-3,3′-bicarbazole

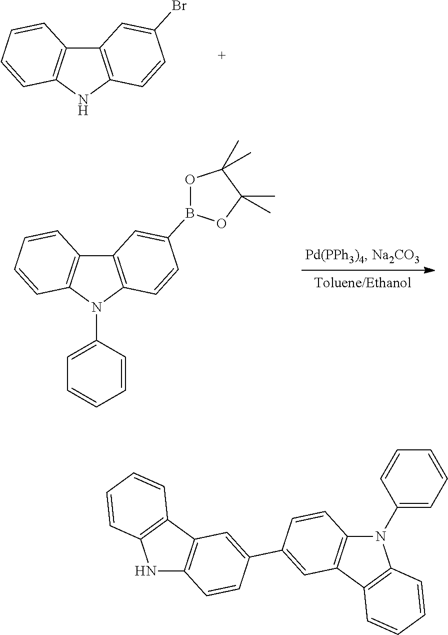 [SMILES: Brc1ccc2[nH]c3ccccc3c2c1 ; CC1(C)OB(c2ccc3c(c2)c2ccccc2n3-c2ccccc2)OC1(C)C ; c1ccc(-n2c3ccccc3c3cc(-c4ccc5[nH]c6ccccc6c5c4)ccc32)cc1]

A mixture of 13.5 g (55 mmol) of 3-bromo-9H-carbazole, 22 g (60 mmol) of 9-phenyl-3-(4,4,5,5-tetramethyl-1,3,2-dioxaborolan-2-yl)-9H-carbazole, 1.7 g (1.5 mmol) of tetrakis(triphenylphosphine)palladium, 55 ml of 2M Na₂CO₃, 70 ml of EtOH and 200 ml toluene was degassed and placed under nitrogen, and then heated at 90° C. overnight. After finishing the reaction, the mixture was allowed to cool to room temperature. The organic layer was extracted with ethyl acetate and water, dried with anhydrous magnesium sulfate, the solvent was removed and the product was purified by column using a mixture of hexane and dichloromethane as eluent to get 7.6 of product (yield 34%).

### Synthesis of 9-phenyl-9′-(spiro[fluorene-9,10′-indeno[2,1-b]triphenylene]-6′-yl)-9H,9′H-3,3′-bicarbazole

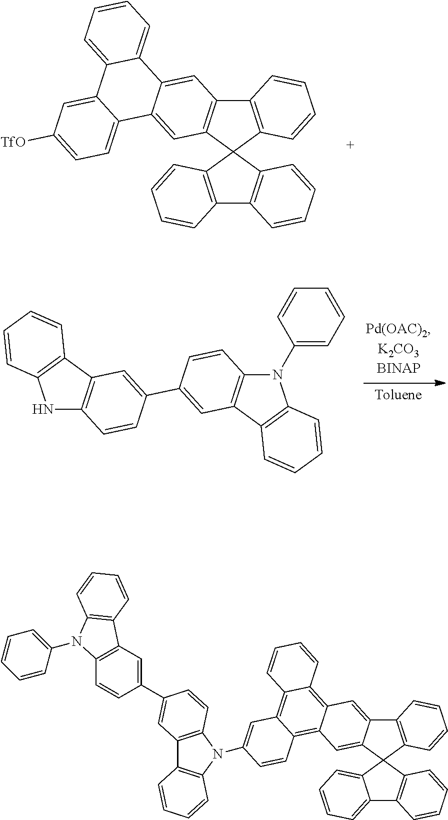 [SMILES: O=S(=O)(Oc1ccc2c(c1)c1ccccc1c1cc3c(cc21)C1(c2ccccc2-c2ccccc21)c1ccccc1-3)C(F)(F)F ; c1ccc(-n2c3ccccc3c3cc(-c4ccc5[nH]c6ccccc6c5c4)ccc32)cc1 ; c1ccc(-n2c3ccccc3c3cc(-c4ccc5c(c4)c4ccccc4n5-c4ccc5c(c4)c4ccccc4c4cc6c(cc54)C4(c5ccccc5-c5ccccc54)c4ccccc4-6)ccc32)cc1]

A mixture of 4.1 g (10 mmol) of 9-phenyl-9H,9′H-3,3′-bicarbazole, 6.1 g (10 mmol) of spiro[fluorene-9,10′-indeno[2,1-b]triphenylene]-6′-yl trifluoromethanesulfonate, 0.09 g (0.4 mmol) of palladium(II)acetate, 0.48 g of BINAP, 3.5 g of potassium carbonate and 150 ml toluene was degassed and placed under nitrogen, and then heated at 110° C. overnight. After finishing the reaction, the mixture was allowed to cool to room temperature. Than 500 ml of MeOH was added, while stirring and the precipitated product was filtered off with suction. To give 7.7 g (yield 67%) of yellow product which was recrystallized from ethyl acetate and dichloromethane. MS (m/z, FAB⁺):872.2

### Example 57

### Synthesis of N-(spiro[fluorene-9, 10′-indeno[2,1-b]triphenylene]-6′-yl)-N-p-tolyldibenzo[b, d]furan-4-amine

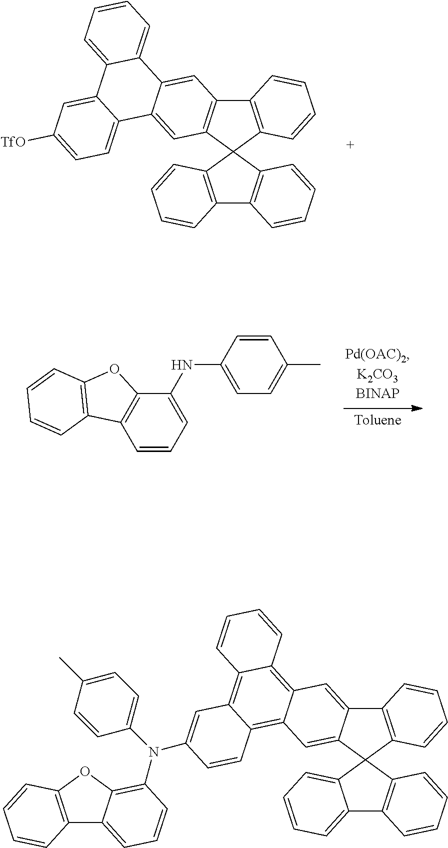 [SMILES: O=S(=O)(Oc1ccc2c(c1)c1ccccc1c1cc3c(cc21)C1(c2ccccc2-c2ccccc21)c1ccccc1-3)C(F)(F)F ; Cc1ccc(Nc2cccc3c2oc2ccccc23)cc1 ; Cc1ccc(N(c2ccc3c(c2)c2ccccc2c2cc4c(cc32)C2(c3ccccc3-c3ccccc32)c2ccccc2-4)c2cccc3c2oc2ccccc23)cc1]

A mixture of 2.7 g (10 mmol) of N-p-tolyldibenzo[b,d]furan-4-amine, 6.1 g (10 mmol) of spiro[fluorene-9,10′-indeno[2,1-b]triphenylene]-6′-yl trifluoromethanesulfonate, 0.09 g (0.4 mmol) of palladium(II)acetate, 0.48 g of BINAP, 3.5 g of potassium carbonate and 150 ml toluene was degassed and placed under nitrogen, and then heated at 110° C. overnight. After finishing the reaction, the mixture was allowed to cool to room temperature. Than 500 ml of MeOH was added, while stirring and the precipitated product was filtered off with suction. To give 7.7 g (yield 67%) of yellow product which was recrystallized from ethyl acetate and hexane. MS (m/z, FAB⁺):737.6

### Example 58

### Synthesis of N,N-dim-tolylspiro[fluorene-9, 10′-indeno[2,1-b]triphenylen]-6′-amine

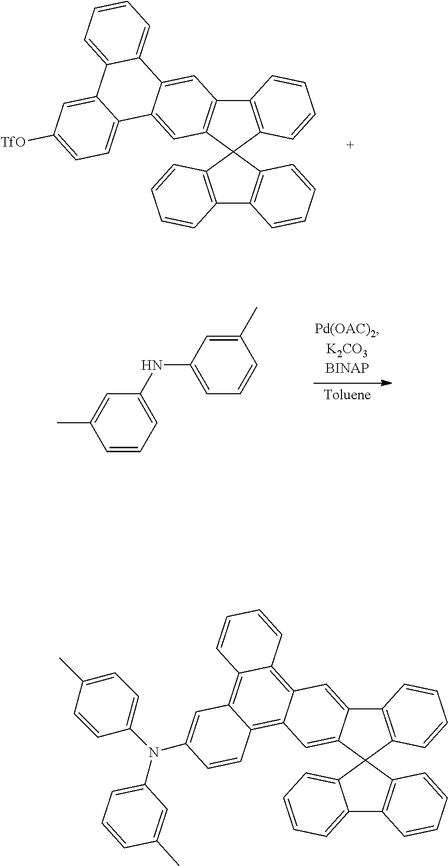

(3-(M-tolylamino)phenyl)methylium instead of N-p-tolyldibenzo[b,d]furan-4-amine, except for using the same method as in synthesis example 57, the desired compound of example 58 (3.2 g, yield=41%) was obtained. MS (m/z, FAB⁺):661.3

### Example 59

### Synthesis of N-(naphthalen-2-yl)-N-phenylspiro[fluorene-9, 10′-indeno[2,1-b]triphenylen]-6′-amine

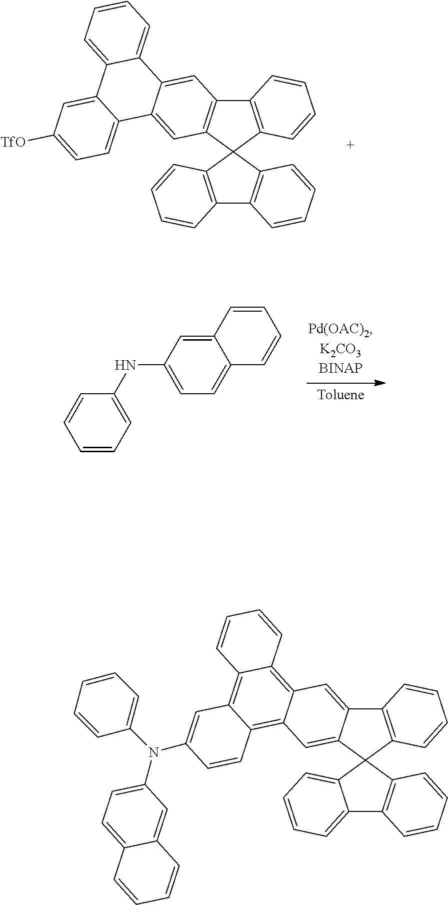 [SMILES: O=S(=O)(Oc1ccc2c(c1)c1ccccc1c1cc3c(cc21)C1(c2ccccc2-c2ccccc21)c1ccccc1-3)C(F)(F)F ; c1ccc(Nc2ccc3ccccc3c2)cc1 ; c1ccc(N(c2ccc3ccccc3c2)c2ccc3c(c2)c2ccccc2c2cc4c(cc32)C2(c3ccccc3-c3ccccc32)c2ccccc2-4)cc1]

N-phenylnaphthalen-2-amine instead of N-p-tolyldibenzo[b,d]furan-4-amine, except for using the same method as in synthesis example 57, the desired compound of example 59 (6.7 g, yield=43%) was obtained. MS (m/z, FAB⁺):683.5

### Example 60

### Synthesis of N,N-di(biphenyl-4-yl)spiro[fluorene-9,10′-indeno[2,1-b]triphenylen]-6′-amine

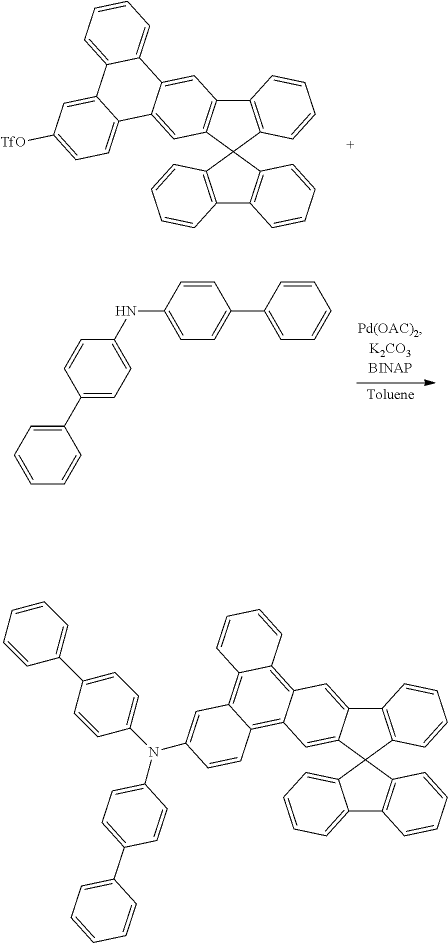 [SMILES: O=S(=O)(Oc1ccc2c(c1)c1ccccc1c1cc3c(cc21)C1(c2ccccc2-c2ccccc21)c1ccccc1-3)C(F)(F)F ; c1ccc(-c2ccc(Nc3ccc(-c4ccccc4)cc3)cc2)cc1 ; c1ccc(-c2ccc(N(c3ccc(-c4ccccc4)cc3)c3ccc4c(c3)c3ccccc3c3cc5c(cc43)C3(c4ccccc4-c4ccccc43)c3ccccc3-5)cc2)cc1]

Dibiphenyl-4-ylamine instead of N-p-tolyldibenzo[b,d]furan-4-amine, except for using the same method as in synthesis example 57, the desired compound of example 60 (6.7 g, yield=43%) was obtained. MS (m/z, FAB⁺):786.2

### Example 61

### Synthesis of N-(biphenyl-4-yl)-N-(9,9-dimethyl-9H-fluoren-2-yl)spiro[fluorene-9, 10′-indeno[2,1-b]triphenylen]-6′-amine

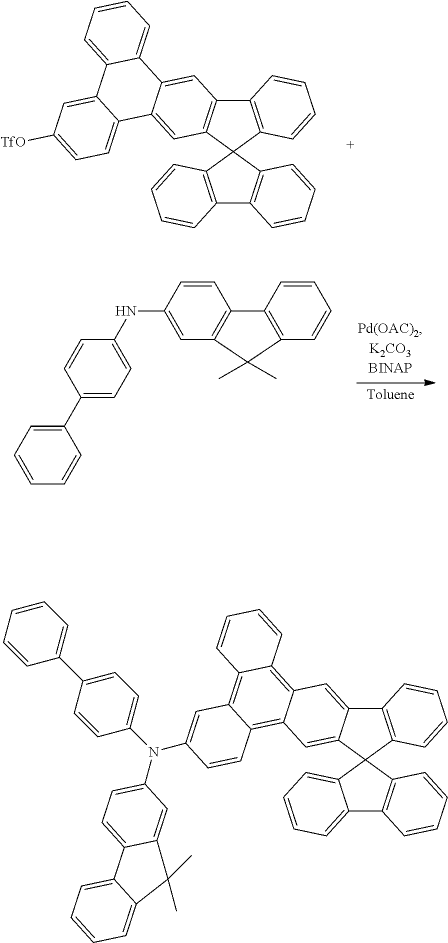 [SMILES: O=S(=O)(Oc1ccc2c(c1)c1ccccc1c1cc3c(cc21)C1(c2ccccc2-c2ccccc21)c1ccccc1-3)C(F)(F)F ; CC1(C)c2ccccc2-c2ccc(Nc3ccc(-c4ccccc4)cc3)cc21 ; CC1(C)c2ccccc2-c2ccc(N(c3ccc(-c4ccccc4)cc3)c3ccc4c(c3)c3ccccc3c3cc5c(cc43)C3(c4ccccc4-c4ccccc43)c3ccccc3-5)cc21]

N-(biphenyl-4-yl)-9,9-dimethyl-91-fluoren-2-amine instead of N-p-tolyldibenzo[b,d]furan-4-amine, except for using the same method as in synthesis example 57, the desired compound of example 61 (6.7 g, yield=43%) was obtained. MS (m/z, FAB⁺):826.5

### Example 62

### Synthesis of N-(biphenyl-4-yl)-N-(4-(dibenzo[b,d]furan-4-yl)phenyl)spiro[fluorene-9,10′-indeno[2,1-b]triphenylen]-6′-amine

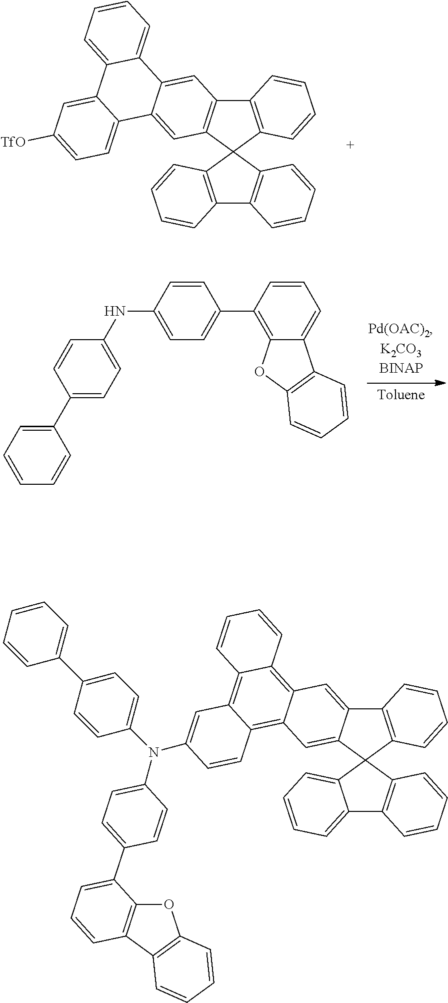 [SMILES: O=S(=O)(Oc1ccc2c(c1)c1ccccc1c1cc3c(cc21)C1(c2ccccc2-c2ccccc21)c1ccccc1-3)C(F)(F)F ; c1ccc(-c2ccc(Nc3ccc(-c4cccc5c4oc4ccccc45)cc3)cc2)cc1 ; c1ccc(-c2ccc(N(c3ccc(-c4cccc5c4oc4ccccc45)cc3)c3ccc4c(c3)c3ccccc3c3cc5c(cc43)C3(c4ccccc4-c4ccccc43)c3ccccc3-5)cc2)cc1]

N-(4-(dibenzo[b,d]furan-4-yl)phenyl) biphenyl-4-amine instead of N-p-tolyldibenzo[b,d]furan-4-amine, except for using the same method as in synthesis example 57, the desired compound of example 62 (2.9 g, yield=31%) was obtained. MS (m/z, FAB⁺):875.8

### Example 63

### Synthesis of 9-(spiro[fluorene-9,10′-indeno[2,1-b]triphenylene]-6′-yl)-9H-carbazole

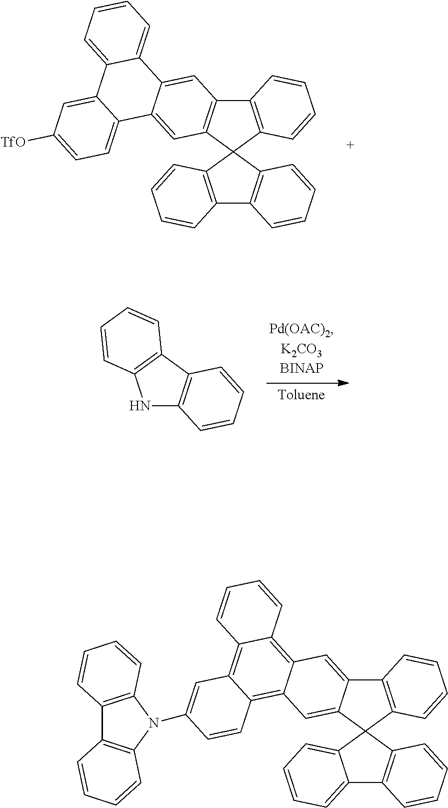 [SMILES: O=S(=O)(Oc1ccc2c(c1)c1ccccc1c1cc3c(cc21)C1(c2ccccc2-c2ccccc21)c1ccccc1-3)C(F)(F)F ; c1ccc2c(c1)[nH]c1ccccc12 ; c1ccc2c(c1)-c1ccccc1C21c2ccccc2-c2cc3c4ccccc4c4cc(-n5c6ccccc6c6ccccc65)ccc4c3cc21]

9H-carbazole instead of 9-phenyl-9H,9′H-3,3′-bicarbazole, except for using the same method as in synthesis example 56, the desired compound of example 63 (4.1 g, yield=36%) was obtained. MS (m/z, FAB⁺):631.3

### Example 64

### Synthesis of 10-(3-bromophenyl)-9,9-dimethyl-9,10-dihydroacridine

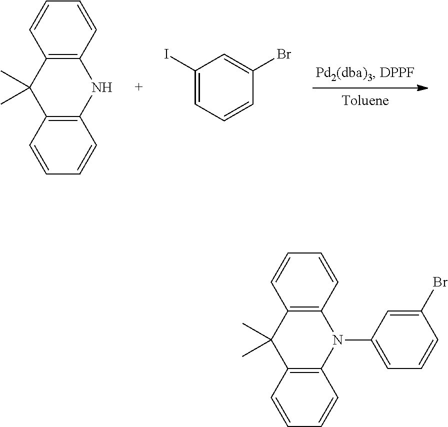 [SMILES: CC1(C)c2ccccc2Nc2ccccc21 ; Brc1cccc(I)c1 ; CC1(C)c2ccccc2N(c2cccc(Br)c2)c2ccccc21]

To a solution of tris(dibenzylideneacetone)dipalladium (1.2 g, 1.32 mmol) and bis(diphenylphosphino)ferrocene (0.88 g, 1.6 mmol) in 300 ml dry toluene under nitrogen atmosphere was added 20.5 g of 1-bromo-3-iodobenzene (72.5 mmol) at room temperature, and the resultant mixture was stirred for 10 minutes, 16 g of sodium tert butoxide and 126 g of 9,9-dimethyl-9, 10-dihydroacridine (60.4 mmol) were added to this solution and stirred at 110° C. overnight under nitrogen. The reaction mixture was poured into 600 ml of water, the organic layer was extracted with ethyl acetate and water, dried with anhydrous magnesium sulfate, the solvent was removed and the product was purified by column using a mixture of hexanes and dichloromethane as eluent to get 8.6 g of product (yield 39%).

### Synthesis of 9,9-dimethyl-10-(3-(4,4,5,5-tetramethyl-1,3,2-dioxaborolan-2-yl)phenyl)-9,10-dihydroacridine

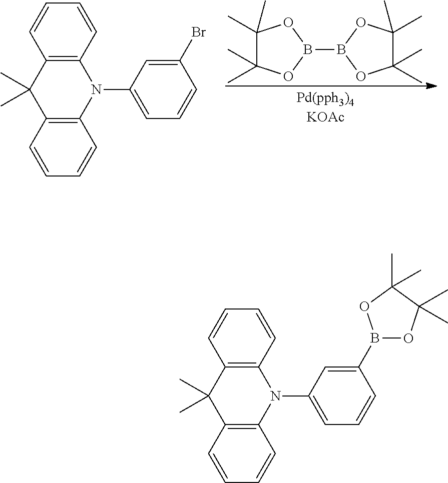

A mixture of 8.6 g (23.6 mmol) of 10-(3-bromophenyl)-9,9-dimethyl-9,10-dihydroacridine, 9 g (35.4 mmol) of bis(pinacolato)diboron, 0.6 g (0.5 mmol) of tetrakis(triphenylphosphine)palladium, 6.9 g (71 mmol) of potassium acetate, and 350 ml of 1,4-dioxane was degassed and placed under nitrogen, and then heated at 90° C. for 6 hours. After finishing the reaction, the mixture was allowed to cool to room temperature. The organic layer was extracted with ethyl acetate and water, dried with anhydrous magnesium sulfate, the solvent was removed and the product was purified by column using a mixture of hexanes and ethyl acetate as eluent to get 6.3 g of product (yield 65%).

### Synthesis of 9,9-dimethyl-10-(3-(spiro[fluorene-9,10′-indeno[1,2-b]triphenylene]-6′-yl)phenyl)-9,10-dihydroacridine

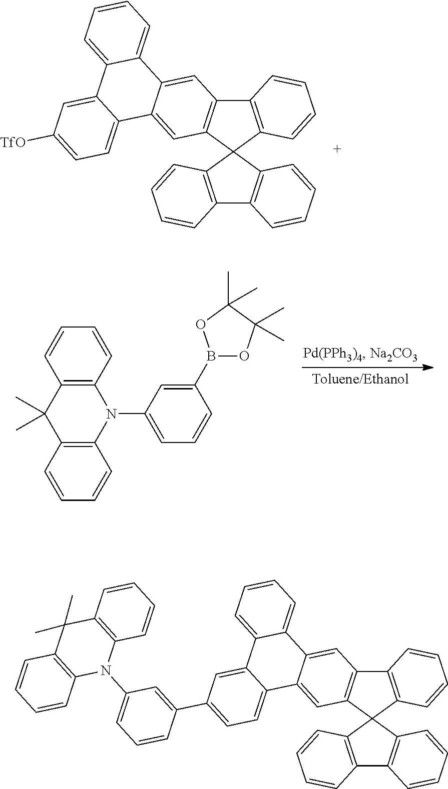

9,9-dimethyl-10-(3-(4,4,5,5-tetramethyl-1,3,2-dioxaborolan-2-yl)phenyl)-9, 10-dihydroacridine instead of 4,6-diphenyl-2-(3-(4,4,5,5-tetramethyl-1,3,2-dioxaborolan-2-yl)phenyl)pyrimidine, except for using the same method as in synthesis example 46, the desired compound of example 64 (2.7 g, yield=41%) was obtained. MS (m/z, FAB⁺):749.3

### Example 65

### Synthesis of 10-(3-bromophenyl)-10H-phenoxazine

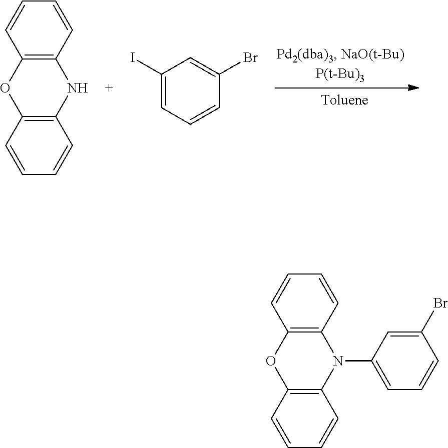 [SMILES: c1ccc2c(c1)Nc1ccccc1O2 ; Brc1cccc(I)c1 ; Brc1cccc(N2c3ccccc3Oc3ccccc32)c1]

A mixture of 32.5 g (114.9 mmol) of 1-bromo-3-iodobenzene, 15.0 g (81.9 mmol) of 10-phenoxazine, 23.6 g (245.8 mmol) of sodium t-butoxide and 2 ml (8.2 mmol) of tri-t-butylphosphine were dissolved in 400 ml of toluene, 1.5 g (1.64 mmol) of Pd₂(dba)₃ was added thereto, and then the mixture was stirred while refluxing overnight. After finishing the reaction, the mixture was allowed to cool to room temperature. The organic layer was extracted with ethyl acetate and water, dried with anhydrous magnesium sulfate, the solvent was removed and the product was purified by column using a mixture of hexanes and ethyl acetate as eluent to get 14.1 g (yield 51%) of product.

### Synthesis of 10-(3-(4,4,5,5-tetramethyl-1,3,2-dioxaborolan-2-yl)phenyl)-10H-phenoxazine

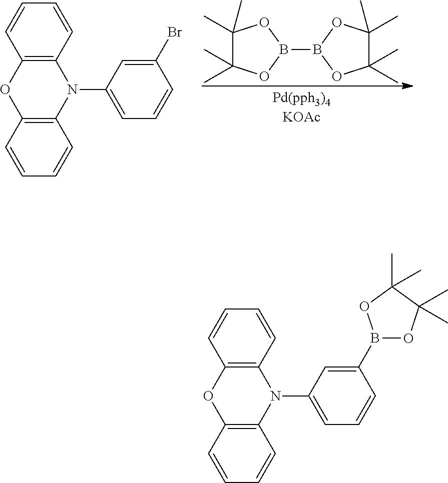

A mixture of 8 g (23.6 mmol) of 10-(3-bromophenyl)-10H-phenoxazine, 9 g (35.4 mmol) of bis(pinacolato)diboron, 0.6 g (0.5 mmol) of tetrakis(triphenylphosphine)palladium, 6.9 g (71 mmol) of potassium acetate, and 350 ml of 1,4-dioxane was degassed and placed under nitrogen, and then heated at 90° C. for 6 hours. After finishing the reaction, the mixture was allowed to cool to room temperature. The organic layer was extracted with ethyl acetate and water, dried with anhydrous magnesium sulfate, the solvent was removed and the product was purified by column using a mixture of hexane and ethyl acetate as eluent to get 3 g of product (yield 34%).

### Synthesis of 10-(3-(spiro[fluorene-9, 10′-indeno[1,2-b]triphenylene]-6′-yl)phenyl)-10H-phenoxazine

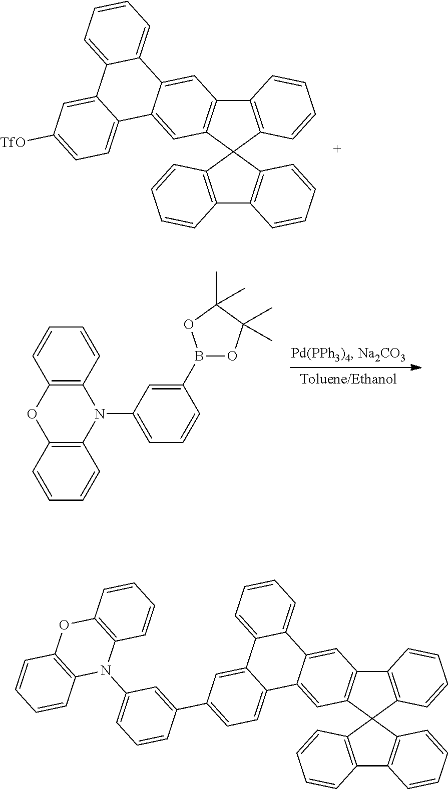 [SMILES: O=S(=O)(Oc1ccc2c(c1)c1ccccc1c1cc3c(cc21)C1(c2ccccc2-c2ccccc21)c1ccccc1-3)C(F)(F)F ; CC1(C)OB(c2cccc(N3c4ccccc4Oc4ccccc43)c2)OC1(C)C ; c1cc(-c2ccc3c(c2)c2ccccc2c2cc4c(cc32)C2(c3ccccc3-c3ccccc32)c2ccccc2-4)cc(N2c3ccccc3Oc3ccccc32)c1]

10-(3-(4,4,5,5-tetramethyl-1,3,2-dioxaborolan-2-yl)phenyl)-10H-phenoxazine instead of 4,6-diphenyl-2-(3-(4,4,5,5-tetramethyl-1,3,2-dioxaborolan-2-yl)phenyl)pyrimidine, except for using the same method as in synthesis example 46, the desired compound of example 65 (1.8 g, yield=57%) was obtained. MS (m/z, FAB⁺):723.3

### Example 66

### Synthesis of 9,9′-(6-chloro-1,3,5-triazine-2,4-diyl)bis(9H-carbazole)

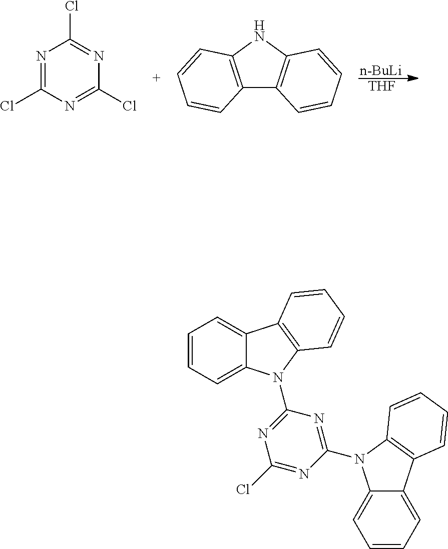

14.2 g (85 mmol) of carbazole was dissolved in 500 ml dry THF under Argon, 50 ml (80 mmol) n-butyllithium (1.6M) was added dropwise to the solution and the mixture was stirred for 30 min., 7.4 g (40 mmol) 2,4,6-trichloro-1,3,5-triazine dissolved in 200 ml dry THF was added dropwise to the solution. The reaction mixture was refluxed for 2 hours. After the solution was cooled to room temperature, 400 ml of water was added. The product was filtered off washed with water, hexane. To give 9.5 g (yield 53%) of product which was recrystallized from ethanol.

### Synthesis of 9,9′-(6-(spiro[fluorene-9, 10′-indeno[2,1-b]triphenylene]-13′-yl)-1,3,5-triazine-2,4-diyl)bis(9H-carbazole)

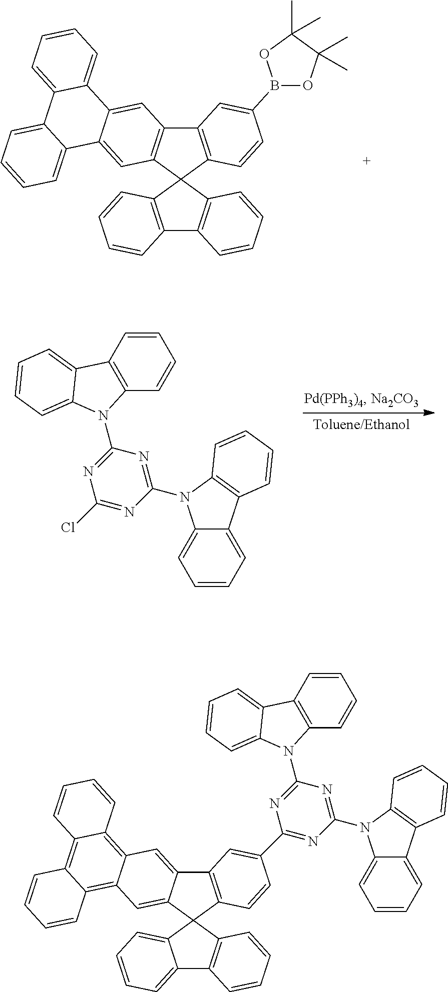 [SMILES: CC1(C)OB(c2ccc3c(c2)-c2cc4c5ccccc5c5ccccc5c4cc2C32c3ccccc3-c3ccccc32)OC1(C)C ; Clc1nc(-n2c3ccccc3c3ccccc32)nc(-n2c3ccccc3c3ccccc32)n1 ; c1ccc2c(c1)-c1ccccc1C21c2ccc(-c3nc(-n4c5ccccc5c5ccccc54)nc(-n4c5ccccc5c5ccccc54)n3)cc2-c2cc3c4ccccc4c4ccccc4c3cc21]

9,9′-(6-chloro-1,3,5-triazine-2,4-diyl)bis(9H-carbazole) instead of 2-chloro-1,10-phenanthroline, except for using the same method as in synthesis example 32, the desired compound of example 66 (2.3 g, yield=31%) was obtained. MS (m/z, FAB⁺):875.4

### Example 67

### Synthesis of 9-(4,6-dichloropyrimidin-2-yl)-9H-carbazole

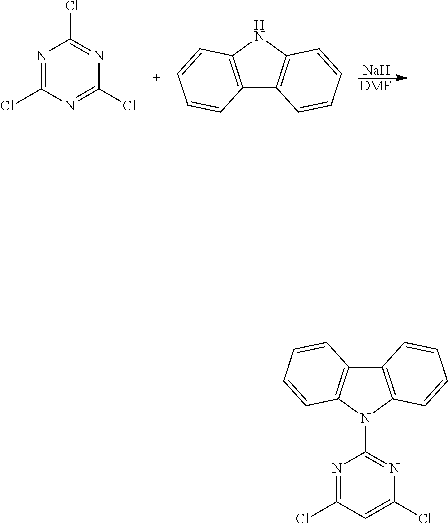

14.2 g (85 mmol) of carbazole was dissolved in 150 ml DMF under Argon, 3.1 g (127.5 mmol) of NaH dissolved in 100 ml DMF was added dropwise to the solution and the mixture was stirred for 60 min, 18.3 g (100 mmol) 2,4,6-trichloropyrimidine dissolved in 300 ml DMF was added dropwise to the solution. The reaction mixture was stirred for 16 hours. After finishing the reaction 800 ml of ice water was added. The product was filtered off washed with water, hexane. To give 10.9 g (yield 41%) of product which was recrystallized from ethanol.

### Synthesis of 9-(4-chloro-6-phenylpyrimidin-2-yl)-9H-carbazole

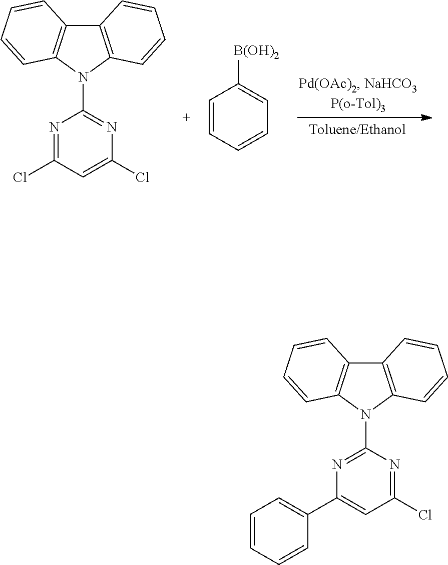 [SMILES: Clc1cc(Cl)nc(-n2c3ccccc3c3ccccc32)n1 ; OB(O)c1ccccc1 ; Clc1cc(-c2ccccc2)nc(-n2c3ccccc3c3ccccc32)n1]

10.9 g (34.7 mmol) of 9-(4,6-dichloropyrimidin-2-yl)-9H-carbazole, 4.2 g (34.7 mmol) of phenylboronic acid and 26 ml of 2M NaHCO₃ solution are suspended in 100 ml of ethylene glycol dimethyl ether. 0.3 g (1.4 mmol) of Pd(OAc)₂ and 0.52 g (1.7 mmol) of P (o-Tol)₃ was added to the suspension, and the reaction mixture was heated under reflux overnight. After finishing the reaction, the mixture was allowed to cool to room temperature. The reaction mixture was extracted with ethyl acetate and water, dried with anhydrous magnesium sulfate and evaporated in a rotary evaporator. The product was purified by column using a mixture of hexane and ethyl acetate as eluent to get 7.8 g (yield 63%).

### Synthesis of 2,4-diphenyl-6-(spiro[fluorene-9,10′-indeno[1,2-b]triphenylene]-12′-yl)-1,3,5-triazine

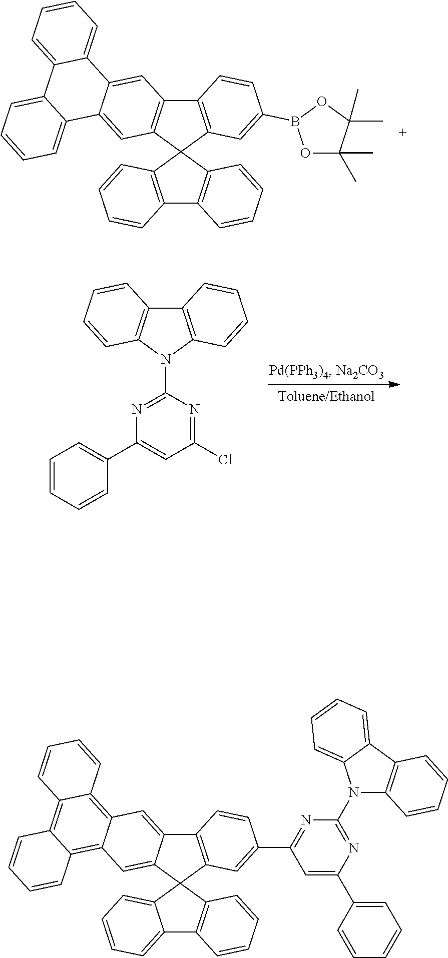 [SMILES: CC1(C)OB(c2ccc3c(c2)C2(c4ccccc4-c4ccccc42)c2cc4c5ccccc5c5ccccc5c4cc2-3)OC1(C)C ; Clc1cc(-c2ccccc2)nc(-n2c3ccccc3c3ccccc32)n1 ; c1ccc(-c2cc(-c3ccc4c(c3)C3(c5ccccc5-c5ccccc53)c3cc5c6ccccc6c6ccccc6c5cc3-4)nc(-n3c4ccccc4c4ccccc43)n2)cc1]

9-(4-chloro-6-phenylpyrimidin-2-yl)-9H-carbazole instead of 2-chloro-4,6-diphenyl-1,3,5-triazine, except for using the same method as in synthesis example 33, the desired compound of example 67 (3.6 g, yield=44%) was obtained. MS (m/z, FAB⁺):785.3

### Example 68

### Synthesis of 9-(4-phenyl-6-(spiro[fluorene-9,10′-indeno[2,1-b]triphenylene]-13′-yl)pyrimidin-2-yl)-9H-carbazole

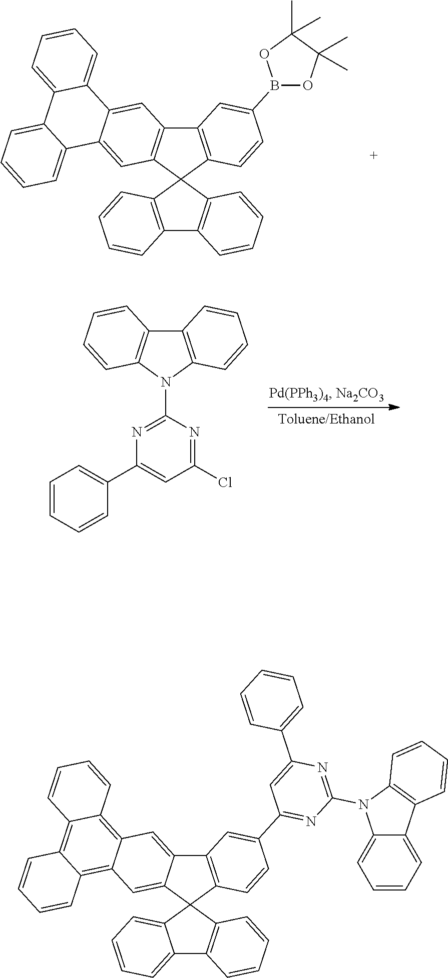 [SMILES: CC1(C)OB(c2ccc3c(c2)-c2cc4c5ccccc5c5ccccc5c4cc2C32c3ccccc3-c3ccccc32)OC1(C)C ; Clc1cc(-c2ccccc2)nc(-n2c3ccccc3c3ccccc32)n1 ; c1ccc(-c2cc(-c3ccc4c(c3)-c3cc5c6ccccc6c6ccccc6c5cc3C43c4ccccc4-c4ccccc43)nc(-n3c4ccccc4c4ccccc43)n2)cc1]

9-(4-chloro-6-phenylpyrimidin-2-yl)-9H-carbazole instead of 2-chloro-1, 10-phenanthroline, except for using the same method as in synthesis example 32, the desired compound of example 68 (2.7 g, yield=44%) was obtained. MS (m/z, FAB⁺):786.1

### Example 69

### Synthesis of 9-(4,6-dichloro-1,3,5-triazin-2-yl)-9H-carbazole

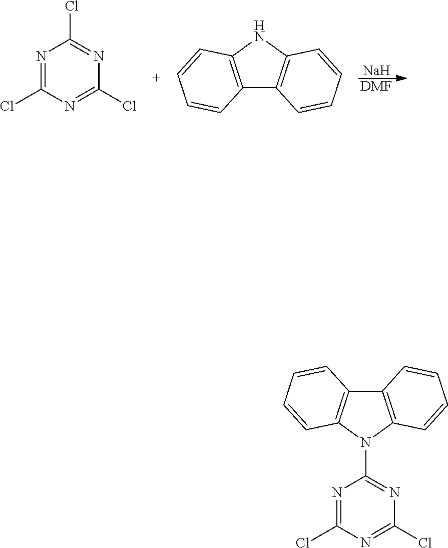

14.2 g (85 mmol) of carbazole was dissolved in 150 ml DMF under Argon, 3.1 g (127.5 mmol) of NaH dissolved in 100 ml DMF was added dropwise to the solution and the mixture was stirred for 60 min, 18.5 g (100 mmol) 2,4,6-trichloro-1,3,5-triazine dissolved in 300 ml DMF was added dropwise to the solution. The reaction mixture was stirred for 16 hours. After finishing the reaction 800 ml of ice water was added. The product was filtered off washed with water, hexane. To give 14.2 g (yield 53%) of product which was recrystallized from ethanol.

### Synthesis of 9-(4-chloro-6-phenyl-1,3,5-triazin-2-yl)-9H-carbazole

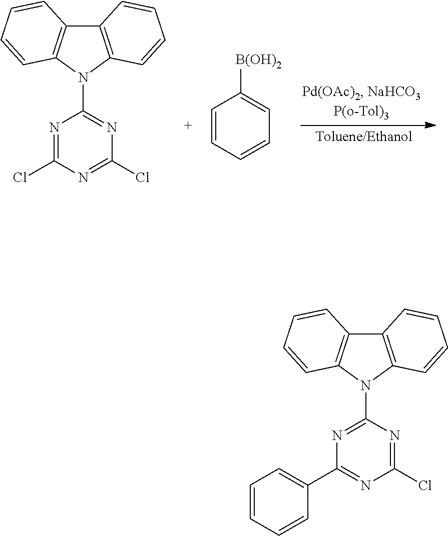 [SMILES: Clc1nc(Cl)nc(-n2c3ccccc3c3ccccc32)n1 ; OB(O)c1ccccc1 ; Clc1nc(-c2ccccc2)nc(-n2c3ccccc3c3ccccc32)n1]

14.2 g (45 mmol) of 9-(4,6-dichloro-1,3,5-triazin-2-yl)-9H-carbazole, 5.5 g (45 mmol) of phenylboronic acid and 45 ml of 2M NaHCO₃ solution are suspended in 150 ml of ethylene glycol dimethyl ether. 0.38 g (1.8 mmol) of Pd(OAc)₂ and 0.68 g (2.2 mmol) of P (o-Tol)₃ was added to the suspension, and the reaction mixture was heated under reflux overnight. After finishing the reaction, the mixture was allowed to cool to room temperature. The reaction mixture was extracted with ethyl acetate and water, dried with anhydrous magnesium sulfate and evaporated in a rotary evaporator. The product was purified by column using a mixture of hexane and ethyl acetate as eluent to get 7.5 g (yield 47%).

### Synthesis of 9-(4-phenyl-6-(spiro[fluorene-9,10′-indeno[2,1-b]triphenylene]-13′-yl)-1,3,5-triazin-2-yl)-9H-carbazole

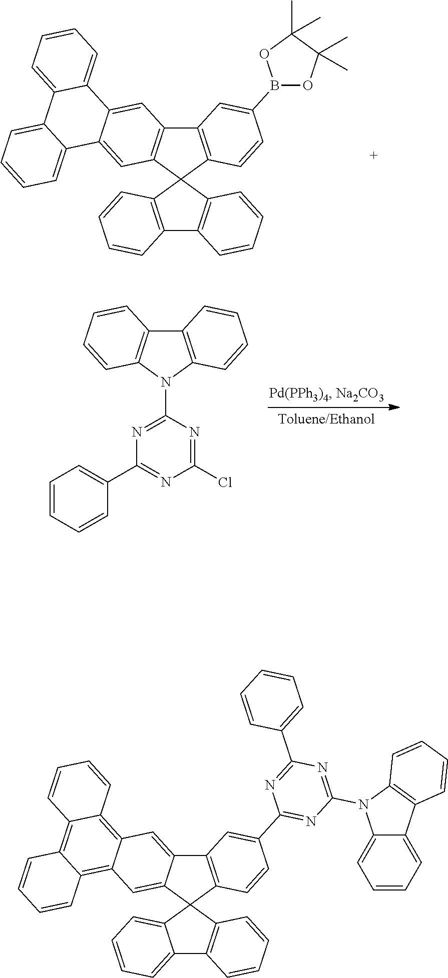 [SMILES: CC1(C)OB(c2ccc3c(c2)-c2cc4c5ccccc5c5ccccc5c4cc2C32c3ccccc3-c3ccccc32)OC1(C)C ; Clc1nc(-c2ccccc2)nc(-n2c3ccccc3c3ccccc32)n1 ; c1ccc(-c2nc(-c3ccc4c(c3)-c3cc5c6ccccc6c6ccccc6c5cc3C43c4ccccc4-c4ccccc43)nc(-n3c4ccccc4c4ccccc43)n2)cc1]

9-(4-chloro-6-phenyl-1,3,5-triazin-2-yl)-9H-carbazole instead of 2-chloro-1,10-phenanthroline, except for using the same method as in synthesis example 32, the desired compound of example 69 (3.5 g, yield=61%) was obtained. MS (m/z, FAB⁺):786.3

### Example 70

### Synthesis of 9-(4-phenyl-6-(spiro[fluorene-9,10′-indeno[2,1-b]triphenylene]-12′-yl)-1,3,5-triazin-2-yl)-9H-carbazole

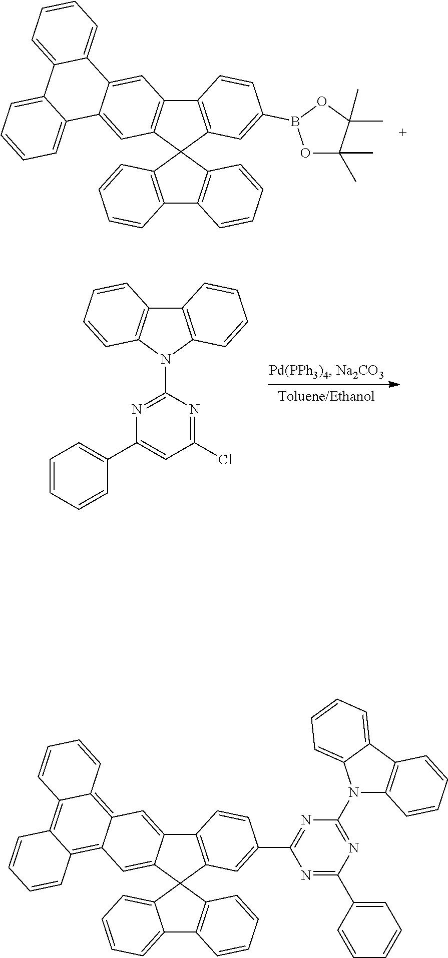 [SMILES: CC1(C)OB(c2ccc3c(c2)C2(c4ccccc4-c4ccccc42)c2cc4c5ccccc5c5ccccc5c4cc2-3)OC1(C)C ; Clc1cc(-c2ccccc2)nc(-n2c3ccccc3c3ccccc32)n1 ; c1ccc(-c2nc(-c3ccc4c(c3)C3(c5ccccc5-c5ccccc53)c3cc5c6ccccc6c6ccccc6c5cc3-4)nc(-n3c4ccccc4c4ccccc43)n2)cc1]

9-(4-chloro-6-phenyl-1,3,5-triazin-2-yl)-9H-carbazole instead of 2-chloro-4,6-diphenyl-1,3,5-triazine, except for using the same method as in synthesis example 33, the desired compound of example 70 (5.1 g, yield=49%) was obtained. MS (m/z, FAB⁺):786.3

### Example 71

### Synthesis of 9-(4-phenyl-6-(3-(4,4,5,5-tetramethyl-1,3,2-dioxaborolan-2-yl)phenyl)-1,3,5-triazin-2-yl)-9H-carbazole

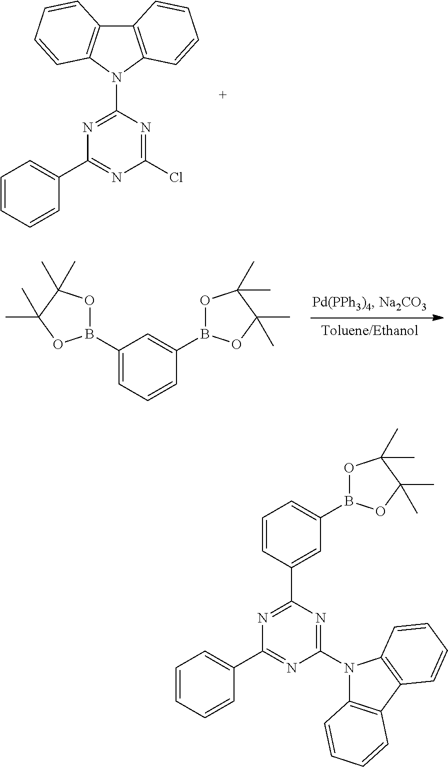

A mixture of 16.5 g (50 mmol) of 1,3-bis(4,4,5,5-tetramethyl-1, 3,2-dioxaborolan-2-yl)benzene, 17.8 g (50 mmol) of 9-(4-chloro-6-phenyl-1,3,5-triazin-2-yl)-9H-carbazole, 1.15 g (1 mmol) of tetrakis(triphenylphosphine) palladium, 37 ml of 2M Na₂CO₃, 50 ml of EtOH and 100 ml toluene was degassed and placed under nitrogen, and then heated at 75° C. overnight. After finishing the reaction, the mixture was allowed to cool to room temperature. The reaction mixture was extracted with ethyl acetate and water, dried with anhydrous magnesium sulfate, the solvent was evaporated in vacuum. The residue was purified by column using a mixture of hexane and 30% ethyl acetate as eluent to get 8.9 g of product (yield 34%)

### 9-(4-phenyl-6-(3-(spiro[fluorene-9,10′-indeno[2,1-b]triphenylene]-13′-yl)phenyl)-1,3,5-triazin-2-yl)-9H-carbazole

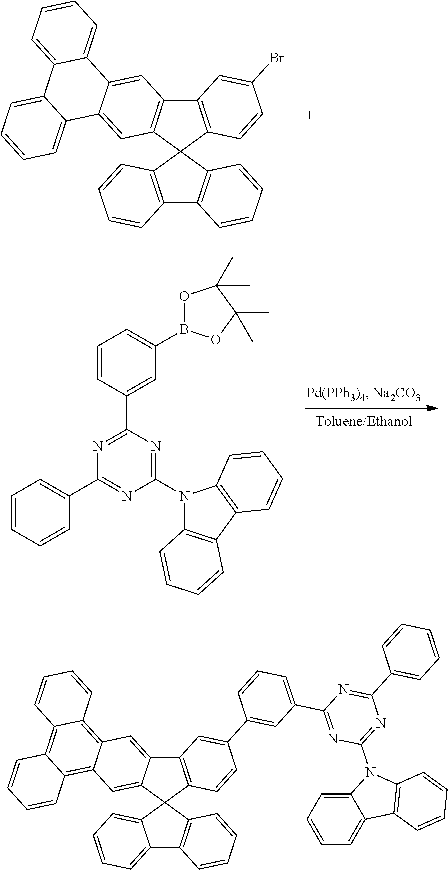

9-(4-phenyl-6-(3-(4,4,5,5-tetramethyl-1,3,2-dioxaborolan-2-yl)phenyl)-1,3,5-triazin-2-yl)-9H-carbazole instead of 2-(3-(dibenzo[b,d]furan-4-yl)phenyl)-4,4,5,5-tetramethyl-1,3,2-dioxaborolane, except for using the same method as in synthesis example 2, the desired compound of example 71 (3.5 g, yield=61%) was obtained. MS (m/z, FAB⁺):862.4

### Example 72

### Synthesis of 9-(4-phenyl-6-(3-(spiro[fluorene-9, 10′-indeno[2,1-b]triphenylene]-6′-yl)phenyl)-1,3,5-triazin-2-yl)-9H-carbazole

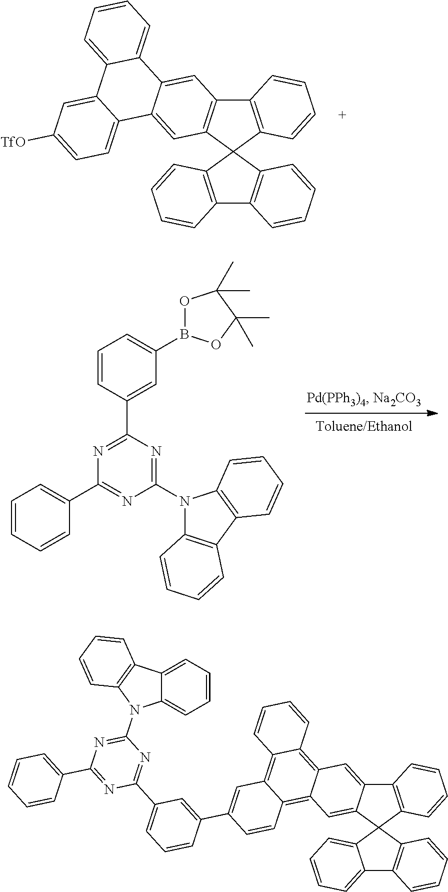 [SMILES: O=S(=O)(Oc1ccc2c(c1)c1ccccc1c1cc3c(cc21)C1(c2ccccc2-c2ccccc21)c1ccccc1-3)C(F)(F)F ; CC1(C)OB(c2cccc(-c3nc(-c4ccccc4)nc(-n4c5ccccc5c5ccccc54)n3)c2)OC1(C)C ; c1ccc(-c2nc(-c3cccc(-c4ccc5c(c4)c4ccccc4c4cc6c(cc54)C4(c5ccccc5-c5ccccc54)c4ccccc4-6)c3)nc(-n3c4ccccc4c4ccccc43)n2)cc1]

9-(4-phenyl-6-(3-(4,4,5,5-tetramethyl-1,3,2-dioxaborolan-2-yl)phenyl)-1,3,5-triazin-2-yl)-9H-carbazole instead of 2-(3-(4,4,5,5-tetramethyl-1,3,2-dioxaborolan-2-yl)phenyl)-1,10-phenanthroline, except for using the same method as in synthesis example 44, the desired compound of example 72 (2.7 g, yield=41%) was obtained. MS (m/z, FAB⁺):862.4

### Example 73

### Synthesis of 2-bromo-7-(5-methoxybiphenyl-2-yl)-9,9′-spirobi[fluorene]

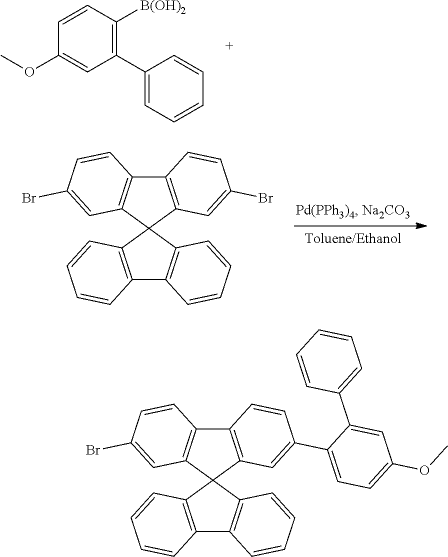

A mixture of 47.4 g (100 mmol) of 2,7-dibromo-9,9′-spirobi[fluorene], 25.1 g (110 mmol) of 5-methoxybiphenyl-2-ylboronicacid, 0.24 g (0.2 mmol) of tetrakis(triphenylphosphine)palladium, 100 ml of 2M Na₂CO₃, 125 ml of EtOH and 250 ml toluene was degassed and placed under nitrogen, and then heated at 110° C. for 16 hours. After finishing the reaction, the mixture was allowed to cool to room temperature. The reaction mixture was extracted with ethyl acetate and water, dried with anhydrous magnesium sulfate, the solvent was evaporated in vacuum. The residue was purified by column using a mixture of hexanes and dichloromethane as eluent to get 35.8 g of product (yield 62%)

### Synthesis of 12′-bromo-6′-methoxyspiro[fluorene-9,10′-indeno[1,2-b]triphenylene]

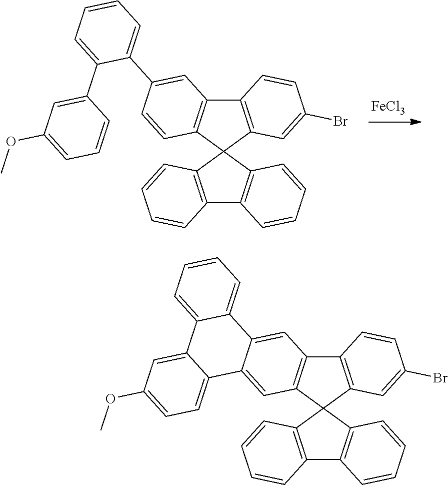

In a 1000 ml three-necked flask that had been degassed and filled with nitrogen, 11.3 g (19.5 mmol) of 2-bromo-7-(5-methoxybiphenyl-2-yl)-9,9′-spirobi[fluorene] was dissolved in anhydrous dichloromethane (450 ml), 16.2 g (100 mmol) iron (III) chloride was then added, and the mixture was stirred 5 minutes. Methanol 100 ml were added to the mixture and the organic layer was separated and the solvent removed in vacuo. The residue was purified by column chromatography on silica (hexane-dichloromethane) afforded a white solid 8.5 g (76%).

### Synthesis of 2-(6′-methoxyspiro[fluorene-9,10′-indeno[1,2-b]triphenylene]-12′-yl)-4,4,5,5-tetramethyl-1,3,2-dioxaborolane

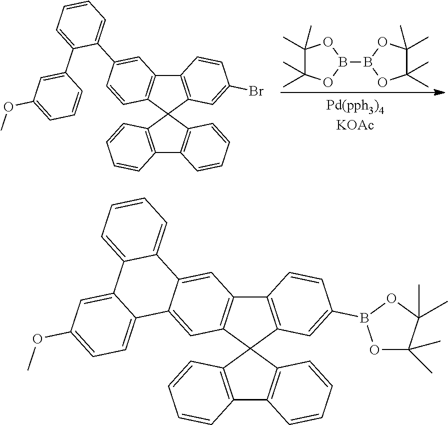

A mixture of 5.8 g (10.1 mmol) of 12′-bromo-6′-methoxyspiro[fluorene-9, 10′-indeno[1,2-b]triphenylene], 2.6 g (15.1 mmol) of bis(pinacolato)diboron, 0.23 (0.2 mmol) of tetrakis(triphenylphosphine) palladium, 3 g (30.3 mmol) of potassium acetate, and 60 ml of 1,4-dioxane was degassed and placed under nitrogen, and then heated at 90° C. for 4 hours. After finishing the reaction, the mixture was allowed to cool to room temperature. The organic layer was extracted with ethyl acetate and water, dried with anhydrous magnesium sulfate, the solvent was removed and the product was purified by column using a mixture of hexanes and dichloromethane as eluent to get 4. Ig of product (yield 65%).

### Synthesis of 2-(6′-methoxyspiro[fluorene-9,10′-indeno[1,2-b]triphenylene]-12′-yl)-4,6-diphenyl-1,3,5-triazine

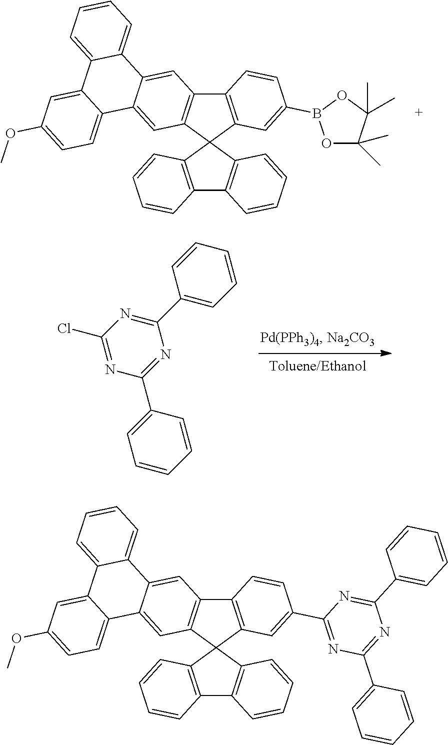

A mixture of 4.2 g (6.8 mmol) of 2-(6′-methoxyspiro[fluorene-9, 10′-indeno[1,2-b]triphenylene]-12′-yl)-4,4,5,5-tetramethyl-1,3,2-dioxaborolane, 1.6 g (10 mmol) of 2-chloro-4,6-diphenyl-1,3,5-triazine, 0.17 g (0.15 mmol) of tetrakis(triphenylphosphine)palladium, 10 ml of 2M Na₂CO₃, 20 ml of EtOH and 40 ml toluene was degassed and placed under nitrogen, and then heated at 90° C. overnight. After finishing the reaction, the mixture was allowed to cool to room temperature. The organic layer was extracted with ethyl acetate and water, dried with anhydrous magnesium sulfate, the solvent was removed and the product was purified by column using a mixture of hexanes and dichloromethane as eluent to get 3.2 g of product (yield 65%).

### Synthesis of 12′-(4,6-diphenyl-1,3,5-triazin-2-yl)spiro[fluorene-9,10′-indeno[2,1-b]triphenylen]-6′-ol

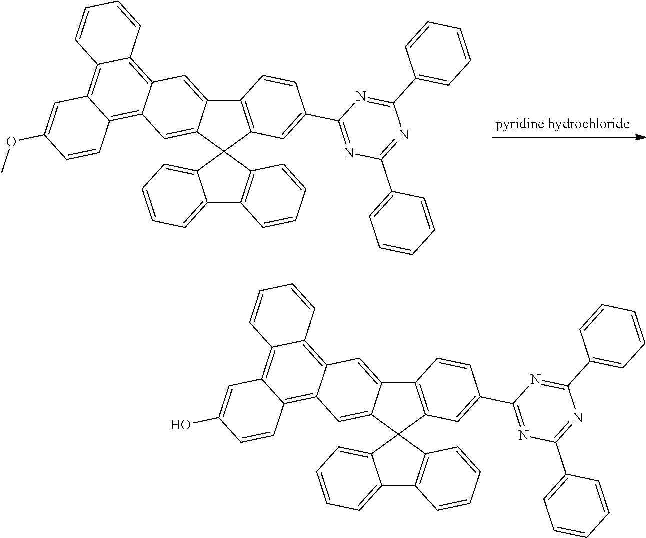 [SMILES: COc1ccc2c(c1)c1ccccc1c1cc3c(cc21)C1(c2ccccc2-c2ccccc21)c1cc(-c2nc(-c4ccccc4)nc(-c4ccccc4)n2)ccc1-3 ; Oc1ccc2c(c1)c1ccccc1c1cc3c(cc21)C1(c2ccccc2-c2ccccc21)c1cc(-c2nc(-c4ccccc4)nc(-c4ccccc4)n2)ccc1-3]

A mixture of 3.2 g (4.4 mmol) of 2-(6′-methoxyspiro[fluorine-9,10′-indeno[1,2-b]triphenylene]-12′-yl)-4,6-diphenyl-1,3,5-triazine, 7.8 g (67.5 mmol) of pyridine hydrochloride, was degassed and placed under nitrogen, and then heated at 220° C. for 6 h, the mixture was allowed to cool to room temperature and water was added. The resulting solid was filtered off, washed with water, and dried under high vacuum to give product 2.9 g (4.1 mmol, 93%)

### Synthesis of 12′-(4,6-diphenyl-1,3,5-triazin-2-yl)spiro[fluorine-9,10′-indeno[2,1-b]triphenylene]-6′-yltrifluoromethanesulfonate

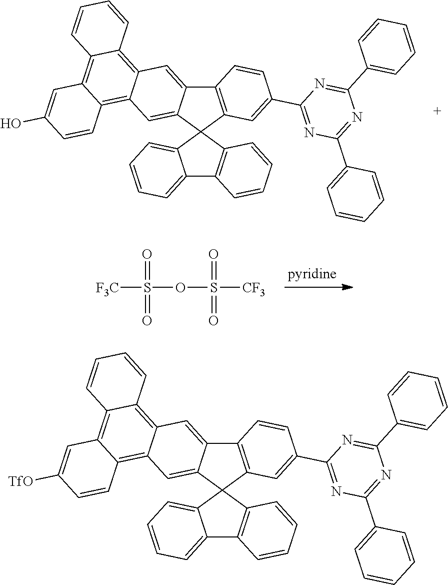

In a 1000 ml three-necked flask that had been degassed and filled with nitrogen, 2.9 g (4.1 mmol) of 12′-(4,6-diphenyl-1,3,5-triazin-2-yl)spiro[fluorene-9,10′-indeno[2,1-b]triphenylen]-6′-ol was dissolved in anhydrous dichloromethane (80 ml), 4 ml pyridine was then added, and the mixture was cooled in an ice salt bath. 1.4 ml (8.2 mmol) trifluoromethanesulfonic anhydride in 10 ml dichloromethane was added dropwise to the solution under nitrogen, the reaction was allowed to proceed for 6 hours and quenched by adding methanol and water. The resulting solid was filtered off, washed with water, methanol and dichloromethane, the residue product was recrystallized from toluene. 2.5 g (3 mmol, 72%) product was obtained.

### Synthesis of 9-(12′-(4,6-diphenyl-1,3,5-triazin-2-yl)spiro[fluorene-9,10′-indeno[2,1-b]triphenylene]-6′-yl)-9H-carbazole

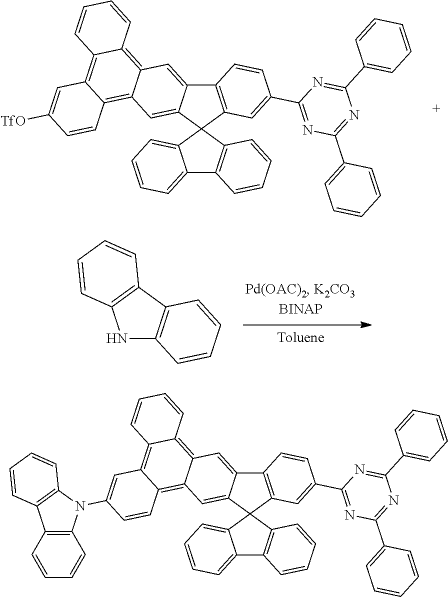

A mixture of 0.5 g (3 mmol) of carbazole, 2.5 g (3 mmol) of 13′-(4,6-diphenyl-1,3,5-triazin-2-yl)spiro[fluorene-9,10′-indeno[2,1-b]triphenylene]-6′-yl trifluoromethanesulfonate, 0.05 g (0.2 mmol) of palladium(II)acetate, 0.24 g of BINAP, 2 g of potassium carbonate and 50 ml toluene was degassed and placed under nitrogen, and then heated at 110° C. overnight. After finishing the reaction, the mixture was allowed to cool to room temperature. Than 200 ml of MeOH was added, while stirring and the precipitated product was filtered off with suction. To give 1.3 g (yield 52%) of yellow product which was recrystallized from ethyl acetate and dichloromethane. MS (m/z, FAB⁺):862.4

### Example 74

### Synthesis of 6′-methoxy-N,N-dim-tolylspiro[fluorene-9,10′-indeno[1,2-b]triphenylen]-12′-amine

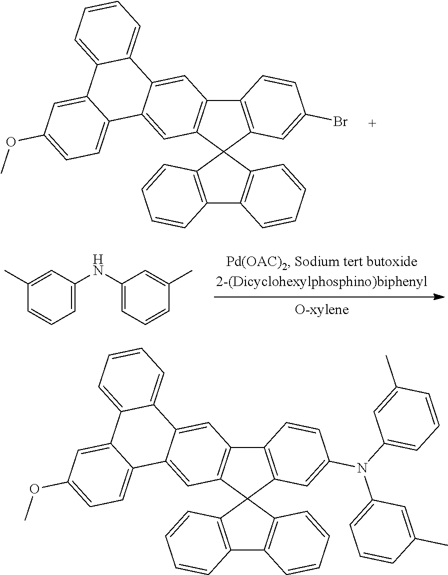

A mixture of 5.8 g (10.1 mmol) 12′-bromo-6′-methoxyspiro[fluorene-9, 10′-indeno[1,2-b]triphenylene],3 g (15.1 mmol) of dim-tolylamine, 0.05 g (0.2 mmol) of palladium(II)acetate, 0.15 g (0.4 mmol) of 2-(dicyclohexyl phosphino)biphenyl, 2 g (20 mmol) of sodium tert-butoxide and 100 ml of toluene was refluxed under nitrogen overnight. After finishing the reaction, the solution was filtered at 100° C., to receive the filtrate, and the filtrate was added to 1 L MeOH, while stirring and the precipitated product was filtered off with suction. To give 5 g (yield 71%) of yellow product which was recrystallized from toluene.

### Synthesis of 12′-(dim-tolylamino)spiro[fluorene-9,10′-indeno[2,1-b]triphenylen]-6′-ol

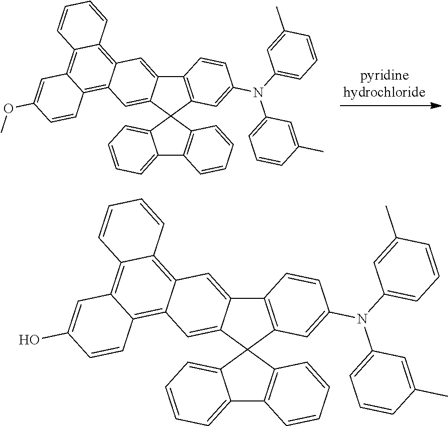

A mixture of 5 g (7.2 mmol) of 6′-methoxy-N,N-dim-tolylspiro[fluorene-9,10′-indeno[1,2-b]triphenylen]-12′-a mine, 12.9 g (113 mmol) of pyridine hydrochloride, was degassed and placed under nitrogen, and then heated at 220° C. for 6 h, the mixture was allowed to cool to room temperature and water was added. The resulting solid was filtered off, washed with water, and dried under high vacuum to give product 4.3 g (6.3 mmol, 87%)

### Synthesis of 12′-(dim-tolylamino)spiro[fluorene-9,10′-indeno[2,1-b]triphenylene]-6′-yl trifluoromethanesulfonate

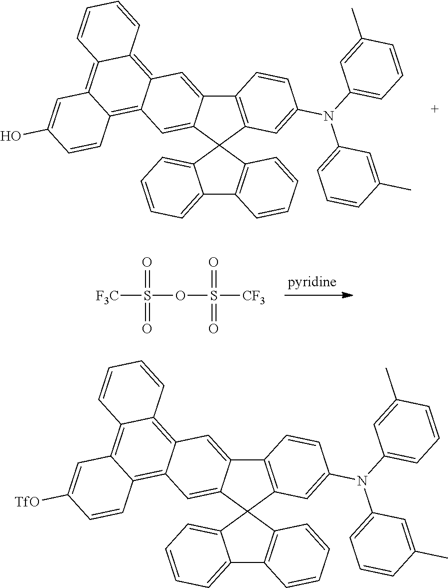

In a 1000 ml three-necked flask that had been degassed and filled with nitrogen, 4.3 g (6.3 mmol) of 12′-(dim-tolylamino)spiro[fluorene-9,10′-indeno[2,1-b]triphenylen]-6′-ol was dissolved in anhydrous dichloromethane (120 ml), 6 ml pyridine was then added, and the mixture was cooled in an ice salt bath. 2.1 ml (12.3 mmol)trifluoromethanesulfonic anhydride in 15 ml dichloromethane was added dropwise to the solution under nitrogen, the reaction was allowed to proceed for 6 hours and quenched by adding methanol and water. The resulting solid was filtered off, washed with water, methanol and dichloromethane, the residue product was recrystallized from toluene. 4 g (3 mmol, 79%) product was obtained.

### Synthesis of N6′,N6′,N12′,N12′-tetram-tolylspiro[fluorene-9,10′-indeno[2,1-b]triphenylene]-6′,12′-diamine

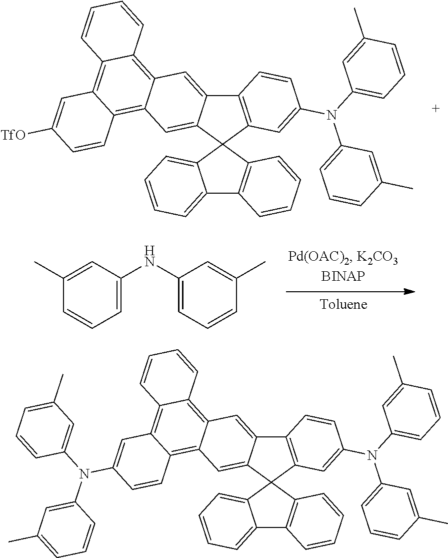

A mixture of 1 g (6 mmol) of dim-tolylamine, 2.4 g (3 mmol) of 12′-(dim-tolylamino)spiro[fluorene-9,10′-indeno[2,1-b]triphenylene]-6′-yl trifluoromethanesulfonate, 0.1 g (0.4 mmol) of palladium(II)acetate, 0.48 g of BINAP, 4 g of potassium carbonate and 50 ml toluene was degassed and placed under nitrogen, and then heated at 110° C. overnight. After finishing the reaction, the mixture was allowed to cool to room temperature. Than 200 ml of MeOH was added, while stirring and the precipitated product was filtered off with suction. To give 0.8 g (yield 31%) of yellow product which was recrystallized from ethyl acetate and dichloromethane. MS (m/z, FAB⁺):858.1

### General Method of Producing Organic EL Device

ITO-coated glasses with 9˜12 ohm/square in resistance and 120˜160 nm in thickness are provided (hereinafter ITO substrate) and cleaned in a number of cleaning steps in an ultrasonic bath (e.g. detergent, deionized water). Before vapor deposition of the organic layers, cleaned ITO substrates are further treated by UV and ozone. All pre-treatment processes for ITO substrate are under clean room (class 100).

These organic layers are applied onto the ITO substrate in order by vapor deposition in a high-vacuum unit (10⁻⁷ Torr), such as: resistively heated quartz boats. The thickness of the respective layer and the vapor deposition rate (0.1˜0.3 nm/sec) are precisely monitored or set with the aid of a quartz-crystal monitor. It is also possible, as described above, for individual layers to consist of more than one compound, i.e. in general a host material doped with a dopant material. This is achieved by co-vaporization from two or more sources.

Dipyrazino[2,3-f:2,3-]quinoxaline-2,3,6,7,10,11-hexacarbonitrile (HAT-CN) is used as hole injection layer in this organic EL device. N,N-Bis(naphthalene-1-yl)-N,N-bis(phenyl)-benzidine (NPB) is most widely used as the hole transporting layer. 10,10-Dimethyl-12-(4-(pyren-1-yl)phenyl)-10H-indeno[1,2-b]triphenylene (PT-312, US20140175384) is used as blue emitting host and N1,N1,N6,N6-tetram-tolylpyrene-1,6-diamine (D1) is used as blue guest. 2-(10,10-dimethyl-10H-indeno[2,1-b]triphenylen-13-yl)-9-phenyl-1,10-phenanthroline is used as electron transporting material (ET1) to co-deposit with 5% Li,2-(10,10-dimethyl-10H-indeno[2,1-b]triphenylen-12-yl)-4,6-diphenyl-1,3,5-triazine is used as electron transporting material (ET2) to co-deposit with 8-hydroxyquinolato-lithium (LiQ) in organic EL device. Bis(2-methyl-8-quinolinolate)-4-(phenylphenolato)aluminium (BAlq) is used as hole blocking material (HBM) and phosphorescent host for phosphorescent system. Bis(2-phenylpyridinato)(2,4-diphenylpyridinato) iridium (III)(D1) are used as phosphorescent dopant. The prior art of OLED materials for producing standard organic EL device control and comparable material in this invention shown its chemical structure as following:

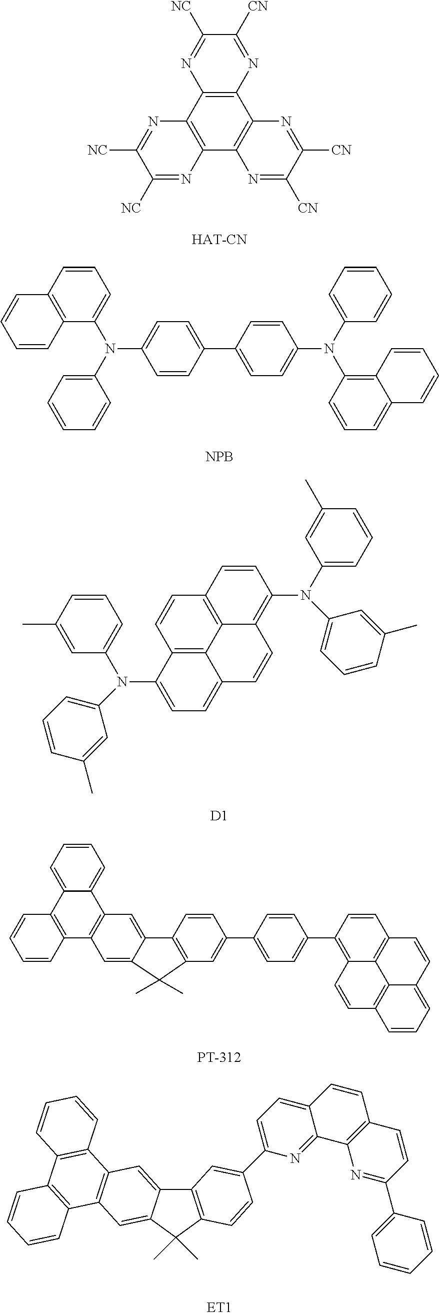

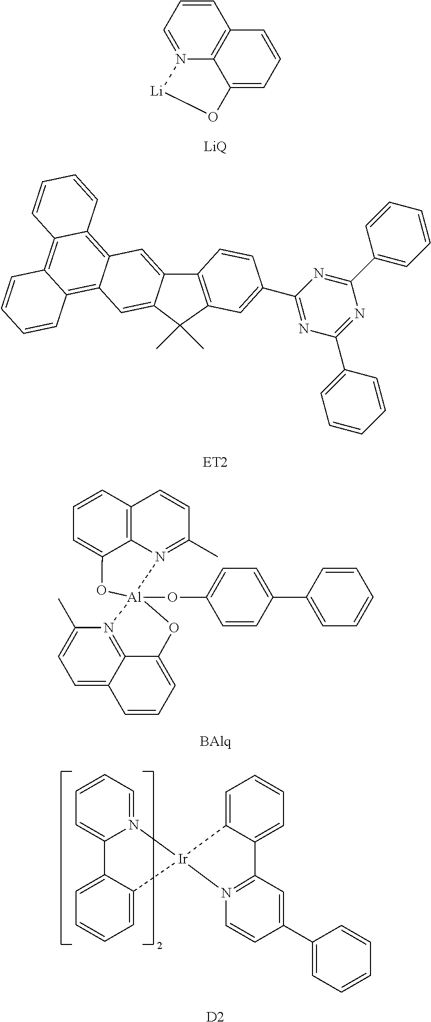

A typical organic EL device consists of low work function metals, such as Al, Mg, Ca, Li and K, as the cathode by thermal evaporation, and the low work function metals can help electrons injecting the electron transporting layer from cathode. In addition, for reducing the electron injection barrier and improving the organic EL device performance, a thin-film electron injecting layer is introduced between the cathode and the electron transporting layer. Conventional materials of electron injecting layer are metal halide or metal oxide with low work function, such as: LiF, LiQ, MgO, or Li₂O. On the other hand, after the organic EL device fabrication, EL spectra and CIE coordination are measured by using a PR650 spectra scan spectrometer. Furthermore, the current/voltage, luminescence/voltage and yield/voltage characteristics are taken with a Keithley 2400 programmable voltage-current source. The above-mentioned apparatuses are operated at room temperature (about 25° C.) and under atmospheric pressure.

### Example 75

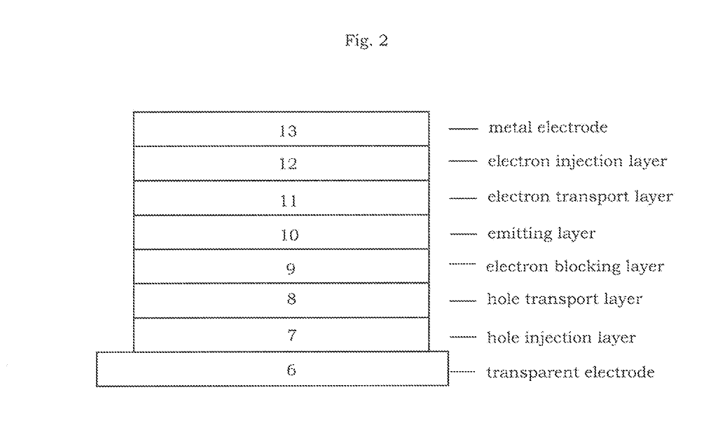

Using a procedure analogous to the above mentioned general method, fluorescent blue-emitting organic EL device having the following device structure was produced (See FIG. 2): ITO/HAT-CN (20 nm)/NPB (130 nm)/electron blocking material (EBM)(5 nm)/PT-312 doped 5% dopant (30 nm)/ET1 co-deposit 5% Li (35 nm)/Al (160 nm). The I-V-B (at 1000 nits) and half-life time of fluorescent blue-emitting organic EL device testing report as Table 1, The half-life time is defined that the initial luminance of 1000 cd/m² has dropped to half.

TABLE 1

| | | Voltage | Efficiency | CIE | Half-life time |
|---|---|---|---|---|---|
| EBM | Dopant | (V) | (cd/A) | (y) | (hour) |
| — | D1 | 5.1 | 4.6 | 0.178 | 240 |
| — | Ex. 74 | 4.8 | 4.1 | 0.181 | 210 |
| Ex. 4 | D1 | 4.8 | 4.6 | 0.180 | 300 |
| Ex. 7 | D1 | 5.0 | 4.3 | 0.181 | 380 |
| Ex. 9 | D1 | 4.4 | 4.2 | 0.180 | 360 |
| Ex. 15 | D1 | 4.5 | 5.0 | 0.182 | 410 |

### Example 76

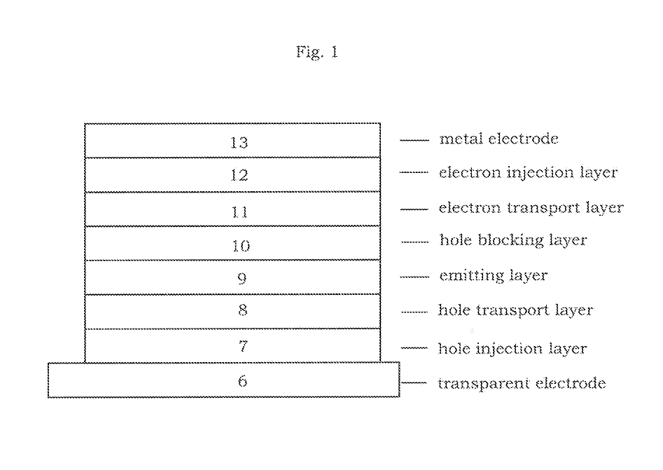

Using a procedure analogous to the above mentioned general method, fluorescent blue-emitting organic EL device having the following device structure was produced (See FIG. 1): ITO/HAT-CN (20 nm)/NPB (130 nm)/PT-312 doped 5% D1 (30 nm)/BAlq (5 nm)/ETM co-deposit 5% Li or ETM co-deposit 50% LiQ/LiQ (1 nm)/Al (160 nm). The I-V-B (at 1000 nits) and half-life time of fluorescent blue-emitting organic EL device testing report as Table 1, The half-life time is defined that the initial luminance of 1000 cd/m² has dropped to half.

TABLE 2

| | 5% Li or | Voltage | Efficiency | CIE | Half-life time |
|---|---|---|---|---|---|
| ETM | 50% LiQ | (V) | (cd/A) | (y) | (hour) |
| ET1 | 5% Li | 5.0 | 4.0 | 0.180 | 250 |
| ET2 | 50% LiQ | 4.6 | 4.3 | 0.181 | 290 |
| Ex. 32 | 5% Li | 4.8 | 3.6 | 0.180 | 200 |
| Ex. 33 | 50% LiQ | 5.0 | 4.6 | 0.181 | 360 |
| Ex. 36 | 5% Li | 4.5 | 4.0 | 0.180 | 160 |
| Ex. 34 | 50% LiQ | 4.5 | 4.5 | 0.180 | 320 |

### Example 77

Using a procedure analogous to the above mentioned general method, fluorescent blue-emitting organic EL device having the following device structure was produced (See FIG. 1): ITO/HAT-CN (20 nm)/NPB (130 nm)/blue host doped 5% D1 (30 nm)/HBM (hole blocking material (5 nm)/ET1 co-deposit 5% Li/Al (160 nm). The I-V-B (at 1000 nits) and half-life time of fluorescent blue-emitting organic EL device testing report as Table 1, The half-life time is defined that the initial luminance of 1000 cd/m² has dropped to half.

TABLE 3

| | | Voltage | Efficiency | CIE | Half-life time |
|---|---|---|---|---|---|
| Blue host | HBM | (V) | (cd/A) | (y) | (hour) |
| PT-312 | BAlq | 6.2 | 4.2 | 0.178 | 240 |
| PT-312 | Ex. 67 | 5.2 | 4.8 | 0.177 | 210 |
| PT-312 | Ex. 70 | 4.8 | 4.6 | 0.176 | 200 |
| Ex. 25 | BAlq | 4.5 | 4.6 | 0.177 | 160 |
| Ex. 25 | EX67 | 4.5 | 5.2 | 0.177 | 180 |
| Ex. 28 | EX67 | 5.5 | 3.4 | 0.178 | 160 |
| Ex. 29 | EX67 | 5.0 | 3.0 | 0.178 | 120 |
| Ex. 30 | EX67 | 4.5 | 6.0 | 0.177 | 180 |

### Example 78

Using a procedure analogous to the above mentioned general method, phosphorescent emitting organic EL device having the following device structures are produced (See FIG. 1.): ITO/HAT-CN (20 nm)/NPB (130 nm)/phosphorescent host (PHhost)+15% D2 (30 nm)/HBM (15 nm)/ET2 co-deposit LiQ (ET2: LiQ, ratio=1:1)(40 nm)/LiQ (1 nm)/Al (160 nm). The I-V-B (at 1000 nits) and half-life time of phosphorescent emitting organic EL device testing report as Table 2. The half-life time is defined that the initial luminance of 3000 cd/m² has dropped to half.

TABLE 4

| PHhost | | | | | |
|---|---|---|---|---|---|
| (H1 + H2) | | Voltage | Efficiency | CIE | Half-life time |
| H1:H2 = 1:1 | HBM | (V) | (cd/A) | (x, y) | (hour) |
| BAlq | BAlq | 6.8 | 35 | 0.46, 0.57 | 320 |
| Ex. 54 + | BAlq | 5.3 | 45 | 0.43, 0.57 | 450 |
| Ex. 66 | | | | | |
| Ex. 54 + | Ex. 67 | 3.8 | 50 | 0.43, 0.57 | 550 |
| Ex. 66 | | | | | |
| Ex. 41 + | Ex. 67 | 4.0 | 48 | 0.43, 0.56 | 600 |
| Ex. 66 | | | | | |
| Ex. 41 + | Ex. 67 | 4.0 | 45 | 0.43, 0.57 | 650 |
| Ex. 73 | | | | | |
| Ex. 26 + | Ex. 67 | 3.7 | 42 | 0.43, 0.56 | 780 |
| Ex. 66 | | | | | |
| Ex. 26 + | Ex. 67 | 3.8 | 51 | 0.43, 0.56 | 600 |
| Ex. 73 | | | | | |

In the above preferred embodiments for organic EL device test report (see Table 1 to Table 4), we shown that the phenanthro[9,10-b]tetra phenylene derivative with a general formula(I) in the present invention display good performance than the prior art of OLED materials.

To sum up, the present invention discloses a phenanthro[9, 10-b]tetraphenylene derivative which can be used for organic EL device is disclosed. More specifically, an organic EL device employing the phenanthro[9,10-b]tetraphenylene derivative as emitting host or dopant, hole blocking layer(HBL), electron blocking layer(EBL), electron transport layer(ETL) and hole transport layer(HTL). The mentioned phenanthro[9,10-b]tetraphenylene derivative are represented by the following formula(I):

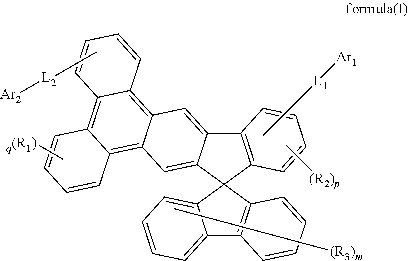

Wherein L₁, L₂ represent a single bond, a substituted or unsubstituted arylene group having 6 to 30 ring carbon atoms, or a substituted or unsubstituted heterarylene group having 3 to 30 ring carbon atoms. m represent an integer of 0 to 8. p represent an integer of 0 to 3, q represent an integer of 0 to 9. R₁ to R₃ independently selected from the group consisting of a hydrogen atom, a halide, alkyl group having 1 to 20 carbon atoms, a substituted or unsubstituted aryl group having 6 to 30 carbon atoms, a substituted or unsubstituted aralkyl group having 6 to 30 carbon atoms, a substituted or unsubstituted heteroaryl group having 3 to 30 carbon atoms. Ar₁ and Ar₂ independently represent a substituted or unsubstituted arylamine, a substituted or unsubstituted heteroarylamine, a substituted or unsubstituted aryl group having 6 to 50 carbon atoms, a substituted or unsubstituted heteroaryl group having 3 to 50 carbon atoms. wherein at least one of Ar₁ and Ar₂ represent a substituted or unsubstituted diphenylamine group, a substituted or unsubstituted N-phenylnaphthalene-2-amine group, a substituted or unsubstituted dibiphenyl-4-ylamine group, a substituted or unsubstituted N-phenyldibenzo[b,d]furan-4-amine group, a substituted or unsubstituted phenyl group, a substituted or unsubstituted naphthyl group, a substituted or unsubstituted anthracenyl group, a substituted or unsubstituted phenanthrenyl group, a substituted or unsubstituted pyrenyl group, a substituted or unsubstituted chrysenyl group, a substituted or unsubstituted triphenylenyl group, a substituted or unsubstituted perylenyl group, a substituted or unsubstituted carbazolyl group, a substituted or unsubstituted biscarbazolyl group, a substituted or unsubstituted dibenzofuranyl group, a substituted or unsubstituted dibenzothiophenyl group, a substituted or unsubstituted triazinyl group, a substituted or unsubstituted diazinyl group, a substituted or unsubstituted phenanthroline group, a substituted or unsubstituted dihydroacridine group, a substituted or unsubstituted phenothiazine group, a substituted or unsubstituted phenoxazine group, a substituted or unsubstituted dihydrophenazine group and the substituent for Ar₁, Ar₂ each have the same definition as R₁.

1. A phenanthro[9,10-b]tetraphenylene derivative with a general formula(I) as following: 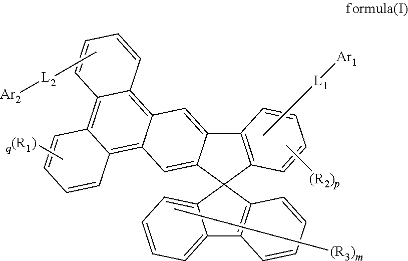
Wherein L₁, L₂ represent a single bond, a substituted or unsubstituted arylene group having 6 to 30 ring carbon atoms, or a substituted or unsubstituted heterarylene group having 3 to 30 ring carbon atoms. m represent an integer of 0 to 8. p represent an integer of 0 to 3, q represent an integer of 0 to 9. R₁ to R₃ independently selected from the group consisting of a hydrogen atom, a halide, alkyl group having 1 to 20 carbon atoms, a substituted or unsubstituted aryl group having 6 to 30 carbon atoms, a substituted or unsubstituted aralkyl group having 6 to 30 carbon atoms, a substituted or unsubstituted heteroaryl group having 3 to 30 carbon atoms. Ar₁ and Ar₂ independently represent a substituted or unsubstituted arylamine, a substituted or unsubstituted heteroarylamine, a substituted or unsubstituted aryl group having 6 to 50 carbon atoms, a substituted or unsubstituted heteroaryl group having 3 to 50 carbon atoms. wherein at least one of Ar₁ and Ar₂ represent a substituted or unsubstituted diphenylamine group, a substituted or unsubstituted N-phenylnaphthalene-2-amine group, a substituted or unsubstituted dibiphenyl-4-ylamine group, a substituted or unsubstituted N-phenyldibenzo[b,d]furan-4-amine group, a substituted or unsubstituted phenyl group, a substituted or unsubstituted naphthyl group, a substituted or unsubstituted anthracenyl group, a substituted or unsubstituted phenanthrenyl group, a substituted or unsubstituted pyrenyl group, a substituted or unsubstituted chrysenyl group, a substituted or unsubstituted triphenylenyl group, a substituted or unsubstituted perylenyl group, a substituted or unsubstituted carbazolyl group, a substituted or unsubstituted biscarbazolyl group, a substituted or unsubstituted dibenzofuranyl group, a substituted or unsubstituted dibenzothiophenyl group, a substituted or unsubstituted triazinyl group, a substituted or unsubstituted diazinyl group, a substituted or unsubstituted phenanthroline group, a substituted or unsubstituted dihydroacridine group, a substituted or unsubstituted phenothiazine group, a substituted or unsubstituted phenoxazine group, a substituted or unsubstituted dihydrophenazine group and the substituent for Ar₁, Ar₂ each have the same definition as R₁.

2. According to claim 1, Ar₁ and Ar₂ are consisting of group represent as following:

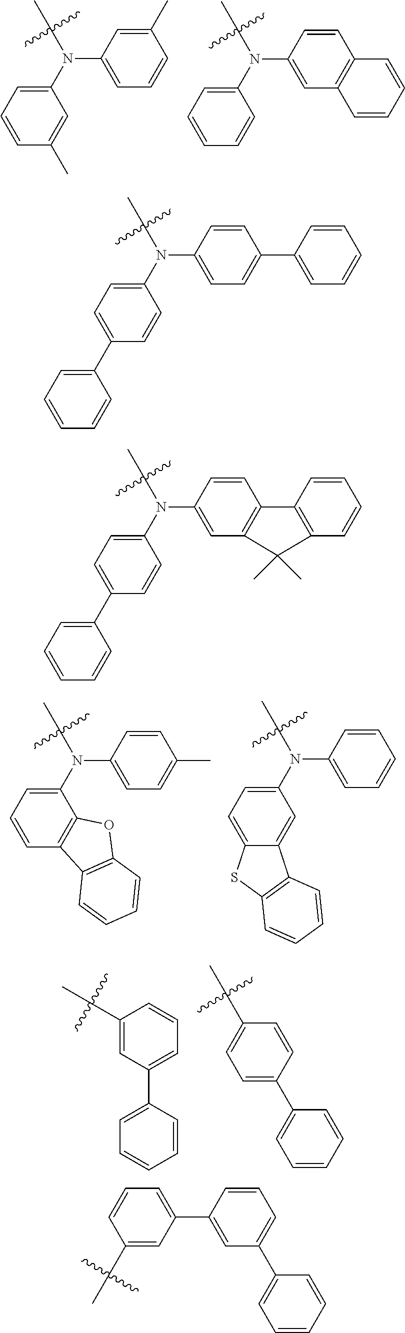

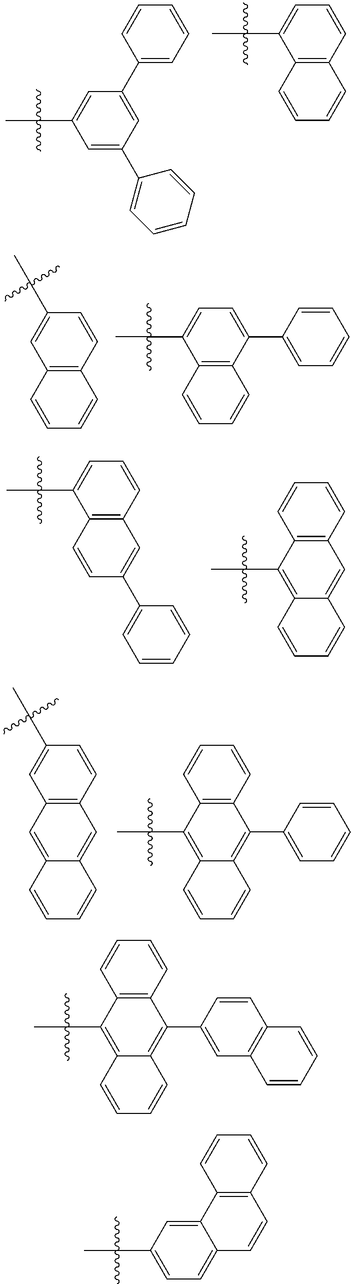

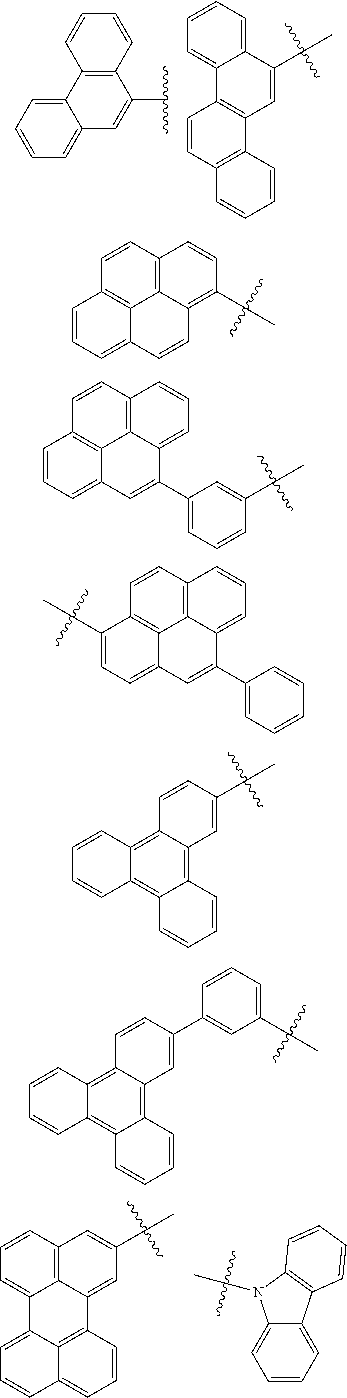

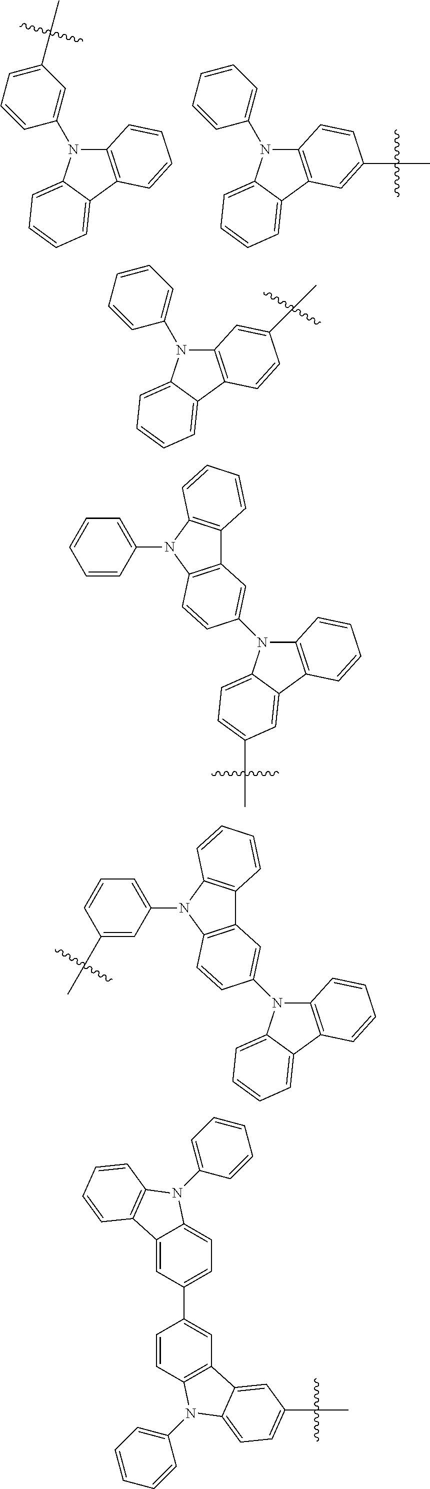

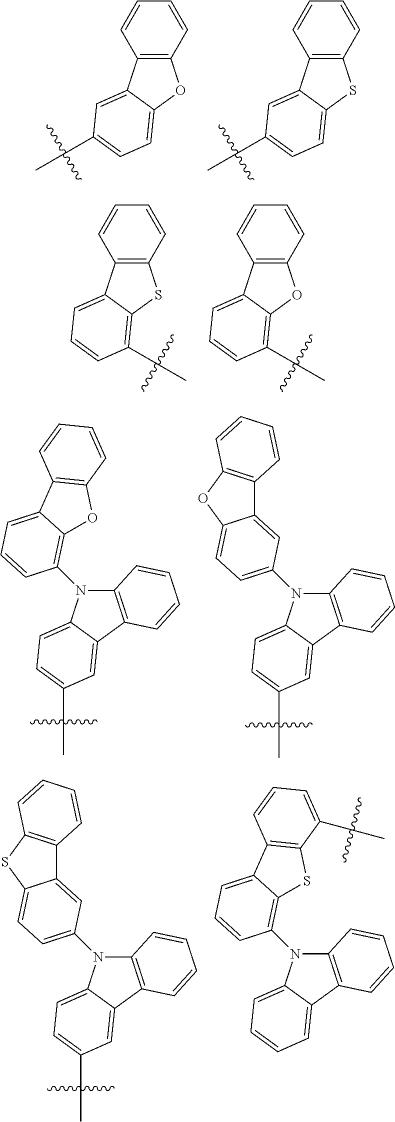

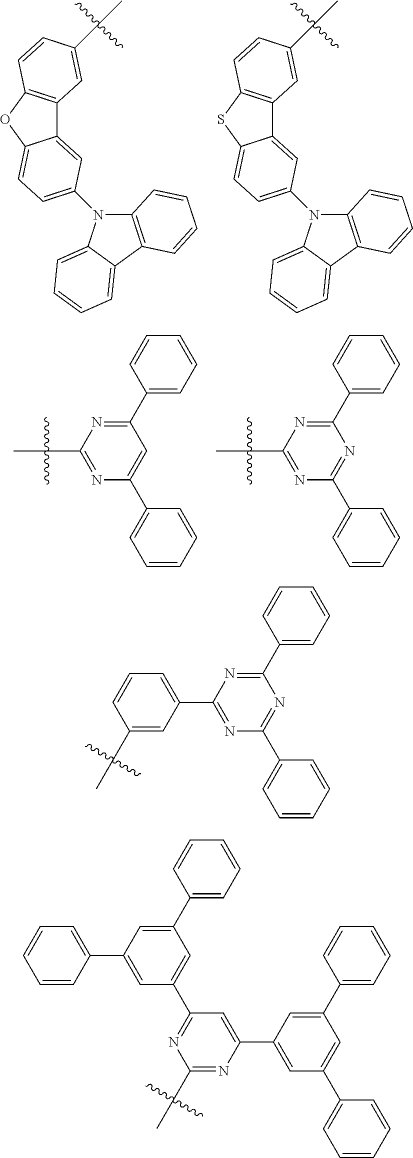

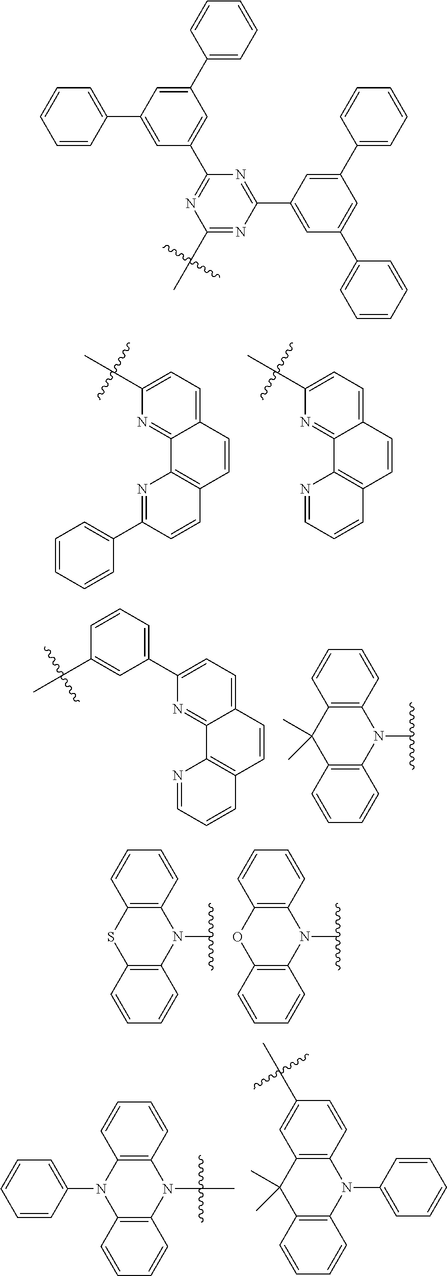

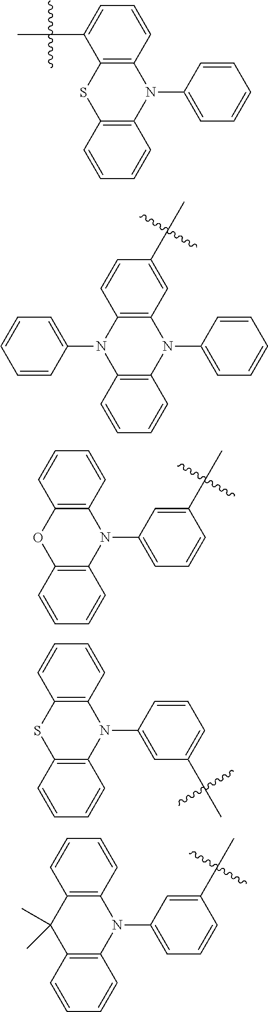

3. According to claim 1, when L₁, L₂ are not represented single bond, the arylene group and heterarylene group for L₁ and L₂ are consisting of group represent as:

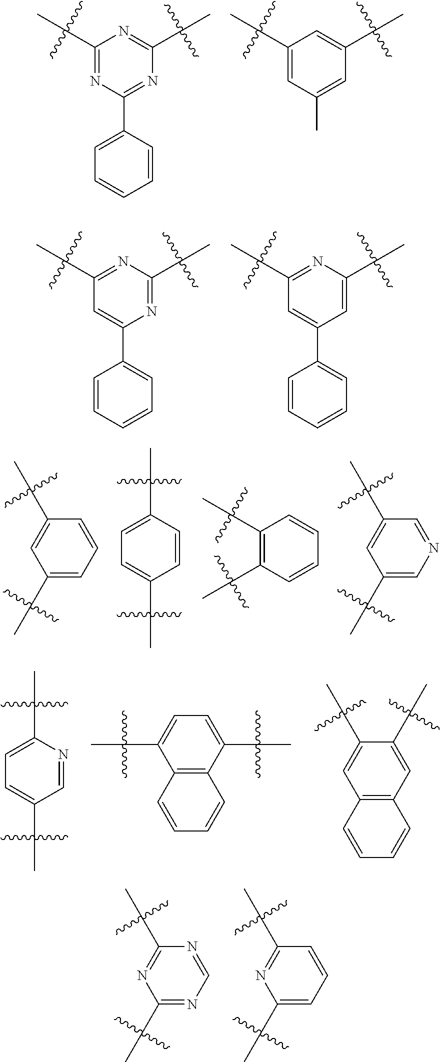

4. According to claim 1, when L₁, L₂ are simultaneously represented single bond and Ar₁, Ar₂ are different, some preferable group of Ar₁ and Ar₂ are consisting of group represent as:

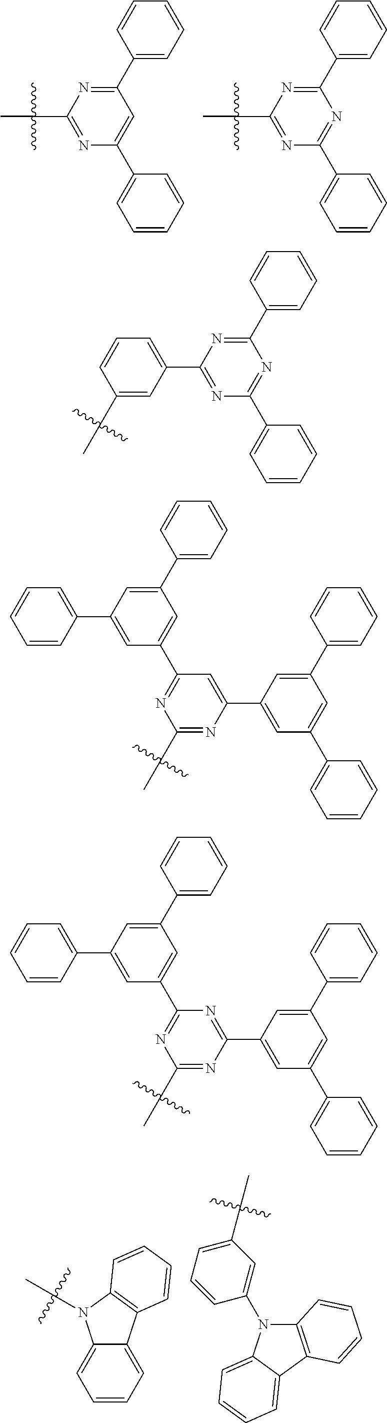

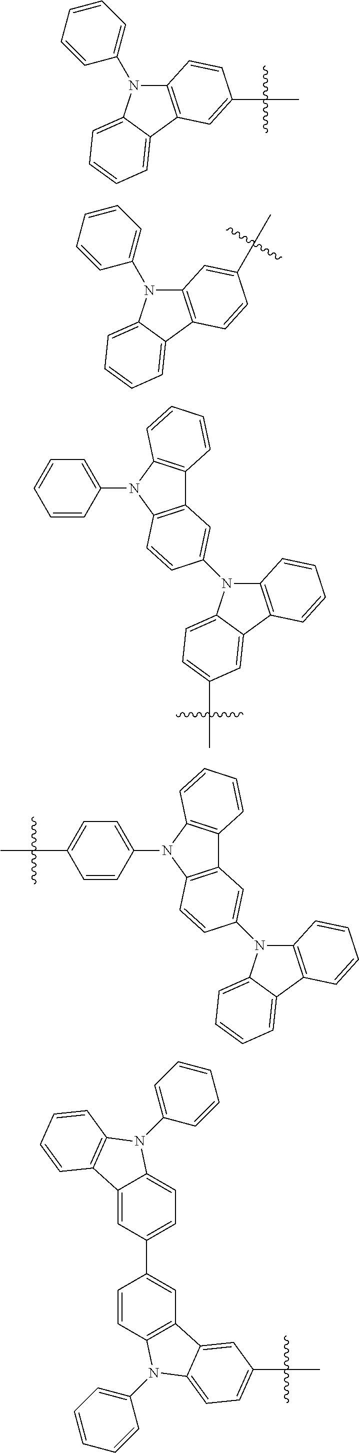

5. A organic electroluminescent device comprising a pair of electrodes consisting of a cathode and an anode and between the pairs of electrodes comprising at least a layer of the phenanthro[9,10-b]tetraphenylene derivative with a general formula(I) according to claim 1.

6. The organic electroluminescent device according to claim 5, wherein the emitting layer comprising the phenanthro[9,10-b]tetraphenylene derivative with a general formula(I).

7. The organic electroluminescent device according to claim 6, wherein the emitting layer comprising the phenanthro[9,10-b]tetraphenylene derivative with a general formula(I) is a fluorescent host material, a phosphorescent host material or a thermally activated delayed fluorescence host material.

8. The organic electroluminescent device according to claim 6, wherein the emitting layer comprising fluorescent dopant, phosphorescent dopant or thermally activated delayed fluorescence dopant.

9. The organic electroluminescent device according to claim 8, wherein the phosphorescent dopant are iridium (Ir) complexes.

10. The organic electroluminescent device according to claim 5, wherein the electron transport layer comprising the phenanthro[9,10-b]tetraphenylene derivative with a general formula(I).

11. The organic electroluminescent device according to claim 10, wherein the electron transport layer comprising lithium, calcium, 8-hydroxyquinolinolato-lithium.

12. The organic electroluminescent device according to claim 5, wherein the hole blocking layer or electron blocking layer comprising the phenanthro[9, 10-b]tetraphenylene derivative with a general formula(I).

13. According to claim 1, the phenanthro[9,10-b]tetraphenylene derivative with a general formula(I) are 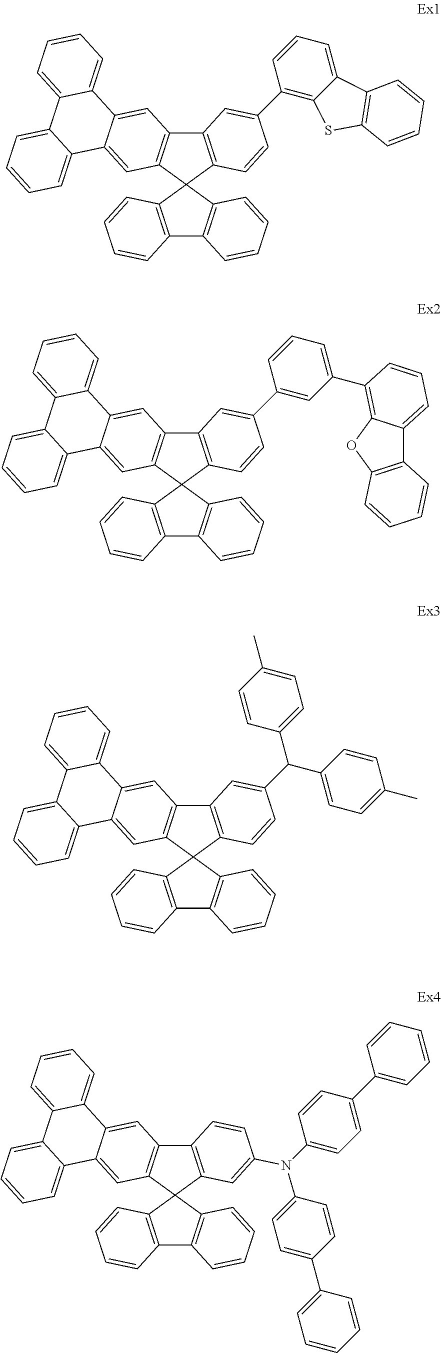 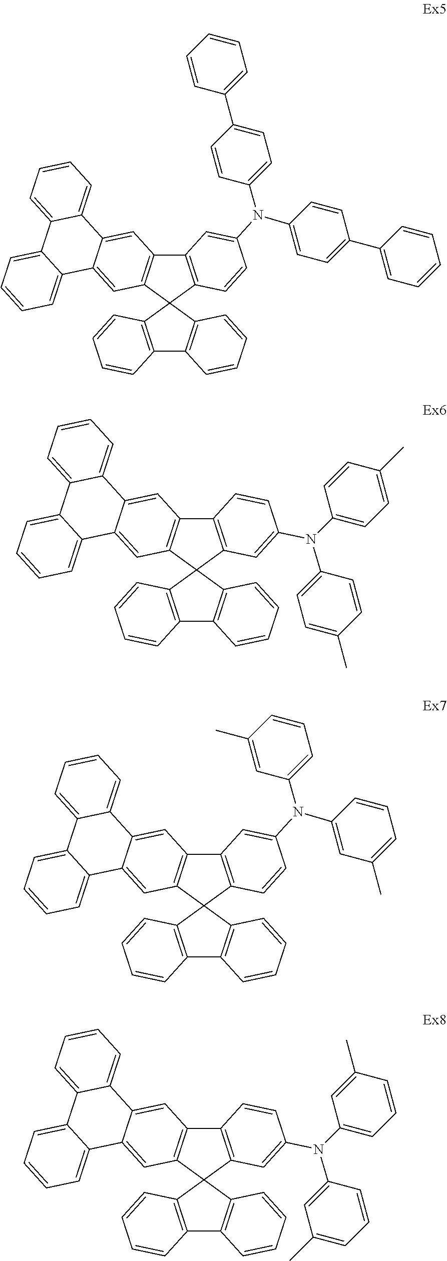 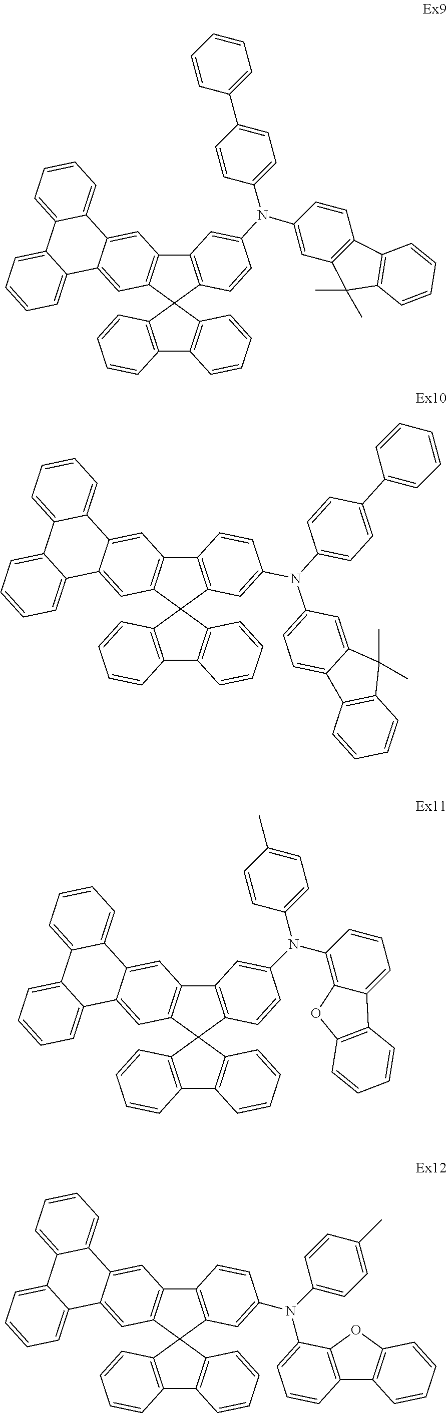 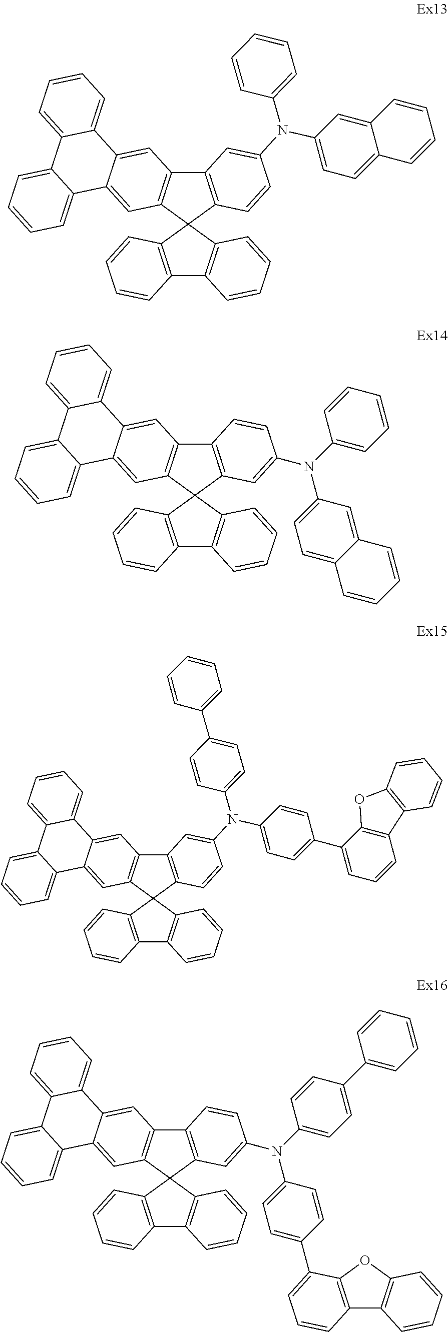 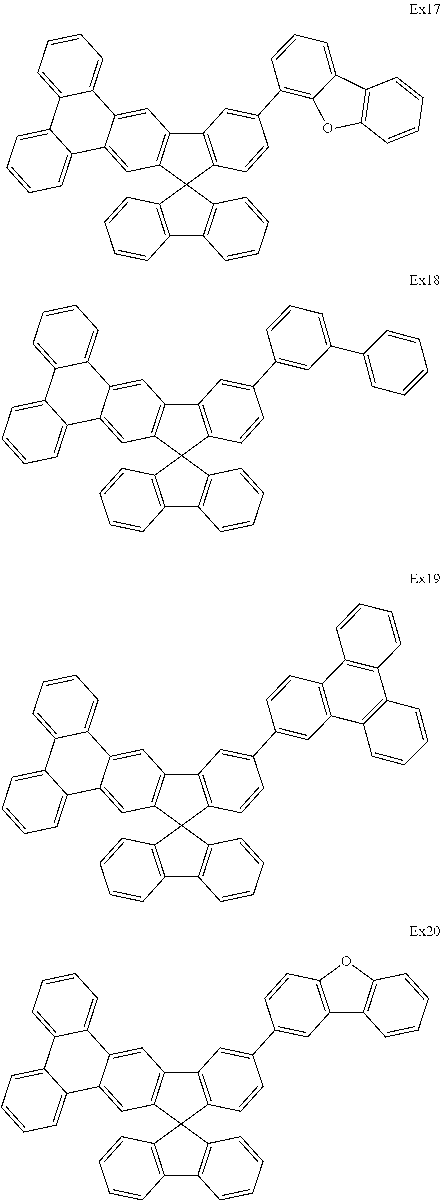 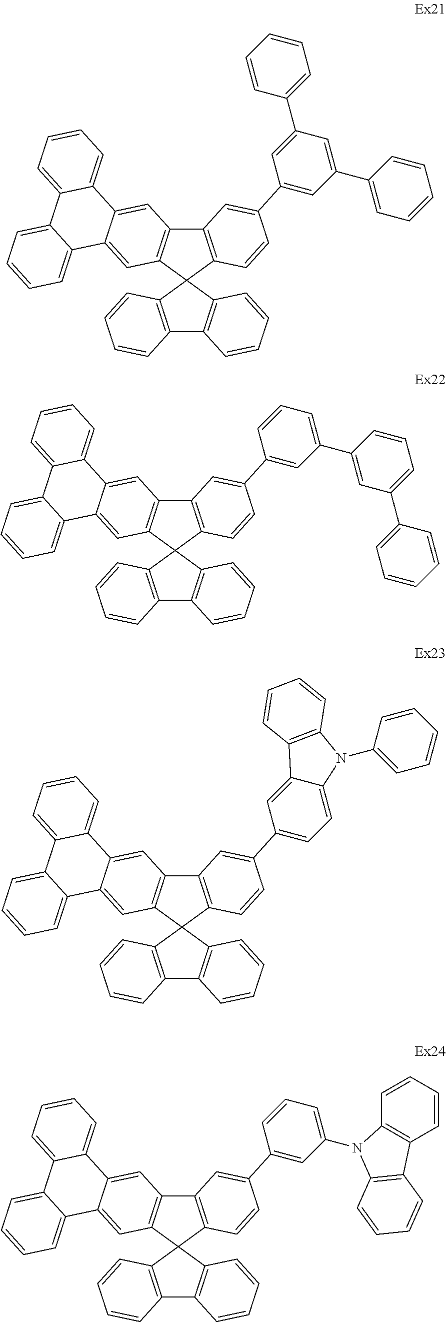 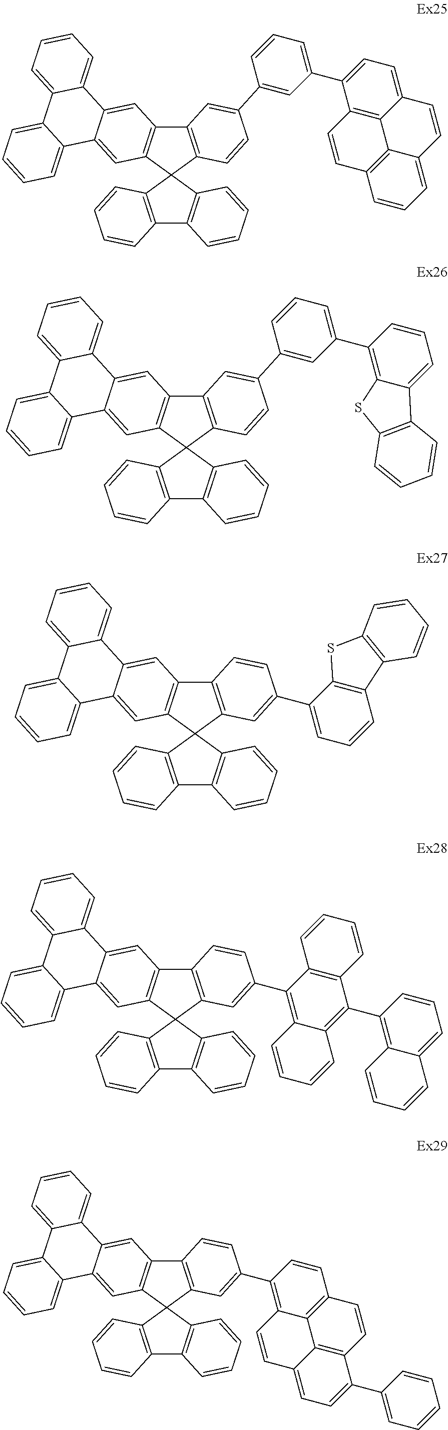 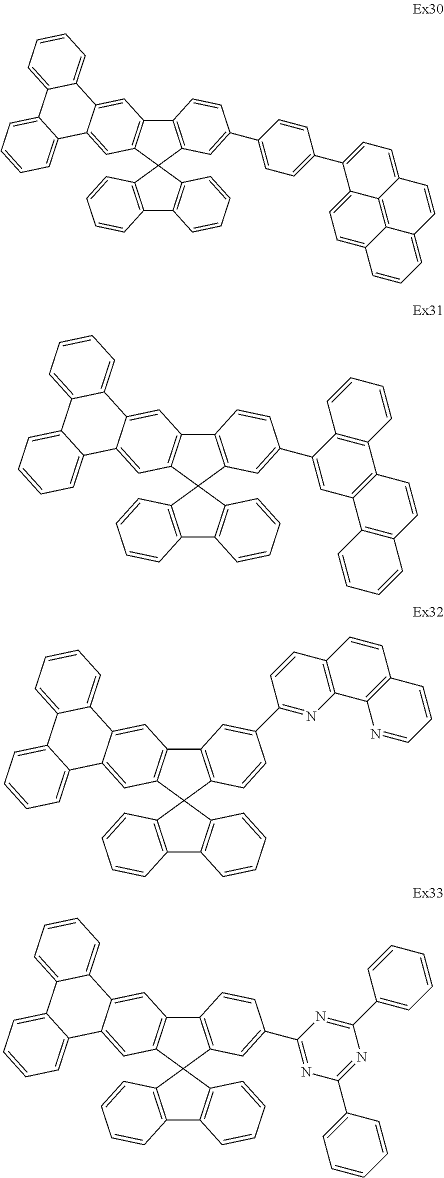 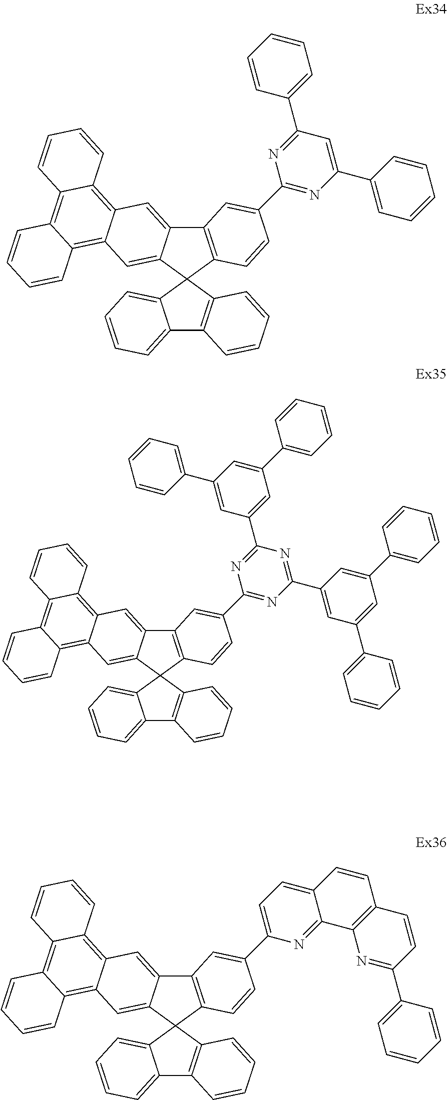 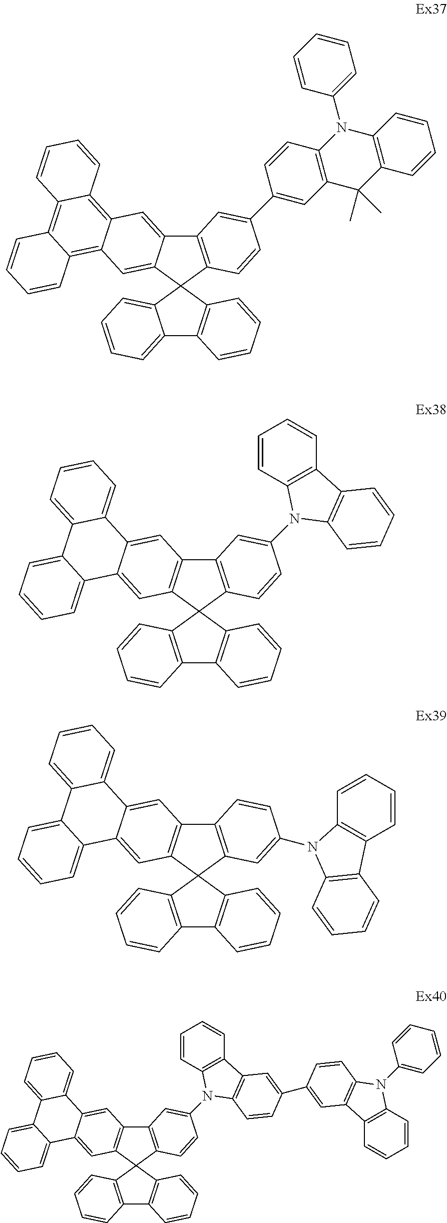 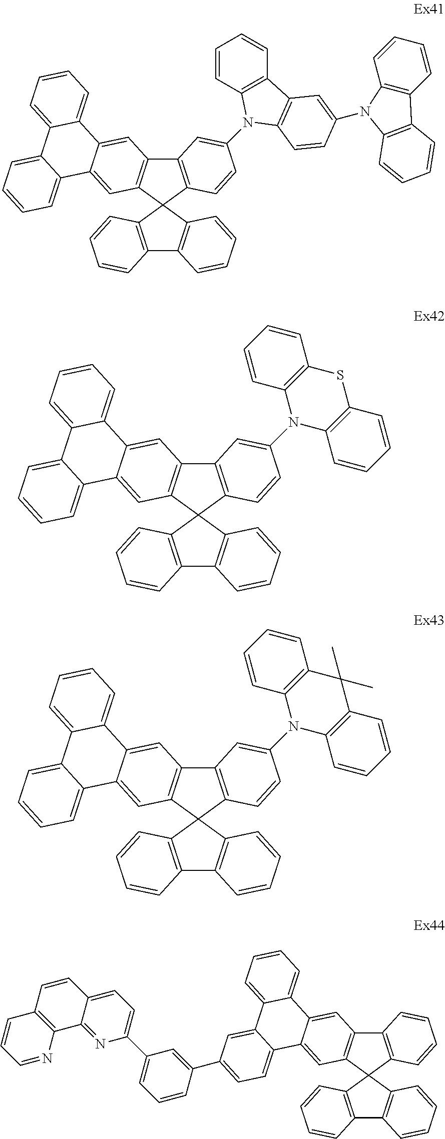 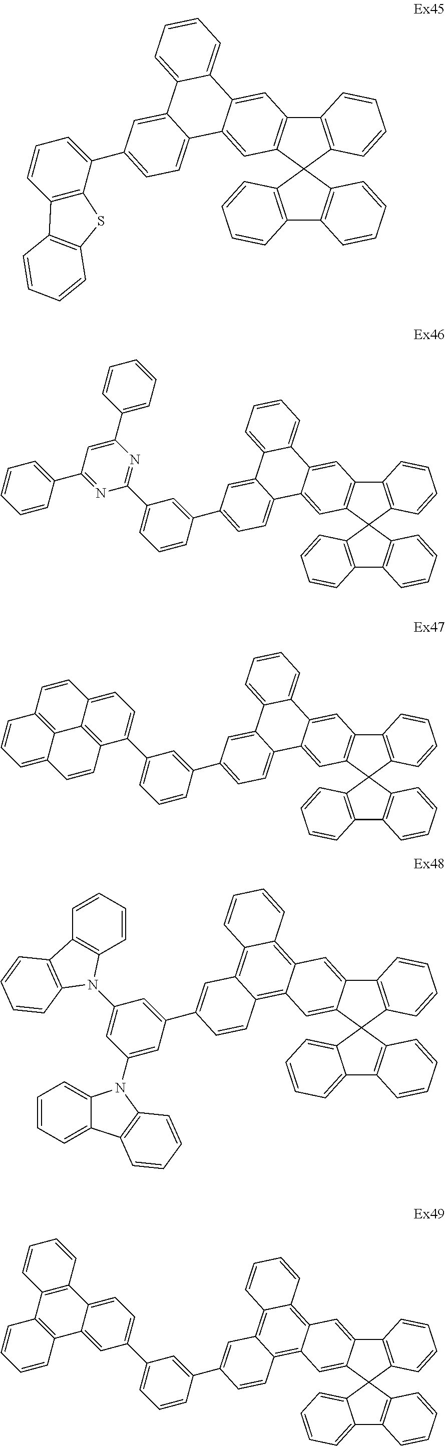 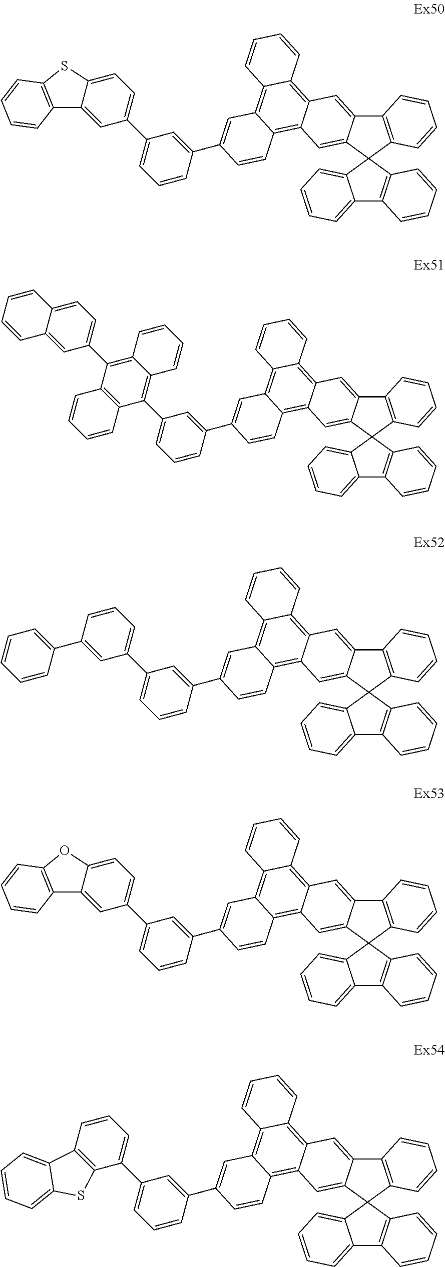 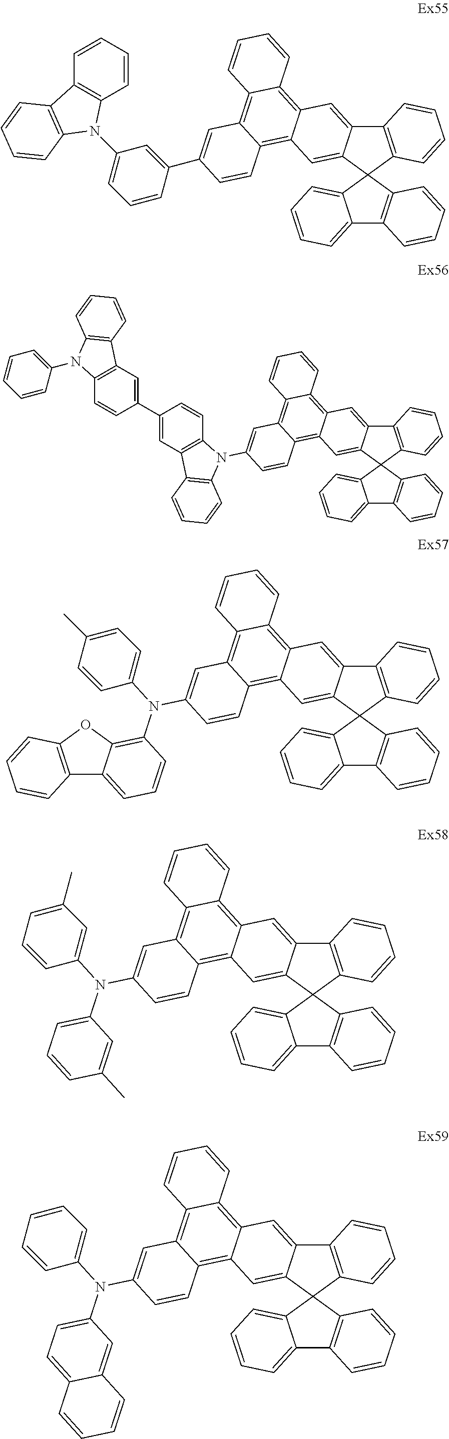 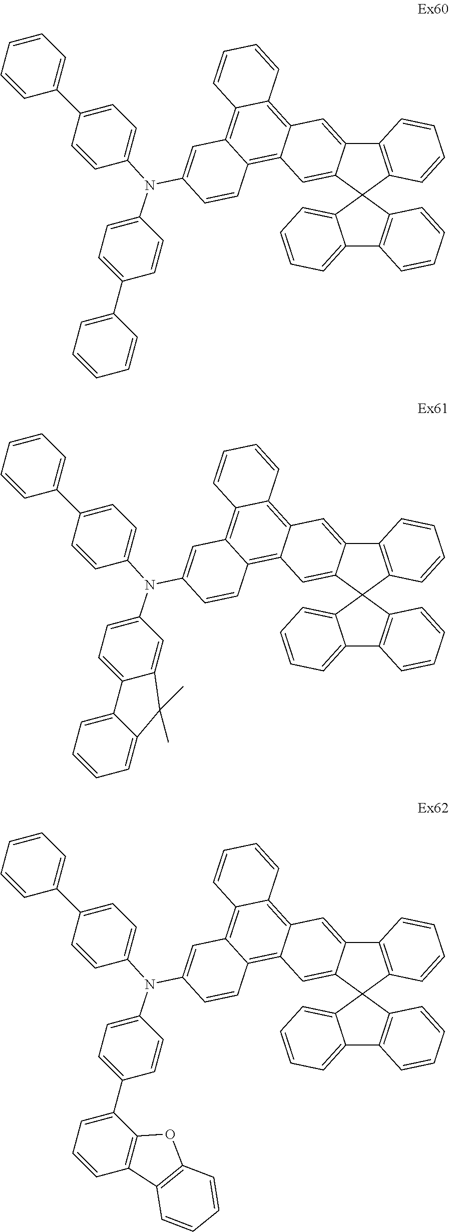 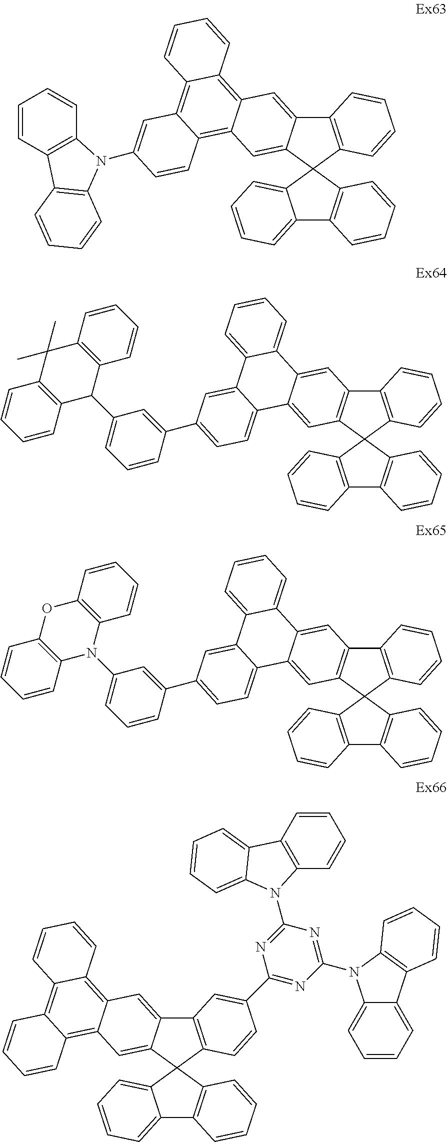 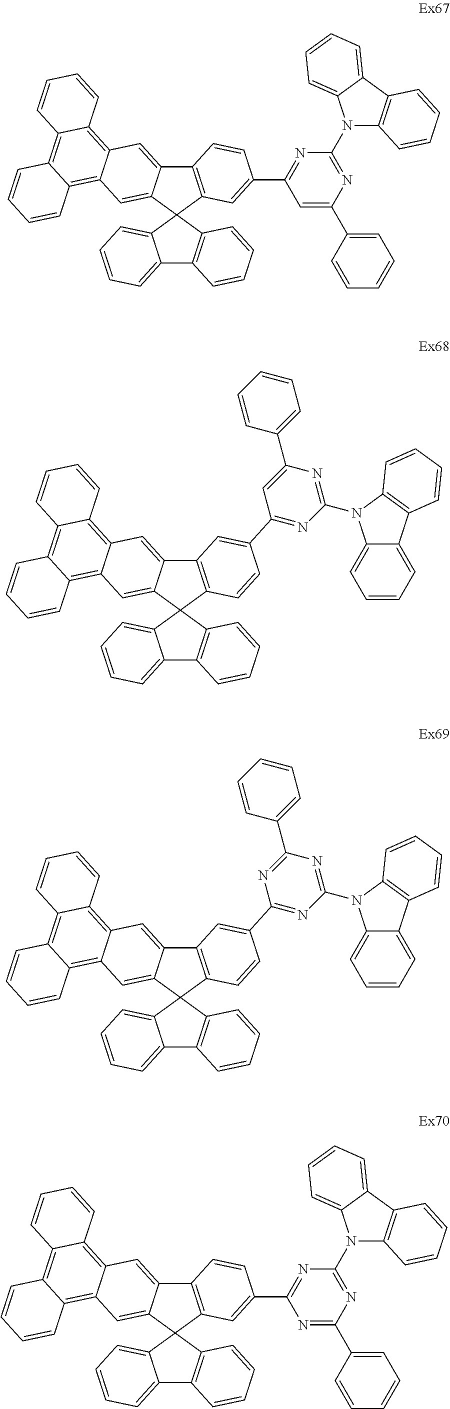 [chemical structure]
